SEQUENCE LISTING (288 sequences)

SEQ ID NO: 1 - DNA, 41 bp, synthetic construct
  source 1..41: PCR_primers = fwd_seq: cggcccttgtgtaaacctgtc, rev_seq: gcacggttatacaatgtcct; mol_type = other DNA; note = ORF K1
cggcccttgtgtaaacctgtcgcacggttatacaatgtcct

SEQ ID NO: 2 - DNA, 40 bp, synthetic construct
  source 1..40: PCR_primers = fwd_seq: aggacattgtataaccgtgc, rev_seq: gttgtcttgtattggtcggt; mol_type = other DNA; note = ORF 2
aggacattgtataaccgtgcgttgtcttgtattggtcggt

SEQ ID NO: 3 - DNA, 41 bp, Synthetic construct
  source 1..41: PCR_primers = fwd_seq: tatagtgcgcggtgtggcag, rev_seq: gagtagtgtgccgtgaaggct; mol_type = other DNA; note = ORF 4
tatagtgcgcggtgtggcaggagtagtgtgccgtgaaggct

SEQ ID NO: 4 - DNA, 43 bp, Synthetic construct
  source 1..43: PCR_primers = fwd_seq: acagtcggtagtggaggagc, rev_seq: gctctgaaacttccctgtagtga; mol_type = other DNA; note = ORF 6
acagtcggtagtggaggagcgctctgaaacttccctgtagtga

SEQ ID NO: 5 - DNA, 44 bp, synthetic construct
  source 1..44: PCR_primers = fwd_seq: gaggaaccgaaacccgcagg, rev_seq: ccacactcttaggaccagatgctt; mol_type = other DNA; note = ORF 7
gaggaaccgaaacccgcaggccacactcttaggaccagatgctt

SEQ ID NO: 6 - DNA, 40 bp, synthetic construct
  source 1..40: PCR_primers = fwd_seq: caatcgctagacatcagtcc, rev_seq: cgttatctcccagtcaccta; mol_type = other DNA; note = ORF 8
caatcgctagacatcagtcccgttatctcccagtcaccta

SEQ ID NO: 7 - DNA, 41 bp, synthetic construct
  source 1..41: PCR_primers = fwd_seq: agaacaacacgtcggcaacc, rev_seq: ggattcttagccgcgtgtagt; mol_type = other DNA; note = ORF 9
agaacaacacgtcggcaaccggattcttagccgcgtgtagt

SEQ ID NO: 8 - DNA, 48 bp, synthetic construct
  source 1..48: PCR_primers = fwd_seq: ccacactcttaggaccagatgctt, rev_seq:ccacactcttaggaccagatgctt; mol_type = other DNA; note = ORF 10.1
ccacactcttaggaccagatgcttccacactcttaggaccagatgctt

SEQ ID NO: 9 - DNA, 45 bp, synthetic construct
  source 1..45: PCR_primers = fwd_seq: gccaggcaccatacagcttc, rev_seq: gattagagatgacgatgtggctcgg; mol_type = other DNA; note = ORF 10
gccaggcaccatacagcttcgattagagatgacgatgtggctcgg

SEQ ID NO: 10 - DNA, 43 bp, synthetic construct
  source 1..43: PCR_primers = fwd_seq: cagcttctacgactgcaagg, rev_seq: gtgtgtgttatgtattcgggtgg; mol_type = other DNA; note = ORF 11AA
cagcttctacgactgcaagggtgtgtgttatgtattcgggtgg

SEQ ID NO: 11 - DNA, 41 bp, synthetic construct
  source 1..41: PCR_primers = fwd_seq: tgttcccatacgcgcctgtc, rev_seq: ctggcgagcaagagagggttt; mol_type = other DNA; note = ORF 11
tgttcccatacgcgcctgtcctggcgagcaagagagggttt

SEQ ID NO: 12 - DNA, 41 bp, Synthetic construct
  source 1..41: PCR_primers = fwd_seq: cgcgttccagataccagcag, rev_seq: gctggaccctcctctctagtt; mol_type = other DNA; note = ORF K2
cgcgttccagataccagcaggctggaccctcctctctagtt

SEQ ID NO: 13 - DNA, 44 bp, synthetic construct
  source 1..44: PCR_primers = fwd_seq: tggtatcaaccgcaactacacag, rev_seq: gtgagtggctgtagcattacc; mol_type = other DNA; note = ORF 2
tggtatcaaccgcaactacacaggtgagtggctgtagcattacc

SEQ ID NO: 14 - DNA, 44 bp, synthetic construct
  source 1..44: PCR_primers = fwd_seq: gattcatgtagacaacccgctc, rev_seq: cgtgggaactgtgagtaatgtg; mol_type = other DNA; note = ORF K3
gattcatgtagacaacccgctccgtgggaactgtgagtaatgtg

SEQ ID NO: 15 - DNA, 40 bp, synthetic construct
  source 1..40: PCR_primers = fwd_seq: cgctgttctcctgtaattgg, rev_seq: ggtgttggccttcgtaataa; mol_type = other DNA; note = ORF 70
cgctgttctcctgtaattggggtgttggccttcgtaataa

SEQ ID NO: 16 - DNA, 40 bp, synthetic construct
  source 1..40: PCR_primers = fwd_seq: acgacggttacaggtccctc, rev_seq: catttggcaacgctggtctt; mol_type = other DNA; note = ORF K4
acgacggttacaggtccctccatttggcaacgctggtctt

SEQ ID NO: 17 - DNA, 42 bp, synthetic construct
  source 1..42: PCR_primers = fwd_seq: ctggccgcaatagctcaatc , rev_seq: ccgctaacagcaccaaatccac; mol_type = other DNA; note = ORF K4.1
ctggccgcaatagctcaatcccgctaacagcaccaaatccac

SEQ ID NO: 18 - DNA, 40 bp, synthetic construct
  source 1..40: PCR_primers = fwd_seq: aggacagatttgggcacagg, rev_seq: cagggtgggttgtgacagtt; mol_type = other DNA; note = ORF K4.2
aggacagatttgggcacaggcagggtgggttgtgacagtt

SEQ ID NO: 19 - DNA, 43 bp, synthetic construct
  source 1..43: PCR_primers = fwd_seq: catttcttcctcgacaggtcttc, rev_seq: gcatgtaagctggcggttag; mol_type = other DNA; note = ORF K5
catttcttcctcgacaggtcttcgcatgtaagctggcggttag

SEQ ID NO: 20 - DNA, 45 bp, synthetic construct
  source 1..45: PCR_primers = fwd_seq: ttatggattattaagggtcagcttg, rev_seq: cgacgcaatcaacccacaat; mol_type = other DNA; note = ORF K6
ttatggattattaagggtcagcttgcgacgcaatcaacccacaat

SEQ ID NO: 21 - DNA, 42 bp, synthetic construct
  source 1..42: PCR_primers = fwd_seq: ggaaccagcttggtgatgtg, rev_seq: caagcagtagcgaacagttacg; mol_type = other DNA; note = ORF K7
ggaaccagcttggtgatgtgcaagcagtagcgaacagttacg

SEQ ID NO: 22 - DNA, 45 bp, synthetic construct
  source 1..45: PCR_primers = fwd_seq: ggcaggacacaacatctacaaac, rev_seq: cattggcagtagcctcccttaa; mol_type = other DNA; note = ORF 16
ggcaggacacaacatctacaaaccattggcagtagcctcccttaa

SEQ ID NO: 23 - DNA, 44 bp, synthetic construct
  source 1..44: PCR_primers = fwd_seq: taccgtgggacacttgaaatagac, rev_seq: gctcttgggcgtggaatgta; mol_type = other DNA; note = ORF 17
taccgtgggacacttgaaatagacgctcttgggcgtggaatgta

SEQ ID NO: 24 - DNA, 42 bp, synthetic construct
  source 1..42: PCR_primers = fwd_seq: cattacatcgctcactcggccc, rev_seq: cggtgaagttacggtggctg; mol_type = other DNA; note = ORF 18
cattacatcgctcactcggccccggtgaagttacggtggctg

SEQ ID NO: 25 - DNA, 41 bp, synthetic construct
  source 1..41: PCR_primers = fwd_seq: gatccattggcgcgtagtctc, rev_seq: agctcggacgacgaatcaag; mol_type = other DNA; note = ORF 19
gatccattggcgcgtagtctcagctcggacgacgaatcaag

SEQ ID NO: 26 - DNA, 42 bp, synthetic construct
  source 1..42: PCR_primers = fwd_seq: cggcaattctgtgccctagag, rev_seq: gtgcttgattcgtcgtccgag; mol_type = other DNA; note = ORF 20
cggcaattctgtgccctagaggtgcttgattcgtcgtccgag

SEQ ID NO: 27 - DNA, 40 bp, synthetic construct
  source 1..40: PCR_primers = fwd_seq: cccgtcgtgatcgagctttg, rev_seq: ggagcgggttattgtcgtcg; mol_type = other DNA; note = ORF 21
cccgtcgtgatcgagctttgggagcgggttattgtcgtcg

SEQ ID NO: 28 - DNA, 41 bp, synthetic construct
  source 1..41: PCR_primers = fwd_seq: gtcatcggttcgccgctcta, rev_seq: gaacaacagtggcatcgggac; mol_type = other DNA; note = ORF 22
gtcatcggttcgccgctctagaacaacagtggcatcgggac

SEQ ID NO: 29 - DNA, 43 bp, synthetic construct
  source 1..43: PCR_primers = fwd_seq: ggacactgccttctctggcg, rev_seq: cttctaaggtcagctctgcctgc; mol_type = other DNA; note = ORF 23
ggacactgccttctctggcgcttctaaggtcagctctgcctgc

SEQ ID NO: 30 - DNA, 44 bp, synthetic construct
  source 1..44: PCR_primers = fwd_seq:agtagtctgcgtatcgctctgc, rev_seq: ccttgccgagcaatagctgaaa; mol_type = other DNA; note = ORF 24
agtagtctgcgtatcgctctgcccttgccgagcaatagctgaaa

SEQ ID NO: 31 - DNA, 40 bp, synthetic construct
  source 1..40: PCR_primers = fwd_seq: cgtcgtggtcaacggtacag, rev_seq: caatctccgagcggcagtac; mol_type = other DNA; note = ORF 25
cgtcgtggtcaacggtacagcaatctccgagcggcagtac

SEQ ID NO: 32 - DNA, 43 bp, synthetic construct
  source 1..43: PCR_primers = fwd_seq: tctggacgtagacaacacggatc, rev_seq: gagccgtcatccgtccttgc; mol_type = other DNA; note = ORF 26
tctggacgtagacaacacggatcgagccgtcatccgtccttgc

SEQ ID NO: 33 - DNA, 45 bp, synthetic construct
  source 1..45: PCR_primers = fwd_seq: tttacgctaagagttgggtgcttg, rev_seq: ggacgacttacttgtggcagt; mol_type = other DNA; note = ORF 27
tttacgctaagagttgggtgcttgggacgacttacttgtggcagt

SEQ ID NO: 34 - DNA, 45 bp, synthetic construct
  source 1..45: PCR_primers = fwd_seq: gttcgcctcctctcctttactgtta, rev_seq: ggtggaaatcttcgcggtgg; mol_type = other DNA; note = ORF 28
gttcgcctcctctcctttactgttaggtggaaatcttcgcggtgg

SEQ ID NO: 35 - DNA, 40 bp, synthetic construct
  source 1..40: PCR_primers = fwd_seq:tgggcgttcctgaggttaag, rev_seq: ccaagaacaagagccacgca; mol_type = other DNA; note = ORF 29b
tgggcgttcctgaggttaagccaagaacaagagccacgca

SEQ ID NO: 36 - DNA, 44 bp, synthetic construct
  source 1..44: PCR_primers = fwd_seq:tctgaagcatgaaacccagaatag, rev_seq:cgaccacttgctccctaagg; mol_type = other DNA; note = ORF 30.1
tctgaagcatgaaacccagaatagcgaccacttgctccctaagg

SEQ ID NO: 37 - DNA, 43 bp, synthetic construct
  source 1..43: PCR_primers = fwd_seq: cagtaaaggagaggaggcgaac, rev_seq: ggactcgccacactcttcatt; mol_type = other DNA; note = ORF 30
cagtaaaggagaggaggcgaacggactcgccacactcttcatt

SEQ ID NO: 38 - DNA, 41 bp, synthetic construct
  source 1..41: PCR_primers = fwd_seq: cgtgcaggaaatagccctgg, rev_seq:gtgatgcagcagaaaggtcct; mol_type = other DNA; note = ORF 31
cgtgcaggaaatagccctgggtgatgcagcagaaaggtcct

SEQ ID NO: 39 - DNA, 45 bp, synthetic construct
  source 1..45: PCR_primers = fwd_seq: acggacggtgactggttagag, rev_seq: gagatagaggtgcaggcgttaaag; mol_type = other DNA; note = ORF 32
acggacggtgactggttagaggagatagaggtgcaggcgttaaag

SEQ ID NO: 40 - DNA, 45 bp, synthetic construct; identical to SEQ ID NO: 39
  source 1..45: PCR_primers = fwd_seq: acggacggtgactggttagag, rev_seq: gagatagaggtgcaggcgttaaag; mol_type = other DNA; note = ORF 33

SEQ ID NO: 41 - DNA, 41 bp, synthetic construct
  source 1..41: PCR_primers = fwd_seq: cgttgtcctcggacggtctg, rev_seq: ctactggtcacctccgggtca; mol_type = other DNA; note = ORF 29a
cgttgtcctcggacggtctgctactggtcacctccgggtca

SEQ ID NO: 42 - DNA, 42 bp, synthetic construct
  source 1..42: PCR_primers = fwd_seq: gatttgctgacgtgggcgtg, rev_seq: ccaggtgtgttctcgctaaggt; mol_type = other DNA; note = ORF 34.1
gatttgctgacgtgggcgtgccaggtgtgttctcgctaaggt

SEQ ID NO: 43 - DNA, 42 bp, synthetic construct
  source 1..42: PCR_primers = fwd_seq: ttacatttcccacacctgcctc, rev_seq: ctcaaacacggcgctgctac; mol_type = other DNA; note = ORF 34
ttacatttcccacacctgcctcctcaaacacggcgctgctac

SEQ ID NO: 44 - DNA, 42 bp, synthetic construct
  source 1..42: PCR_primers = fwd_seq: agtttcaggacgcacagcatg, rev_seq: gcgctttaagatacgcggtgg; mol_type = other DNA; note = ORF 35
agtttcaggacgcacagcatggcgctttaagatacgcggtgg

SEQ ID NO: 45 - DNA, 40 bp, synthetic construct
  source 1..40: PCR_primers = fwd_seq:gattcccatgcgacaggagc, rev_seq:cagtctcgaaccttggcgtg; mol_type = other DNA; note = ORF 36
gattcccatgcgacaggagccagtctcgaaccttggcgtg

SEQ ID NO: 46 - DNA, 42 bp, synthetic construct
  source 1..42: PCR_primers = fwd_seq: cctgtcaactgtaccatcggtg, rev_seq: ggattgcgattgctcaagca; mol_type = other DNA; note = ORF 37
cctgtcaactgtaccatcggtgggattgcgattgctcaagca

SEQ ID NO: 47 - DNA, 40 bp, synthetic construct
  source 1..40: PCR_primers = fwd_seq: gggatggaggaagagggatg, rev_seq: ggacgtgaacgctgtgaaag; mol_type = other DNA; note = ORF 38
gggatggaggaagagggatgggacgtgaacgctgtgaaag

SEQ ID NO: 48 - DNA, 43 bp, synthetic construct
  source 1..43: PCR_primers = fwd_seq: gcgggagagccaatctgatg, rev_seq: cctttagagtaaacccggccatc; mol_type = other DNA; note = ORF 39
gcgggagagccaatctgatgcctttagagtaaacccggccatc

SEQ ID NO: 49 - DNA, 42 bp, synthetic construct
  source 1..42: PCR_primers = fwd_seq: aggtgagacctactgtccctg, rev_seq:ggtgatgtgacgtgggttagg; mol_type = other DNA; note = ORF 40
aggtgagacctactgtccctgggtgatgtgacgtgggttagg

SEQ ID NO: 50 - DNA, 43 bp, synthetic construct
  source 1..43: PCR_primers = fwd_seq: gatcgcggacctgcttcagatg, rev_seq:tctaagtggcccatcacggac; mol_type = other DNA; note = ORF 41
gatcgcggacctgcttcagatgtctaagtggcccatcacggac

SEQ ID NO: 51 - DNA, 46 bp, synthetic construct
  source 1..46: PCR_primers = fwd_seq: cttcgtcttccagtggatcga, rev_seq:ataagaatacttgccttgcaggatc; mol_type = other DNA; note = ORF 42
cttcgtcttccagtggatcgaataagaatacttgccttgcaggatc

SEQ ID NO: 52 - DNA, 44 bp, synthetic construct
  source 1..44: PCR_primers = fwd_seq: actcgtatgtcctccagtcg, rev_seq:caacctgtcaatctgttccactac; mol_type = other DNA; note = ORF 43
actcgtatgtcctccagtcgcaacctgtcaatctgttccactac

SEQ ID NO: 53 - DNA, 46 bp, synthetic construct
  source 1..46: PCR_primers = fwd_seq: actctgatctactgacccgtacc, rev_seq: ccaacgactatttgactcgccac; mol_type = other DNA; note = ORF 44
actctgatctactgacccgtaccccaacgactatttgactcgccac

SEQ ID NO: 54 - DNA, 48 bp, synthetic construct
  source 1..48: PCR_primers = fwd_seq:tggaagcattctctcttcatcgtg, rev_seq: cgaagtttgacggcctatactgta; mol_type = other DNA; note = ORF 45
tggaagcattctctcttcatcgtgcgaagtttgacggcctatactgta

SEQ ID NO: 55 - DNA, 41 bp, synthetic construct
  source 1..41: PCR_primers = fwd_seq: ctgagcagcgagagcagtttc, rev_seq: cctcattagtcgggactcgc; mol_type = other DNA; note = ORF 46
ctgagcagcgagagcagtttccctcattagtcgggactcgc

SEQ ID NO: 56 - DNA, 40 bp, synthetic construct
  source 1..40: PCR_primers = fwd_seq:gcgagtcccgactaatgagg, rev_seq: gtcattgccacccagctact; mol_type = other DNA; note = ORF 47
gcgagtcccgactaatgagggtcattgccacccagctact

SEQ ID NO: 57 - DNA, 46 bp, synthetic construct
  source 1..46: PCR_primers = fwd_seq:cagtagctgggtggcaatgac, rev_seq: ggtcactgggatcgtagattgtttc; mol_type = other DNA; note = ORF 48
cagtagctgggtggcaatgacggtcactgggatcgtagattgtttc

SEQ ID NO: 58 - DNA, 46 bp, synthetic construct; identical to SEQ ID NO: 57
  source 1..46: PCR_primers = fwd_seq:cagtagctgggtggcaatgac, rev_seq: ggtcactgggatcgtagattgtttc; mol_type = other DNA; note = ORF 49

SEQ ID NO: 59 - DNA, 42 bp, synthetic construct
  source 1..42: PCR_primers = fwd_seq: cacgaatctggttgattgtgac, rev_seq: ctgttccttatgtgcctcca; mol_type = other DNA; note = ORF 50
cacgaatctggttgattgtgacctgttccttatgtgcctcca

SEQ ID NO: 60 - DNA, 43 bp, synthetic construct
  source 1..43: PCR_primers = fwd_seq: gggagaaccatgccagactttg, rev_seq:catcgtggaacgcacaggtaa; mol_type = other DNA; note = ORF K8
gggagaaccatgccagactttgcatcgtggaacgcacaggtaa

SEQ ID NO: 61 - DNA, 42 bp, synthetic construct
  source 1..42: PCR_primers = fwd_seq:gcgtaagaaaccctacatagtg , rev_seq:gcatagactggcatgtgatt; mol_type = other DNA; note = ORF K8.1
gcgtaagaaaccctacatagtggcatagactggcatgtgatt

SEQ ID NO: 62 - DNA, 47 bp, synthetic construct
  source 1..47: PCR_primers = fwd_seq: gtagatgtacgtgttggtgatgctc, rev_seq: gtctggcttcatttgctctcga; mol_type = other DNA; note = ORF 52
gtagatgtacgtgttggtgatgctcgtctggcttcatttgctctcga

SEQ ID NO: 63 - DNA, 44 bp, synthetic construct
  source 1..44: PCR_primers = fwd_seq:tactgggactagaacgctctg, rev_seq: gataggttcgacataggttggct; mol_type = other DNA; note = ORF 53
tactgggactagaacgctctggataggttcgacataggttggct

SEQ ID NO: 64 - DNA, 40 bp, synthetic construct
  source 1..40: PCR_primers = fwd_seq: gctgtcgccactcgtacaaa, rev_seq:gtgccaaggttcgactggac; mol_type = other DNA; note = ORF 54
gctgtcgccactcgtacaaagtgccaaggttcgactggac

SEQ ID NO: 65 - DNA, 40 bp, synthetic construct
  source 1..40: PCR_primers = fwd_seq:acgtcacccagacacactcc, rev_seq: gctgtcgccactcgtacaaa; mol_type = other DNA; note = ORF 55
acgtcacccagacacactccgctgtcgccactcgtacaaa

SEQ ID NO: 66 - DNA, 40 bp, synthetic construct; identical to SEQ ID NO: 65
  source 1..40: PCR_primers = fwd_seq: acgtcacccagacacactcc, rev_seq: gctgtcgccactcgtacaaa; mol_type = other DNA; note = ORF 56

SEQ ID NO: 67 - DNA, 43 bp, synthetic construct
  source 1..43: PCR_primers = fwd_seq: caccttaaacacaacaccagacc, rev_seq:ccaggcaattctgcggctag; mol_type = other DNA; note = ORF 57
caccttaaacacaacaccagaccccaggcaattctgcggctag

SEQ ID NO: 68 - DNA, 43 bp, synthetic construct
  source 1..43: PCR_primers = fwd_seq: tgaatggtaactgtctggacac, rev_seq:gagaacaaagctacgaggagg; mol_type = other DNA; note = ORF K9
tgaatggtaactgtctggacacgagaacaaagctacgaggagg

SEQ ID NO: 69 - DNA, 42 bp, synthetic construct
  source 1..42: PCR_primers = fwd_seq: ctcttgacctggtaaccctgg, rev_seq: cagttgatgatgccaatgccg; mol_type = other DNA; note = ORF K10
ctcttgacctggtaaccctggcagttgatgatgccaatgccg

SEQ ID NO: 70 - DNA, 44 bp, synthetic construct
  source 1..44: PCR_primers = fwd_seq: gtcgccacgcccacaacatc, rev_seq:cccgttggcaaacatagatccgtc; mol_type = other DNA; note = ORF K10.5
gtcgccacgcccacaacatccccgttggcaaacatagatccgtc

SEQ ID NO: 71 - DNA, 46 bp, synthetic construct
  source 1..46: PCR_primers = fwd_seq: gcactgtccaccctctaatacaag, rev_seq: ctcacaccagttggtccctttg; mol_type = other DNA; note = ORF K11
gcactgtccaccctctaatacaagctcacaccagttggtccctttg

SEQ ID NO: 72 - DNA, 49 bp, synthetic construct
  source 1..49: PCR_primers = fwd_seq: ggtaaagagtgtgaacgagtacagg, rev_seq:gtgtgactgacgatttgtgaaggt; mol_type = other DNA; note = ORF 58
ggtaaagagtgtgaacgagtacagggtgtgactgacgatttgtgaaggt

SEQ ID NO: 73 - DNA, 42 bp, synthetic construct
  source 1..42: PCR_primers = fwd_seq: gccgtagacgcacagagaaatc, rev_seq:cttcagtgcctggcagatcc; mol_type = other DNA; note = ORF 59
gccgtagacgcacagagaaatccttcagtgcctggcagatcc

SEQ ID NO: 74 - DNA, 44 bp, synthetic construct
  source 1..44: PCR_primers = fwd_seq: cgtgggaaacatcaaggtgc, rev_seq: gcacagttccctttgattctcatc; mol_type = other DNA; note = ORF 60
cgtgggaaacatcaaggtgcgcacagttccctttgattctcatc

SEQ ID NO: 75 - DNA, 43 bp, synthetic construct
  source 1..43: PCR_primers = fwd_seq:gtctatgagagattgggcacac, rev_seq: gctctgttgtgctgctgttta; mol_type = other DNA; note = ORF 61
gtctatgagagattgggcacacgctctgttgtgctgctgttta

SEQ ID NO: 76 - DNA, 44 bp, synthetic construct
  source 1..44: PCR_primers = fwd_seq: gtggacgccgcatatttagagag, rev_seq:ggtacatgacgcagttgctga; mol_type = other DNA; note = ORF 62
gtggacgccgcatatttagagagggtacatgacgcagttgctga

SEQ ID NO: 77 - DNA, 43 bp, synthetic construct
  source 1..43: PCR_primers = fwd_seq: gaaacgtactcccggtctgc, rev_seq: gtataaccaccctgtcctctggt; mol_type = other DNA; note = ORF 63
gaaacgtactcccggtctgcgtataaccaccctgtcctctggt

SEQ ID NO: 78 - DNA, 42 bp, synthetic construct
  source 1..42: PCR_primers = fwd_seq: ggtcacccatagtacccatcag, rev_seq: ctgcgaggctgccctattaa; mol_type = other DNA; note = ORF 64
ggtcacccatagtacccatcagctgcgaggctgccctattaa

SEQ ID NO: 79 - DNA, 42 bp, synthetic construct
  source 1..42: PCR_primers = fwd_seq:gtcacagcggtatattgggc, rev_seq: aaagccacatattcctccactg; mol_type = other DNA; note = ORF 65
gtcacagcggtatattgggcaaagccacatattcctccactg

SEQ ID NO: 80 - DNA, 45 bp, synthetic construct
  source 1..45: PCR_primers = fwd_seq: gttggagagcaaggtggacacg, rev_seq: accgtgctgcattctaaccgtac; mol_type = other DNA; note = ORF 66
gttggagagcaaggtggacacgaccgtgctgcattctaaccgtac

SEQ ID NO: 81 - DNA, 43 bp, synthetic construct
  source 1..43: PCR_primers = fwd_seq: gagagttggaagagacgcggg, rev_seq: ccaccttgctctccaacaccag; mol_type = other DNA; note = ORF ORF 67
gagagttggaagagacgcgggccaccttgctctccaacaccag

SEQ ID NO: 82 - DNA, 43 bp, synthetic construct
  source 1..43: PCR_primers = fwd_seq: ccaccttgctctccaacaccag, rev_seq: gagagttggaagagacgcggg; mol_type = other DNA
ccaccttgctctccaacaccaggagagttggaagagacgcggg

SEQ ID NO: 83 - DNA, 44 bp, synthetic construct
  source 1..44: PCR_primers = fwd_seq: ttattcgggagctaaccgcac, rev_seq: cacctttcatggcagtacattgc; mol_type = other DNA; note = ORF 68
ttattcgggagctaaccgcaccacctttcatggcagtacattgc

SEQ ID NO: 84 - DNA, 44 bp, synthetic construct; identical to SEQ ID NO: 83
  source 1..44: PCR_primers = fwd_seq: ttattcgggagctaaccgcac, rev_seq: cacctttcatggcagtacattgc; mol_type = other DNA; note = ORF 69

SEQ ID NO: 85 - DNA, 43 bp, synthetic construct
  source 1..43: PCR_primers = fwd_seq: tcgccttcaaacagaagcacg, rev_seq: gatgtttccgttctacaggcgg; mol_type = other DNA; note = ORF K12
tcgccttcaaacagaagcacggatgtttccgttctacaggcgg

SEQ ID NO: 86 - DNA, 42 bp, synthetic construct
  source 1..42: PCR_primers = fwd_seq:tgtcatccgtgcccagtttc, rev_seq: gttctcacgacccatctacctc; mol_type = other DNA; note = ORFK13
tgtcatccgtgcccagtttcgttctcacgacccatctacctc

SEQ ID NO: 87 - DNA, 40 bp, synthetic construct
  source 1..40: PCR_primers = fwd_seq: atgggtcgtgagaacactgc, rev_seq: ctgcgatctccatcctgtgg; mol_type = other DNA; note = ORF 72
atgggtcgtgagaacactgcctgcgatctccatcctgtgg

SEQ ID NO: 88 - DNA, 43 bp, synthetic construct
  source 1..43: PCR_primers = fwd_seq:aaactgaagaaggcgtgtctgc , rev_seq:gaagtgactgccaaaccacac; mol_type = other DNA; note = ORF 73
aaactgaagaaggcgtgtctgcgaagtgactgccaaaccacac

SEQ ID NO: 89 - DNA, 45 bp, synthetic construct
  source 1..45: PCR_primers = fwd_seq: tgtgttgaaggacggatcagg, rev_seq:caagaagatcaacgaccaccacta; mol_type = other DNA; note = ORF K14
tgtgttgaaggacggatcaggcaagaagatcaacgaccaccacta

SEQ ID NO: 90 - DNA, 45 bp, synthetic construct; identical to SEQ ID NO: 89
  source 1..45: PCR_primers = fwd_seq: tgtgttgaaggacggatcagg, rev_seq: caagaagatcaacgaccaccacta; mol_type = other DNA; note = ORF 74

SEQ ID NO: 91 - DNA, 45 bp, synthetic construct
  source 1..45: PCR_primers = fwd_seq: tctagcctcccgttcccatg, rev_seq: aaagccctaacccaagtctgactac; mol_type = other DNA; note = ORF 75
tctagcctcccgttcccatgaaagccctaacccaagtctgactac

SEQ ID NO: 92 - DNA, 47 bp, synthetic construct
  source 1..47: PCR_primers = fwd_seq: gagccttgtgtcgggaatacttag, rev_seq:tattacgcaggcacaggttgctc; mol_type = other DNA; note = ORF K15
gagccttgtgtcgggaatacttagtattacgcaggcacaggttgctc

SEQ ID NO: 93 - DNA, 82 bp, synthetic construct
  source 1..82: mol_type = other DNA; note = Forward sequence ORF K1
cctgtctttcagaccttgttggacatcccgtacaatcaagcattcaggtaagataatctaaggatgacgacgataagtaggg

SEQ ID NO: 94 - DNA, 84 bp, synthetic construct
  source 1..84: mol_type = other DNA; note = Reverse sequence ORF K1
attatgttatagagaatatttagattatcttacctgaatgcttgattgtacgggatgtccaaccaattaaccaattctgattag

SEQ ID NO: 95 - DNA, 82 bp, synthetic construct
  source 1..82: mol_type = other DNA; note = Forward sequence ORF 2
ctaacgcggcatacactagccggtggtgcccgagcgggaggccgcgagggtataggtaaaaggatgacgacgataagtaggg

SEQ ID NO: 96 - DNA, 84 bp, synthetic construct
  source 1..84: mol_type = other DNA; note = Reverse sequence ORF 2
acactgtgtggttggtggtgtttacctataccctcgcggcctcccgctcgggcaccaccgaaccaattaaccaattctgattag

SEQ ID NO: 97 - DNA, 82 bp, synthetic construct
  source 1..82: mol_type = other DNA; note = Forward sequence ORF 4
gtacattaaaaggacattgtataaccgtgctacttacagccctagacttgctccagtgttaggatgacgacgataagtaggg

SEQ ID NO: 98 - DNA, 84 bp, synthetic construct
  source 1..84: mol_type = other DNA; note = Reverse sequence ORF 4
aagcaatcatagccctgtctaacactggagcaagtctagggctgtaagtagcacggttataaccaattaaccaattctgattag

SEQ ID NO: 99 - DNA, 82 bp, synthetic construct
  source 1..82: mol_type = other DNA; note = Forward sequence ORF 6
tacacacgggttttttgttgtcttggccaatcgtgtctccttgtgtacccgtaacgatggaggatgacgacgataagtaggg

SEQ ID NO: 100 - DNA, 84 bp, synthetic construct
  source 1..84: mol_type = other DNA; note = Reverse sequence ORF 6
gaccgccgccagttcctttgccatcgttacgggtacacaaggagacacgattggccaagaaaccaattaaccaattctgattag

SEQ ID NO: 101 - DNA, 94 bp, synthetic construct
  source 1..94: mol_type = other DNA; note = Forward sequence ORF 7
gacctggatttgtagttgtgtacccgtaacgatggcaaagtaggtagatagggaactggcggcggtctatgcaggatgacgacgataagtaggg

SEQ ID NO: 102 - DNA, 96 bp, synthetic construct
  source 1..96: mol_type = other DNA; note = Reverse sequence ORF 7
tggctagggctgacacatcggcatagaccgccgccagttccctatctacctactttgccatcgttacgggtaaaccaattaaccaattctgattag

SEQ ID NO: 103 - DNA, 94 bp, synthetic construct
  source 1..94: mol_type = other DNA; note = Forward sequence ORF 8
ctgtaccaccacctgcaattgagcaaccacaatgactccctaggtagataggaggtctagattggccaccctaggatgacgacgataagtaggg

SEQ ID NO: 104 - DNA, 96 bp, synthetic construct
  source 1..96: mol_type = other DNA; note = Reverse sequence ORF 8
ccaacaggatgacagtccccagggtggccaatctagacctcctatctacctagggagtcattgtggttgctcaaccaattaaccaattctgattag

SEQ ID NO: 105 - DNA, 82 bp, synthetic construct
  source 1..82: mol_type = other DNA; note = Forward sequence ORF 9
ctactcgttacacacagacacaaattaccgtccgcagatctgactcagacgcggaaacagaggatgacgacgataagtaggg

SEQ ID NO: 106 - DNA, 84 bp, synthetic construct
  source 1..84: mol_type = other DNA; note = Reverse sequence ORF 9
aagaggaaactttctaggcgctgtttccgcgtctgagtcagatctgcggacggtaatttgaaccaattaaccaattctgattag

SEQ ID NO: 107 - DNA, 94 bp, synthetic construct
  source 1..94: mol_type = other DNA; note = Forward sequence ORF 10.1
cttgcgctatgtgggacaactagagtccaacctggcaagctaggtagataggagtggagcaagacgccagacaggatgacgacgataagtaggg

SEQ ID NO: 108 - DNA, 96 bp, synthetic construct
  source 1..96: mol_type = other DNA; note = Reverse sequence ORF 10.1
tatttttttcgagatcggctgtctggcgtcttgctccactcctatctacctagcttgccaggttggactctaaaccaattaaccaattctgattag

SEQ ID NO: 109 - DNA, 94 bp, synthetic construct
  source 1..94: mol_type = other DNA; note = Forward sequence ORF 10
aacgttcatcctaggtgactgggagataacggtgtctaactaggtagataggtgccggtttacttgcagcagaggatgacgacgataagtaggg

SEQ ID NO: 110 - DNA, 96 bp, synthetic construct
  source 1..96: mol_type = other DNA; note = Reverse sequence ORF 10
aaagggggccacatgttaggctgctgcaagtaaaccggcacctatctacctagttagacaccgttatctcccaaccaattaaccaattctgattag

SEQ ID NO: 111 - DNA, 94 bp, synthetic construct
  source 1..94: mol_type = other DNA; note = Forward sequence ORF 11AA
ccacgtagcgattagggccgaccgccacgaggaacccatgtaggtagataggcaatcgtgactgtccgagcaaggatgacgacgataagtaggg

SEQ ID NO: 112 - DNA, 96 bp, synthetic construct
  source 1..96: mol_type = other DNA; note = Reverse sequence ORF 11AA
tctgactcctgcgccatatgtgctcggacagtcacgattgcctatctacctacatgggttcctcgtggcggtaaccaattaaccaattctgattag

SEQ ID NO: 113 - DNA, 82 bp, synthetic construct
  source 1..82: mol_type = other DNA; note = Forward sequence ORF 11
gccacgaggaacccatgcaatcgtgactgtccgagcacatgtgtccggttcccacccacaaggatgacgacgataagtaggg

SEQ ID NO: 114 - DNA, 84 bp, synthetic construct
  source 1..84: mol_type = other DNA; note = Reverse sequence ORF 11
gaaagcaataaagacaaatgtgtgggtgggaaccggacacatgtgctcggacagtcacgaaaccaattaaccaattctgattag

SEQ ID NO: 115 - DNA, 82 bp, synthetic construct
  source 1..82: mol_type = other DNA; note = Forward sequence ORF K2
gtatattagtgttataagaaattttatgtcacgtcgctctggctgctaacgcggcatacaaggatgacgacgataagtaggg

SEQ ID NO: 116 - DNA, 84 bp, synthetic construct
  source 1..84: mol_type = other DNA; note = Reverse sequence ORF K2
gctcgggcaccaccggctagtgtatgccgcgttagcagccagagcgacgtgacataaaataaccaattaaccaattctgattag

SEQ ID NO: 117 - DNA, 82 bp, synthetic construct; identical to SEQ ID NO: 95
  source 1..82: mol_type = other DNA; note = Forward sequence ORF 2

SEQ ID NO: 118 - DNA, 84 bp, synthetic construct; identical to SEQ ID NO: 96
  source 1..84: mol_type = other DNA; note = Reverse sequence ORF 2

SEQ ID NO: 119 - DNA, 82 bp, synthetic construct
  source 1..82: mol_type = other DNA; note = Forward sequence ORF K3
ggtaaacaccaccaaccacacagtgtgctcttatatacttatcctgagagagaacccacaaggatgacgacgataagtaggg

SEQ ID NO: 120 - DNA, 84 bp, synthetic construct
  source 1..84: mol_type = other DNA; note = Reverse sequence ORF K3
gggttaatgccatgttttattgtgggttctctctcaggataagtatataagagcacactgaaccaattaaccaattctgattag

SEQ ID NO: 121 - DNA, 82 bp, synthetic construct
  source 1..82: mol_type = other DNA; note = Forward sequence ORF 70
gcccaccactctgaccgcacgctaaacatcgccctacctggatagatcctggaagtttgtaggatgacgacgataagtaggg

SEQ ID NO: 122 - DNA, 84 bp, synthetic construct
  source 1..84: mol_type = other DNA; note = Reverse sequence ORF 70
ctctccgggcacagggcttcacaaacttccaggatctatccaggtagggcgatgtttagcaaccaattaaccaattctgattag

SEQ ID NO: 123 - DNA, 82 bp, synthetic construct
  source 1..82: mol_type = other DNA; note = Forward sequence ORF K4
agagatccgtcgcgtaaatgcgcagctggcaaagcattctaactcccctcgtgtgtcctcaggatgacgacgataagtaggg

SEQ ID NO: 124 - DNA, 84 bp, synthetic construct
  source 1..84: mol_type = other DNA; note = Reverse sequence ORF K4
tcgccgtttcgcatttacacgaggacacacgaggggagttagaatgctttgccagctgcgaaccaattaaccaattctgattag

SEQ ID NO: 125 - DNA, 82 bp, synthetic construct
  source 1..82: mol_type = other DNA; note = Forward sequence ORF K 4.1
tatgtcacagactcaacacacacgggccgttacgcaacggacagttctggcgccacaacgaggatgacgacgataagtaggg

SEQ ID NO: 126 - DNA, 84 bp, synthetic construct
  source 1..84: mol_type = other DNA; note = Reverse sequence ORF K4.1
tgcacccctttgcgcatcatcgttgtggcgccagaactgtccgttgcgtaacggcccgtgaaccaattaaccaattctgattag

SEQ ID NO: 127 - DNA, 94 bp, synthetic construct
  source 1..94: mol_type = other DNA; note = Forward sequence ORF K4.2
acataatttatgcacataaaaggattagcgcatgcaaatttaggtagataggagctttgccgaagttctcggaggatgacgacgataagtaggg

SEQ ID NO: 128 - DNA, 96 bp, synthetic construct
  source 1..96: mol_type = other DNA; note = Reverse sequence ORF K4.2
cagcgcgccccaccggctttccgagaacttcggcaaagctcctatctacctaaatttgcatgcgctaatcctaaccaattaaccaattctgattag

SEQ ID NO: 129 - DNA, 82 bp, synthetic construct
  source 1..82: mol_type = other DNA; note = Forward sequence ORF K5
gggcgtcacgtcacatatctctgtgcacccaagtggttgtctctgcagctggggtggaagaggatgacgacgataagtaggg

SEQ ID NO: 130 - DNA, 84 bp, synthetic construct
  source 1..84: mol_type = other DNA; note = Reverse sequence ORF K5
tcccctttccctttttcagacttccaccccagctgcagagacaaccacttgggtgcacagaaccaattaaccaattctgattag

SEQ ID NO: 131 - DNA, 82 bp, synthetic construct
  source 1..82: mol_type = other DNA; note = Forward sequence ORF K6
gagcagttgggccgcagtgatatcttcaactttcgaccgtctggaggtgccaagttcgcaaggatgacgacgataagtaggg

SEQ ID NO: 132 - DNA, 84 bp, synthetic construct
  source 1..84: mol_type = other DNA; note = Reverse sequence ORF K6
tacggttttctttagactgttgcgaacttggcacctccagacggtcgaaagttgaagataaaccaattaaccaattctgattag

SEQ ID NO: 133 - DNA, 94 bp, synthetic construct
  source 1..94: mol_type = other DNA; note = Forward sequence ORF K7
tccaaaaatgggtggctaacctgtccaaaatatgggaacataggtagataggctggagataaaaggggccagaggatgacgacgataagtaggg

SEQ ID NO: 134 - DNA, 96 bp, synthetic construct
  source 1..96: mol_type = other DNA; note = Reverse sequence ORF K7
cagtgctaaactgactcaagctggccccttttatctccagcctatctacctatgttcccatattttggacagaaccaattaaccaattctgattag

SEQ ID NO: 135 - DNA, 82 bp, synthetic construct
  source 1..82: mol_type = other DNA; note = Forward sequence ORF 16
ggtgctgtgcgcgtgctatgttccctggtgaccgtccacacgcgtaattcgaggtccccgaggatgacgacgataagtaggg

SEQ ID NO: 136 - DNA, 84 bp, synthetic construct
  source 1..84: mol_type = other DNA; note = Reverse sequence ORF 16
catgcaaccatctactcttccggggacctcgaattacgcgtgtggacggtcaccagggaaaaccaattaaccaattctgattag

SEQ ID NO: 137 - DNA, 94 bp, synthetic construct
  source 1..94: mol_type = other DNA; note = Forward sequence ORF 17
ctcggtctcacacacgtattttccgagcatggcacagggctaggtagataggctgtacgtcggagggtttgtaggatgacgacgataagtaggg

SEQ ID NO: 138 - DNA, 96 bp, synthetic construct
  source 1..96: mol_type = other DNA; note = Reverse sequence ORF 17
tggggcaggacacaacatctacaaaccctccgacgtacagcctatctacctagccctgtgccatgctcggaaaaccaattaaccaattctgattag

SEQ ID NO: 139 - DNA, 94 bp, synthetic construct
  source 1..94: mol_type = other DNA; note = Forward sequence ORF 18
gccgctgagcccggggcttaggaggctcatgtggcgcttttaggtagataggttgcaaaataagaatttaaaaggatgacgacgataagtaggg

SEQ ID NO: 140 - DNA, 96 bp, synthetic construct
  source 1..96: mol_type = other DNA; note = Reverse sequence ORF 18
gctcttgggcgtggaatgtatttaaattcttattttgcaacctatctacctaaaagcgccacatgagcctccaaccaattaaccaattctgattag

SEQ ID NO: 141 - DNA, 94 bp, synthetic construct
  source 1..94: mol_type = other DNA; note = Forward sequence ORF 19
ggaccggcttgttcaggtccatgactcacgcgtccgcgtctaggtagataggattaacgcagatatcgacgcaggatgacgacgataagtaggg

SEQ ID NO: 142 - DNA, 96 bp, synthetic construct
  source 1..96: mol_type = other DNA; note = Reverse sequence ORF 19
tgcctatcatctgtttcaccgcgtcgatatctgcgttaatcctatctacctagacgcggacgcgtgagtcataaccaattaaccaattctgattag

SEQ ID NO: 143 - DNA, 94 bp, synthetic construct
  source 1..94: mol_type = other DNA; note = Forward sequence ORF 20
cgagtccgctccaaaaccgccttctgccatggtacgtccataggtagataggaccgaggccgaggttaagaaaggatgacgacgataagtaggg

SEQ ID NO: 144 - DNA, 96 bp, synthetic construct
  source 1..96: mol_type = other DNA; note = Reverse sequence ORF 20
ctggaagcctgctcagggatttcttaacctcggcctcggtcctatctacctatggacgtaccatggcagaagaaccaattaaccaattctgattag

SEQ ID NO: 145 - DNA, 94 bp, synthetic construct
  source 1..94: mol_type = other DNA; note = Forward sequence ORF 21
tttcttaacctcggcctcggttggacgtaccatggcagaataggtagataggggcggttttggagcggactcaggatgacgacgataagtaggg

SEQ ID NO: 146 - DNA, 96 bp, synthetic construct
  source 1..96: mol_type = other DNA; note = Reverse sequence ORF 21
tttctccgccgcgccccaccgagtccgctccaaaaccgcccctatctacctattctgccatggtacgtccaaaaccaattaaccaattctgattag

SEQ ID NO: 147 - DNA, 94 bp, synthetic construct
  source 1..94: mol_type = other DNA; note = Forward sequence ORF 22
agtctaaagcagttaatcacctagaggagacatgcagggttaggtagataggctagccttcttggcggccctaggatgacgacgataagtaggg

SEQ ID NO: 148 - DNA, 96 bp, synthetic construct
  source 1..96: mol_type = other DNA; note = Reverse sequence ORF 22
atatgcatcgccagcatgcaagggccgccaagaaggctagcctatctacctaaccctgcatgtctcctctagaaccaattaaccaattctgattag

SEQ ID NO: 149 - DNA, 94 bp, synthetic construct
  source 1..94: mol_type = other DNA; note = Forward sequence ORF 23
cgtacgttgcgtccgtccgctggtctaagctatgttacgataggtagatagggttccggacgtgaaggctagaggatgacgacgataagtaggg

SEQ ID NO: 150 - DNA, 96 bp, synthetic construct
  source 1..96: mol_type = other DNA; note = Reverse sequence ORF 23
gcgccgcgccctctactagactagccttcacgtccggaaccctatctacctatcgtaacatagcttagaccaaaccaattaaccaattctgattag

SEQ ID NO: 151 - DNA, 94 bp, synthetic construct
  source 1..94: mol_type = other DNA; note = Forward sequence ORF 24
cgggaaaggtcgttgctccaaggtcgcctccatggcagcgtaggtagataggctcgagggccccctactactaggatgacgacgataagtaggg

SEQ ID NO: 152 - DNA, 96 bp, synthetic construct
  source 1..96: mol_type = other DNA; note = Reverse sequence ORF 24
tcagggaggcgctcggtggcagtagtagggggccctcgagcctatctacctacgctgccatggaggcgacctaaccaattaaccaattctgattag

SEQ ID NO: 153 - DNA, 94 bp, synthetic construct
  source 1..94: mol_type = other DNA; note = Forward sequence ORF 25
tggcagtagtagggggccctcgagcgctgccatggaggcgtaggtagataggaccttggagcaacgacctttaggatgacgacgataagtaggg

SEQ ID NO: 154 - DNA, 96 bp, synthetic construct
  source 1..96: mol_type = other DNA; note = Reverse sequence ORF 25
cctccgtggcgaggtacgggaaaggtcgttgctccaaggtcctatctacctacgcctccatggcagcgctcgaaccaattaaccaattctgattag

SEQ ID NO: 155 - DNA, 94 bp, synthetic construct
  source 1..94: mol_type = other DNA; note = Forward sequence ORF 26
tattagctaacccttctagcgttggctagtcatggcactctaggtagatagggacaagagtatagtggttaaaggatgacgacgataagtaggg

SEQ ID NO: 156 - DNA, 96 bp, synthetic construct
  source 1..96: mol_type = other DNA; note = Reverse sequence ORF 26
cgaagagtctggaggtgaagttaaccactatactcttgtccctatctacctagttggctagtcatggcactcaaccaattaaccaattctgattag

SEQ ID NO: 157 - DNA, 94 bp, synthetic construct
  source 1..94: mol_type = other DNA; note = Forward sequence ORF 27
gagactttggcggcctcctgttggtattccccacgctaactaggtagatagggatttgaagcggggggggggaggatgacgacgataagtaggg

SEQ ID NO: 158 - DNA, 96 bp, synthetic construct
  source 1..96: mol_type = other DNA; note = Reverse sequence ORF 27
gaatatcagatgacgccataccccccccccgcttcaaatccctatctacctagttagcgtggggaataccaaaaccaattaaccaattctgattag

SEQ ID NO: 159 - DNA, 82 bp, synthetic construct
  source 1..82: mol_type = other DNA; note = Forward sequence ORF 28
ctcagttgagagtcagagaatacagtgctaatcagggtagaacggggtgtgtgctataataggatgacgacgataagtaggg

SEQ ID NO: 160 - DNA, 84 bp, synthetic construct
  source 1..84: mol_type = other DNA; note = Reverse sequence ORF 28
tacagtgctaatcagggtagagccccccccatagccatccattatagcacacaccccgttaaccaattaaccaattctgattag

SEQ ID NO: 161 - DNA, 94 bp, synthetic construct
  source 1..94: mol_type = other DNA; note = Forward sequence ORF 29b
aaaggatgcactgccggctattctgggtttcatgcttcagtaggtagataggaaagacgccaagcttatattaggatgacgacgataagtaggg

SEQ ID NO: 162 - DNA, 96 bp, synthetic construct
  source 1..96: mol_type = other DNA; note = Reverse sequence ORF 29b
acgagttcacggatgatataaatataagcttggcgtctttcctatctacctactgaagcatgaaacccagaaaaccaattaaccaattctgattag

SEQ ID NO: 163 - DNA, 94 bp, synthetic construct
  source 1..94: mol_type = other DNA; note = Forward sequence ORF 30.1
tacgtagagcaggttaaaggtctgtccccgaatgctctgctaggtagataggagacacggaaagacacaaaaaggatgacgacgataagtaggg

SEQ ID NO: 164 - DNA, 96 bp, synthetic construct
  source 1..96: mol_type = other DNA; note = Reverse sequence ORF 30.1
tagccgcttatgagcccctcttttgtgtctttccgtgtctcctatctacctagcagagcattcggggacagaaaccaattaaccaattctgattag

SEQ ID NO: 165 - DNA, 94 bp, synthetic construct
  source 1..94: mol_type = other DNA; note = Forward sequence ORF 30
gggaataaaagggggcgtgtgtgccgatcgtatgggtgagtaggtagataggccagtggatcctggacatgtaggatgacgacgataagtaggg

SEQ ID NO: 166 - DNA, 96 bp, synthetic construct
  source 1..96: mol_type = other DNA; note = Reverse sequence ORF 30
caaaatctttctcattcaccacatgtccaggatccactggcctatctacctagtgccgatcgtatgggtgagaaccaattaaccaattctgattag

SEQ ID NO: 167 - DNA, 94 bp, synthetic construct
  source 1..94: mol_type = other DNA; note = Forward sequence ORF 31
tgtgccgcctagacaccggtgcgaaatgaagagtgtggcgtaggtagataggagtcccttatgtcagttccaaggatgacgacgataagtaggg

SEQ ID NO: 168 - DNA, 96 bp, synthetic construct
  source 1..96: mol_type = other DNA; note = Reverse sequence ORF 31
ggtacaggcaaaacacgccgtggaactgacataagggactcctatctacctacgccacactcttcatttcgcaaccaattaaccaattctgattag

SEQ ID NO: 169 - DNA, 94 bp, synthetic construct
  source 1..94: mol_type = other DNA; note = Forward sequence ORF 32
acgactgcattgccaagcgggtgcggacaaaatggatgcgtaggtagataggcatgctatcaacgaaagataaggatgacgacgataagtaggg

SEQ ID NO: 170 - DNA, 96 bp, synthetic construct
  source 1..96: mol_type = other DNA; note = Reverse sequence ORF 32
ggtggcagcgaggacctacgtatctttcgttgatagcatgcctatctacctagtgcggacaaaatggatgcgaaccaattaaccaattctgattag

SEQ ID NO: 171 - DNA, 94 bp, synthetic construct
  source 1..94: mol_type = other DNA; note = Forward sequence ORF 33
aaggaggatctggtgttcattcgaggccgctatggctagctaggtagataggcggaggcgcaaacttcggaaaggatgacgacgataagtaggg

SEQ ID NO: 172 - DNA, 96 bp, synthetic construct
  source 1..96: mol_type = other DNA; note = Reverse sequence ORF 33
tgcattccttgtttaggaaattccgaagtttgcgcctccgcctatctacctagctagccatagcggcctcgaaaccaattaaccaattctgattag

SEQ ID NO: 173 - DNA, 94 bp, synthetic construct
  source 1..94: mol_type = other DNA; note = Forward sequence ORF 29a
accctcggacacgagcgagctcaaagcaaacatgctgctctaggtagataggagccgtcacagggagcgcctaggatgacgacgataagtaggg

SEQ ID NO: 174 - DNA, 96 bp, synthetic construct
  source 1..96: mol_type = other DNA; note = Reverse sequence ORF 29a
tctcctgcaggttggcggcaaggcgctccctgtgacggctcctatctacctagagcagcatgtttgctttgaaaccaattaaccaattctgattag

SEQ ID NO: 175 - DNA, 94 bp, synthetic construct
  source 1..94: mol_type = other DNA; note = Forward sequence ORF 34.1
atccgtgccgttttgggacagtgtcgcgtgaatgtcggggtaggtagataggcactcagttcccacctctctaggatgacgacgataagtaggg

SEQ ID NO: 176 - DNA, 96 bp, synthetic construct
  source 1..96: mol_type = other DNA; note = Reverse sequence ORF 34.1
gagaccgccaaagacgccggagagaggtgggaactgagtgcctatctacctaccccgacattcacgcgacacaaccaattaaccaattctgattag

SEQ ID NO: 177 - DNA, 94 bp, synthetic construct
  source 1..94: mol_type = other DNA; note = Forward sequence ORF 34
ggcaaggcgctccctgtgacggctgagcagcatgtttgcttaggtagataggttgagctcgctcgtgtccgaaggatgacgacgataagtaggg

SEQ ID NO: 178 - DNA, 96 bp, synthetic construct
  source 1..96: mol_type = other DNA; note = Reverse sequence ORF 34
tggtcacctccgggtcaccctcggacacgagcgagctcaacctatctacctaagcaaacatgctgctcagccaaccaattaaccaattctgattag

SEQ ID NO: 179 - DNA, 94 bp, synthetic construct
  source 1..94: mol_type = other DNA; note = Forward sequence ORF 35
ataccggcgatcatcaccatgatcaaggagaatggactcataggtagataggaccaactctaaaagagagttaggatgacgacgataagtaggg

SEQ ID NO: 180 - DNA, 96 bp, synthetic construct
  source 1..96: mol_type = other DNA; note = Reverse sequence ORF 35
cctccagagccgacttaataaactctcttttagagttggtcctatctacctatgagtccattctccttgatcaaccaattaaccaattctgattag

SEQ ID NO: 181 - DNA, 94 bp, synthetic construct
  source 1..94: mol_type = other DNA; note = Forward sequence ORF 36
ttgcccccggtgtgccctgaaactccctaaggctacccggtaggtagataggatttcagagagaccctgggcaggatgacgacgataagtaggg

SEQ ID NO: 182 - DNA, 96 bp, synthetic construct
  source 1..96: mol_type = other DNA; note = Reverse sequence ORF 36
gattcagctgccatgtggacgcccagggtctctctgaaatcctatctacctaccgggtagccttagggagttaaccaattaaccaattctgattag

SEQ ID NO: 183 - DNA, 94 bp, synthetic construct
  source 1..94: mol_type = other DNA; note = Forward sequence ORF 37
agaaagctactagagcgagactttttcaaccatggaggcctaggtagataggacccccacacccgcggacttaggatgacgacgataagtaggg

SEQ ID NO: 184 - DNA, 96 bp, synthetic construct
  source 1..96: mol_type = other DNA; note = Reverse sequence ORF 37
ccagatagtcttcagaaaacaagtccgcgggtgtgggggtcctatctacctaggcctccatggttgaaaaagaaccaattaaccaattctgattag

SEQ ID NO: 185 - DNA, 94 bp, synthetic construct
  source 1..94: mol_type = other DNA; note = Forward sequence ORF 38
gaagtgcgaaggacacctttccatatatcaaatgggattttaggtagataggctcctatctatctgcaaacgaggatgacgacgataagtaggg

SEQ ID NO: 186 - DNA, 96 bp, synthetic construct
  source 1..96: mol_type = other DNA; note = Reverse sequence ORF 38
cgtctacgggctgtgagggacgtttgcagatagataggagcctatctacctaaaatcccatttgatatatggaaccaattaaccaattctgattag

SEQ ID NO: 187 - DNA, 82 bp, synthetic construct
  source 1..82: mol_type = other DNA; note = Forward sequence ORF 39
atggaggaagagggatgggtttataatgccaatatatcaggtttctcggtctttttaactaggatgacgacgataagtaggg

SEQ ID NO: 188 - DNA, 84 bp, synthetic construct
  source 1..84: mol_type = other DNA; note = Reverse sequence ORF 39
ccgcagcgccgcctggcgaaagttaaaaagaccgagaaacctgatatattggcattataaaaccaattaaccaattctgattag

SEQ ID NO: 189 - DNA, 82 bp, synthetic construct
  source 1..82: mol_type = other DNA; note = Forward sequence ORF 40
gccccgggcagaagccagaggtagtcgactcattgactcaagcggagagggggtggtgcgaggatgacgacgataagtaggg

SEQ ID NO: 190 - DNA, 84 bp, synthetic construct
  source 1..84: mol_type = other DNA; note = Reverse sequence ORF 40
aaacccgtcaactgccaactcgcaccaccccctctccgcttgagtcaatgagtcgactacaaccaattaaccaattctgattag

SEQ ID NO: 191 - DNA, 94 bp, synthetic construct
  source 1..94: mol_type = other DNA; note = Forward sequence ORF 41
agaattcaaggatctcaaaagggcctgccagatggccgggtaggtagataggtttactctaaagggggggacaggatgacgacgataagtaggg

SEQ ID NO: 192 - DNA, 96 bp, synthetic construct
  source 1..96: mol_type = other DNA; note = Reverse sequence ORF 41
agaatacaagatcccccgaagtccccccctttagagtaaacctatctacctacccggccatctggcaggcccaaccaattaaccaattctgattag

SEQ ID NO: 193 - DNA, 94 bp, synthetic construct
  source 1..94: mol_type = other DNA; note = Forward sequence ORF 42
aaatactgtctagttacaccacccttcgagaatgtccctgtaggtagatagggaaagggccctggcgagactaggatgacgacgataagtaggg

SEQ ID NO: 194 - DNA, 96 bp, synthetic construct
  source 1..96: mol_type = other DNA; note = Reverse sequence ORF 42
tactcattggcactccagtcagtctcgccagggccctttccctatctacctacagggacattctcgaagggtaaccaattaaccaattctgattag

SEQ ID NO: 195 - DNA, 94 bp, synthetic construct
  source 1..94: mol_type = other DNA; note = Forward sequence ORF 43
cactacgctcctgactttggcatccgatgtcatgttgaggtaggtagataggatgaacccggggctgggctcaggatgacgacgataagtaggg

SEQ ID NO: 196 - DNA, 96 bp, synthetic construct
  source 1..96: mol_type = other DNA; note = Reverse sequence ORF 43
aagggtgcactgatatggacgagcccagccccgggttcatcctatctacctacctcaacatgacatcggatgaaccaattaaccaattctgattag

SEQ ID NO: 197 - DNA, 94 bp, synthetic construct
  source 1..94: mol_type = other DNA; note = Forward sequence ORF 44
atacttgccttgcaggatctcaaagagggagatggacagctaggtagataggtcggaagggtgcactgatataggatgacgacgataagtaggg

SEQ ID NO: 198 - DNA, 96 bp, synthetic construct
  source 1..96: mol_type = other DNA; note = Reverse sequence ORF 44
acccggggctgggctcgtccatatcagtgcacccttccgacctatctacctagctgtccatctccctctttgaaccaattaaccaattctgattag

SEQ ID NO: 199 - DNA, 82 bp, synthetic construct
  source 1..82: mol_type = other DNA; note = Forward sequence ORF 45
acacctataatggtctgtattgacaccattcttttatttaggccttgtacggggttgaccaggatgacgacgataagtaggg

SEQ ID NO: 200 - DNA, 84 bp, synthetic construct
  source 1..84: mol_type = other DNA; note = Reverse sequence ORF 45
tgtaaatttccgcccctagcggtcaaccccgtacaaggcctaaataaaagaatggtgtcaaaccaattaaccaattctgattag

SEQ ID NO: 201 - DNA, 82 bp, synthetic construct
  source 1..82: mol_type = other DNA; note = Forward sequence ORF 46
gctaggggcggaaatttacaaagcacacgagttattgcctgttgaacttattttccctttaggatgacgacgataagtaggg

SEQ ID NO: 202 - DNA, 84 bp, synthetic construct
  source 1..84: mol_type = other DNA; note = Reverse sequence ORF 46
cggagagcgcacgcaggtcaaaagggaaaataagttcaacaggcaataactcgtgtgcttaaccaattaaccaattctgattag

SEQ ID NO: 203 - DNA, 82 bp, synthetic construct
  source 1..82: mol_type = other DNA; note = Forward sequence ORF 47
cctaaagaccgtctgttgcaaccatgcgtccatgttgaacggggcaaatccgggtttcacaggatgacgacgataagtaggg

SEQ ID NO: 204 - DNA, 84 bp, synthetic construct
  source 1..84: mol_type = other DNA; note = Reverse sequence ORF 47
ccgaaccaggcaacacaagggtgaaacccggatttgccccgttcaacatggacgcatggtaaccaattaaccaattctgattag

SEQ ID NO: 205 - DNA, 82 bp, synthetic construct
  source 1..82: mol_type = other DNA; note = Forward sequence ORF 48
ggaagacgatgggggaaatgtggcattacctgacacggttgttcagtcacatgtacgctaaggatgacgacgataagtaggg

SEQ ID NO: 206 - DNA, 84 bp, synthetic construct
  source 1..84: mol_type = other DNA; note = Reverse sequence ORF 48
ggggttgggtggggagaccctagcgtacatgtgactgaacaaccgtgtcaggtaatgccaaaccaattaaccaattctgattag

SEQ ID NO: 207 - DNA, 82 bp, synthetic construct
  source 1..82: mol_type = other DNA; note = Forward sequence ORF 49
acaaaaatggcgcaagatgacaaggtaaagatcgaccttttgtaaaaactatgacacgccaggatgacgacgataagtaggg

SEQ ID NO: 208 - DNA, 84 bp, synthetic construct
  source 1..84: mol_type = other DNA; note = Reverse sequence ORF 49
tcttaccctaaggagagagtggcgtgtcatagtttttacaaaaggtcgatctttaccttgaaccaattaaccaattctgattag

SEQ ID NO: 209 - DNA, 82 bp, synthetic construct
  source 1..82: mol_type = other DNA; note = Forward sequence ORF 50
atgtcattgtaaaaactatgacacgccactctctccttagagtgttcgcaagggcgtctgaggatgacgacgataagtaggg

SEQ ID NO: 210 - DNA, 84 bp, synthetic construct
  source 1..84: mol_type = other DNA; note = Reverse sequence ORF 50
ctgggaagttaacgcaggcacagacgcccttgcgaacactctaaggagagagtggcgtgtaaccaattaaccaattctgattag

SEQ ID NO: 211 - DNA, 82 bp, synthetic construct
  source 1..82: mol_type = other DNA; note = Forward sequence ORF K8
gtcgactataacctggcgtgtaaacgtgtaaccctgccaaacgggaaacaggtgtctatcaggatgacgacgataagtaggg

SEQ ID NO: 212 - DNA, 84 bp, synthetic construct
  source 1..84: mol_type = other DNA; note = Reverse sequence ORF K8
ttgagtaaccagccggccaagatagacacctgtttcccgtttggcagggttacacgtttaaaccaattaaccaattctgattag

SEQ ID NO: 213 - DNA, 82 bp, synthetic construct
  source 1..82: mol_type = other DNA; note = Forward sequence ORF K8.1
ggaccgaagttaatcccttaatcctctgggattaataacctggtgctagtaaccgtgtgcaggatgacgacgataagtaggg

SEQ ID NO: 214 - DNA, 84 bp, synthetic construct
  source 1..84: mol_type = other DNA; note = Reverse sequence ORF K8.1
tgtagtggtggcagaaaatggcacacggttactagcaccaggttattaatcccagaggataaccaattaaccaattctgattag

SEQ ID NO: 215 - DNA, 82 bp, synthetic construct
  source 1..82: mol_type = other DNA; note = Forward sequence ORF 52
gtttgggggttgggttgtggcgtggtggctggtccgcggtgtcaggtacgcgtagatgtaaggatgacgacgataagtaggg

SEQ ID NO: 216 - DNA, 84 bp, synthetic construct
  source 1..84: mol_type = other DNA; note = Reverse sequence ORF 52
ttgtgagcatcaccaacacgtacatctacgcgtacctgacaccgcggaccagccaccacgaaccaattaaccaattctgattag

SEQ ID NO: 217 - DNA, 82 bp, synthetic construct
  source 1..82: mol_type = other DNA; note = Forward sequence ORF 53
tagctttcgtcagcgcttgtgcgagtaatcacatgccagttatgaacaaccgccgaggctaggatgacgacgataagtaggg

SEQ ID NO: 218 - DNA, 84 bp, synthetic construct
  source 1..84: mol_type = other DNA; note = Reverse sequence ORF 53
acgttggatagacggcttggagcctcggcggttgttcataactggcatgtgattactcgcaaccaattaaccaattctgattag

SEQ ID NO: 219 - DNA, 82 bp, synthetic construct
  source 1..82: mol_type = other DNA; note = Forward sequence ORF 54
ggccacaaataaagccagggccaccgtggacgctgtcattagccgccgccaaatgcggccaggatgacgacgataagtaggg

SEQ ID NO: 220 - DNA, 84 bp, synthetic construct
  source 1..84: mol_type = other DNA; note = Reverse sequence ORF 54
tcgaatcgccctaataaactggccgcatttggcggcggctaatgacagcgtccacggtggaaccaattaaccaattctgattag

SEQ ID NO: 221 - DNA, 94 bp, synthetic construct
  source 1..94: mol_type = other DNA; note = Forward sequence ORF 55
gcctcgcggcggtatgtcgtctccatggtacacctggacgtaggtagataggtgttgcggtataaaccttttaggatgacgacgataagtaggg

SEQ ID NO: 222 - DNA, 96 bp, synthetic construct
  source 1..96: mol_type = other DNA; note = Reverse sequence ORF 55
aagcgtggttgccgcgtccaaaaaggtttataccgcaacacctatctacctacgtccaggtgtaccatggagaaccaattaaccaattctgattag

SEQ ID NO: 223 - DNA, 94 bp, synthetic construct
  source 1..94: mol_type = other DNA; note = Forward sequence ORF 56
cacgtccaggtgtaccatggagacgacataccgccgcgagtaggtagatagggcgctgacagtaagggttataggatgacgacgataagtaggg

SEQ ID NO: 224 - DNA, 96 bp, synthetic construct
  source 1..96: mol_type = other DNA; note = Reverse sequence ORF 56
tgtcgccactcgtacaaaaaataacccttactgtcagcgccctatctacctactcgcggcggtatgtcgtctaaccaattaaccaattctgattag

SEQ ID NO: 225 - DNA, 82 bp, synthetic construct
  source 1..82: mol_type = other DNA; note = Forward sequence ORF 57
aatataagaaccaaaggacatggtacaagcaatgatagacggattgccaaaccccatggcaggatgacgacgataagtaggg

SEQ ID NO: 226 - DNA, 84 bp, synthetic construct
  source 1..84: mol_type = other DNA; note = Reverse sequence ORF 57
tggaatacgggagacactctgccatggggtttggcaatccgtctatcattgcttgtaccaaaccaattaaccaattctgattag

SEQ ID NO: 227 - DNA, 82 bp, synthetic construct
  source 1..82: mol_type = other DNA; note = Forward sequence ORF K9
ctccctcccataacaatacggtgtaggcattttgtattattgtcccgcaaccagactagcaggatgacgacgataagtaggg

SEQ ID NO: 228 - DNA, 84 bp, synthetic construct
  source 1..84: mol_type = other DNA; note = Reverse sequence ORF K9
cactggacattgcggcgcgagctagtctggttgcgggacaataatacaaaatgcctacacaaccaattaaccaattctgattag

SEQ ID NO: 229 - DNA, 82 bp, synthetic construct
  source 1..82: mol_type = other DNA; note = Forward sequence ORF K10
actacaagattacatccggttttataattcacatatatgaacctgaggtagatgcgccctaggatgacgacgataagtaggg

SEQ ID NO: 230 - DNA, 84 bp, synthetic construct
  source 1..84: mol_type = other DNA; note = Reverse sequence ORF K10
cgtgtggataccagtgaatgagggcgcatctacctcaggttcatatatgtgaattataaaaaccaattaaccaattctgattag

SEQ ID NO: 231 - DNA, 82 bp, synthetic construct
  source 1..82: mol_type = other DNA; note = Forward sequence ORF K10.5
ccacagcccgtcaaaccacagggaccctgttggctgactacaatgcacatgcagattcttaggatgacgacgataagtaggg

SEQ ID NO: 232 - DNA, 84 bp, synthetic construct
  source 1..84: mol_type = other DNA; note = Reverse sequence ORF K 10.5; note = Reverse sequence ORF K 10.5
tgacctcacactgcttgataaagaatctgcatgtgcattgtagtcagccaacagggtcccaaccaattaaccaattctgattag

SEQ ID NO: 233 - DNA, 94 bp, synthetic construct
  source 1..94: mol_type = other DNA; note = Forward sequence ORF K11
ggtgggggctcagggttttgtagggagggatatgcacagttaggtagataggttgttttttgaagagccagaaggatgacgacgataagtaggg

SEQ ID NO: 234 - DNA, 96 bp, synthetic construct
  source 1..96: mol_type = other DNA; note = Reverse sequence ORF K11
atgacccaaaccccgacggttctggctcttcaaaaaacaacctatctacctaactgtgcatatccctccctaaaccaattaaccaattctgattag

SEQ ID NO: 235 - DNA, 82 bp, synthetic construct
  source 1..82: mol_type = other DNA; note = Forward sequence ORF 58
tcatggtcaacaaaccaagaaaaacacatgtattattcaaggtgtcaaatcagggggttaaggatgacgacgataagtaggg

SEQ ID NO: 236 - DNA, 84 bp, synthetic construct
  source 1..84: mol_type = other DNA; note = Reverse sequence ORF 58
aaggtgcccaaaaccacatttaaccccctgatttgacaccttgaataatacatgtgttttaaccaattaaccaattctgattag

SEQ ID NO: 237 - DNA, 82 bp, synthetic construct
  source 1..82: mol_type = other DNA; note = Forward sequence ORF 59
gagcgacagagcgcgctcactgtccaggcggcacatggtggattgcggccgtagacgcacaggatgacgacgataagtaggg

SEQ ID NO: 238 - DNA, 84 bp, synthetic construct
  source 1..84: mol_type = other DNA; note = Reverse sequence ORF 59
agctttcctgtgatttctctgtgcgtctacggccgcaatccaccatgtgccgcctggacaaaccaattaaccaattctgattag

SEQ ID NO: 239 - DNA, 82 bp, synthetic construct
  source 1..82: mol_type = other DNA; note = Forward sequence ORF 60
ttaggggaggtggaagtgtgcgacatggacaggttaaccttggcctcacccggcttgcagaggatgacgacgataagtaggg

SEQ ID NO: 240 - DNA, 84 bp, synthetic construct
  source 1..84: mol_type = other DNA; note = Reverse sequence ORF 60
gtctgtcagtaggtaggtctctgcaagccgggtgaggccaaggttaacctgtccatgtcgaaccaattaaccaattctgattag

SEQ ID NO: 241 - DNA, 82 bp, synthetic construct
  source 1..82: mol_type = other DNA; note = Forward sequence ORF 61
aactgaatccattggcctcacccggcttgcagagacctacgaccttacagaaacacagtcaggatgacgacgataagtaggg

SEQ ID NO: 242 - DNA, 84 bp, synthetic construct
  source 1..84: mol_type = other DNA; note = Reverse sequence ORF 61
gaggccgcgtgtggcccctggactgtgtttctgtaaggtcgtaggtctctgcaagccgggaaccaattaaccaattctgattag

SEQ ID NO: 243 - DNA, 82 bp, synthetic construct
  source 1..82: mol_type = other DNA; note = Forward sequence ORF 62
tcccaagtgaacctgacaaaatgtccggacagacatgaccatccacgccggcaatggacgaggatgacgacgataagtaggg

SEQ ID NO: 244 - DNA, 84 bp, synthetic construct
  source 1..84: mol_type = other DNA; note = Reverse sequence ORF 62
cttttcaagagcgtctgtgccgtccattgccggcgtggatggtcatgtctgtccggacataaccaattaaccaattctgattag

SEQ ID NO: 245 - DNA, 82 bp, synthetic construct
  source 1..82: mol_type = other DNA; note = Forward sequence ORF 63
agccgcattttcagcctgcaccttcatatccacgccggcaaggccatggcagcccagcctaggatgacgacgataagtaggg

SEQ ID NO: 246 - DNA, 84 bp, synthetic construct
  source 1..84: mol_type = other DNA; note = Reverse sequence ORF 63
gccattccctccatgtacagaggctgggctgccatggccttgccggcgtggatatgaaggaaccaattaaccaattctgattag

SEQ ID NO: 247 - DNA, 82 bp, synthetic construct
  source 1..82: mol_type = other DNA; note = Forward sequence ORF 64
cgctggcaggcctccggaaactgtttgtcgaatagaggccctccacggttgtccaatcgtaggatgacgacgataagtaggg

SEQ ID NO: 248 - DNA, 84 bp, synthetic construct
  source 1..84: mol_type = other DNA; note = Reverse sequence ORF 64
ctggcaaaaagaaataggcaacgattggacaaccgtggagggcctctattcgacaaacagaaccaattaaccaattctgattag

SEQ ID NO: 249 - DNA, 82 bp, synthetic construct
  source 1..82: mol_type = other DNA; note = Forward sequence ORF 65
agaagtggtacttgtgactccacggttgtccaatcgttgccttccacacaggcgggcgaaaggatgacgacgataagtaggg

SEQ ID NO: 250 - DNA, 84 bp, synthetic construct
  source 1..84: mol_type = other DNA; note = Reverse sequence ORF 65
agtgttcctcctgaggctatttcgcccgcctgtgtggaaggcaacgattggacaaccgtgaaccaattaaccaattctgattag

SEQ ID NO: 251 - DNA, 94 bp, synthetic construct
  source 1..94: mol_type = other DNA; note = Forward sequence ORF 66
taggccgtgcggtcgcgctggtgagaaggtcatggccctgtaggtagatagggatcagcgctgggatcgcttaggatgacgacgataagtaggg

SEQ ID NO: 252 - DNA, 96 bp, synthetic construct
  source 1..96: mol_type = other DNA; note = Reverse sequence ORF 66
aaccaaaccaagacacaagaaagcgatcccagcgctgatccctatctacctacagggccatgaccttctcacaaccaattaaccaattctgattag

SEQ ID NO: 253 - DNA, 94 bp, synthetic construct
  source 1..94: mol_type = other DNA; note = Forward sequence ORF 67
ggggccttgccagccccaccccgctgtcgccatgagtgtctaggtagatagggttggtaagcgtgtagtggaaggatgacgacgataagtaggg

SEQ ID NO: 254 - DNA, 96 bp, synthetic construct
  source 1..96: mol_type = other DNA; note = Reverse sequence ORF 67
ataccacccgacacagttcgtccactacacgcttaccaaccctatctacctagacactcatggcgacagcggaaccaattaaccaattctgattag

SEQ ID NO: 255 - DNA, 94 bp, synthetic construct
  source 1..94: mol_type = other DNA; note = Forward sequence ORF 67.5
gtctgaccagcttctgcctcgtgacatgcaaattttattttaggtagataggcccacgatctattgtagattaggatgacgacgataagtaggg

SEQ ID NO: 256 - DNA, 96 bp, synthetic construct
  source 1..96: mol_type = other DNA; note = Reverse sequence ORF 67.5
gacagtagttgatggcgttcaatctacaatagatcgtgggcctatctacctaaaataaaatttgcatgtcacaaccaattaaccaattctgattag

SEQ ID NO: 257 - DNA, 94 bp, synthetic construct
  source 1..94: mol_type = other DNA; note = Forward sequence ORF 68
tctacaatagatcgtgggaaataaaatttgcatgtcacgataggtagataggggcagaagctggtcagacgcaggatgacgacgataagtaggg

SEQ ID NO: 258 - DNA, 96 bp, synthetic construct
  source 1..96: mol_type = other DNA; note = Reverse sequence ORF 68
tggaacccaacatggagtacgcgtctgaccagcttctgcccctatctacctatcgtgacatgcaaattttataaccaattaaccaattctgattag

SEQ ID NO: 259 - DNA, 82 bp, synthetic construct
  source 1..82: mol_type = other DNA; note = Forward sequence ORF 69
acgcttgagctggtcccgggccttcgcaccccatccaccgcctcacatgtagcctgtcacaggatgacgacgataagtaggg

SEQ ID NO: 260 - DNA, 84 bp, synthetic construct
  source 1..84: mol_type = other DNA; note = Reverse sequence ORF 69
cagttgcaataggagctggggtgacaggctacatgtgaggcggtggatggggtgcgaaggaaccaattaaccaattctgattag

SEQ ID NO: 261 - DNA, 82 bp, synthetic construct
  source 1..82: mol_type = other DNA; note = Forward sequence ORF K12
attttattttactgacactctttgggagggcacgctagctgcattgggattggagtgaggaggatgacgacgataagtaggg

SEQ ID NO: 262 - DNA, 84 bp, synthetic construct
  source 1..84: mol_type = other DNA; note = Reverse sequence ORF K12
aacctggtgccctcctccctcctcactccaatcccaatgcagctagcgtgccctcccaaaaaccaattaaccaattctgattag

SEQ ID NO: 263 - DNA, 82 bp, synthetic construct
  source 1..82: mol_type = other DNA; note = Forward sequence ORF K13
catacattctacggaccaaaaattagcaacagcttgttatggtgccggcttgtatatgtgaggatgacgacgataagtaggg

SEQ ID NO: 264 - DNA, 84 bp, synthetic construct
  source 1..84: mol_type = other DNA; note = Reverse sequence ORF K13
tttttccacatcggtgccttcacatatacaagccggcaccataacaagctgttgctaattaaccaattaaccaattctgattag

SEQ ID NO: 265 - DNA, 82 bp, synthetic construct
  source 1..82: mol_type = other DNA; note = forward sequence ORF 72
aaggaaaatttatttttccgccctaaacaaaatcacaagcatagagtggcgagcgtatgtaggatgacgacgataagtaggg

SEQ ID NO: 266 - DNA, 84 bp, synthetic construct
  source 1..84: mol_type = other DNA; note = Reverse sequence ORF 72
ccaggctctagaggtaggccacatacgctcgccactctatgcttgtgattttgtttagggaaccaattaaccaattctgattag

SEQ ID NO: 267 - DNA, 82 bp, synthetic construct
  source 1..82: mol_type = other DNA; note = Forward sequence ORF 73
ggtggcttctagggaggaaaaagggggagaggtgtggcttcctcgggaaatctggtctgaaggatgacgacgataagtaggg

SEQ ID NO: 268 - DNA, 84 bp, synthetic construct
  source 1..84: mol_type = other DNA; note = Reverse sequence ORF 73
ccataattttactttggttgtcagaccagatttcccgaggaagccacacctctcccccttaaccaattaaccaattctgattag

SEQ ID NO: 269 - DNA, 82 bp, synthetic construct
  source 1..82: mol_type = other DNA; note = Forward sequence ORF K14
tgctcccccgtggacgacgccgagtgcctctcgggggtccctagatggacaccccgtgaaaggatgacgacgataagtaggg

SEQ ID NO: 270 - DNA, 84 bp, synthetic construct
  source 1..84: mol_type = other DNA; note = Reverse sequence ORF K14
ggggtgggtaagcacgacggttcacggggtgtccatctagggacccccgagaggcactcgaaccaattaaccaattctgattag

SEQ ID NO: 271 - DNA, 82 bp, synthetic construct
  source 1..82: mol_type = other DNA; note = Forward sequence ORF 74
cgtggctaaacaacacctatactacttgttattgtaggcccccgcggatgtctacgtgccaggatgacgacgataagtaggg

SEQ ID NO: 272 - DNA, 84 bp, synthetic construct
  source 1..84: mol_type = other DNA; note = Reverse sequence ORF 74
agattaaattaagggggaagggcacgtagacatccgcgggggcctacaataacaagtagtaaccaattaaccaattctgattag

SEQ ID NO: 273 - DNA, 82 bp, synthetic construct
  source 1..82: mol_type = other DNA; note = Forward sequence ORF 75
ttatgcgattaaatgaggggtctgatcccaaaagcaatgtgcctagagggtgccccgcccaggatgacgacgataagtaggg

SEQ ID NO: 274 - DNA, 84 bp, synthetic construct
  source 1..84: mol_type = other DNA; note = Reverse sequence ORF 75
actacagagggtgtccccgggggcggggcaccctctaggcacattgcttttgggatcagaaaccaattaaccaattctgattag

SEQ ID NO: 275 - DNA, 82 bp, synthetic construct
  source 1..82: mol_type = other DNA; note = Forward sequence ORF K15
acaacaactctattgtaagccctgtggatacctagtcaaaccctccacgaccacagacttaggatgacgacgataagtaggg

SEQ ID NO: 276 - DNA, 84 bp, synthetic construct
  source 1..84: mol_type = other DNA; note = Reverse sequence ORF K15
aaaaaggtatcgatgtcaaaaagtctgtggtcgtggagggtttgactaggtatccacaggaaccaattaaccaattctgattag

SEQ ID NO: 277 - DNA, 30 bp, synthetic construct
  source 1..30: mol_type = other DNA; note = Forward sequence ORF r59 of Step 1
gagtccaagcttatgtcgcacacttccacc

SEQ ID NO: 278 - DNA, 30 bp, synthetic construct
  source 1..30: mol_type = other DNA; note = Reverse sequence ORF r59 of Step 1
gagtccgaattcaacgagtacagggccttg

SEQ ID NO: 279 - DNA, 84 bp, synthetic construct
  source 1..84: mol_type = other DNA; note = Forward sequence ORF r59 of Step 2
gagtccacgcgtgagctattcggtgcgaatgtactcgacgctggcatagccttttatcgaaaaggatgacgacgataagtaggg

SEQ ID NO: 280 - DNA, 36 bp, synthetic construct
  source 1..36: mol_type = other DNA; note = Reverse sequence ORF r59 of Step 2
gagtccacgcgtaaccaattaaccaattctgattag

SEQ ID NO: 281 - DNA, 19 bp, synthetic construct
  source 1..19: mol_type = other DNA; note = Forward sequence ORF r59 of Step 3
tcgtctccagaacacccag

SEQ ID NO: 282 - DNA, 23 bp, synthetic construct
  source 1..23: mol_type = other DNA; note = Reverse sequence ORF r59 of Step 3
cagataactgaagagcgacagag

SEQ ID NO: 283 - DNA, 34 bp, synthetic construct
  source 1..34: mol_type = other DNA; note = Forward sequence ORF r62 of Step 1
gagtccgcatgcttccatcaacagctttgtctgg

SEQ ID NO: 284 - DNA, 32 bp, synthetic construct
  source 1..32: mol_type = other DNA; note = Reverse sequence ORF r62 of Step 1
gagtccgaattctgagagattgggcacacata

SEQ ID NO: 285 - DNA, 84 bp, synthetic construct
  source 1..84: mol_type = other DNA; note = Forward sequence ORF r62 of Step 2
gagtccccatggtccagcgccgggaaccggcaggcctaaacgttactatttatgcctcgttgaggatgacgacgataagtaggg

SEQ ID NO: 286 - DNA, 36 bp, synthetic construct
  source 1..36: mol_type = other DNA; note = Reverse sequence ORF r62 of Step 2
gagtccccatggaaccaattaaccaattctgattag

SEQ ID NO: 287 - DNA, 18 bp, synthetic construct
  source 1..18: mol_type = other DNA; note = Forward sequence ORF r62 of Step 3
ttgtctggtgaaggctcc

SEQ ID NO: 288 - DNA, 19 bp, synthetic construct
  source 1..19: mol_type = other DNA; note = Reverse sequence ORF r62 of Step 3
gggacagctcccaagtgaa

(19) United States
(12) Patent Application Publication
(10) Pub. No.: US 2024/0248080 A1
(43) Pub. Date: Jul. 25, 2024

(54) Kaposi sarcoma associated herpesvirus gene function

(71) Applicant: The Regents of the University of California, Oakland, CA (US)

(72) Inventors: Fenyong Liu, Berkeley, CA (US); Adam Smith, Berkeley, CA (US); Marco Paliza-Carre, Berkeley, CA (US); Rachael Burchfield, Berkeley, CA (US)

(73) Assignee: The Regents of the University of California, Oakland, CA (US)

(21) Appl. No.: 18/629,922

(22) Filed: Apr. 8, 2024

Related U.S. Application Data
(63) Continuation of application No. PCT/US22/47515, filed on Oct. 24, 2022.
(60) Provisional application No. 63/271,704, filed on Oct. 25, 2021.

(51) Int. Cl. G01N 33/50; A61K 39/00; A61K 39/245; C07K 14/005; C12N 7/00

(52) U.S. Cl. CPC G01N 33/5091; A61K 39/245; C07K 14/005; C12N 7/00; A61K 2039/5254; A61K 2039/80; C12N 2710/16022; C12N 2710/16052; G01N 2333/03

(57) ABSTRACT

Kaposi's sarcoma-associated herpesvirus (KSHV) is an opportunistic pathogen causing Kaposi's sarcoma. It is capable of establishing latent infection, which can be reactivated to engage lytic infection for progeny production. KSHV contains a ˜165 kilobase DNA genome predicted to encode at least 90 open reading frames (ORFs). In this report, we generated 91 KSHV mutants, each characterized by the disruption of a single viral ORF. The growth of these mutants in cultured cells was examined to systematically investigate the necessity of each ORF for viral latency, reactivation, and lytic replication. Salient aspects are (a) 44 ORFs are essential for viral lytic replication in cultured cells and 47 are nonessential; (b) KSHV reactivation can be positively or negatively regulated by specific viral ORFs; and (c) ORFs identified to regulate viral reactivation encode functions modulating both innate and adaptive immune responses. The intersection of viral immunomodulatory genes controlling reactivation suggests that KSHV engages in a concerted effort to communicate and respond to the host immune system for reactivation and replication using a viral sensory network. Our results imply a novel mechanism in which reactivation of KSHV is actively controlled by the virus in response to its surrounding environment, leading to the opportunistic nature of viral diseases that are strongly correlated to the host's immune status and conditions.

### CROSS-REFERENCES TO RELATED APPLICATIONS

This application is a continuation of PCT/US22/47515, filed Oct. 24, 2022, which claims priority to U.S. Provisional Application No. 63/271,704 filed Oct. 25, 2021, the disclosures of which are hereby incorporated by reference in its entirety for all purposes.

### REFERENCE TO A SEQUENCE LISTING

A Sequence Listing in XML format is incorporated by reference into the specification. The name of the XML file containing the Sequence Listing is SeqList.xml. The XML file is 342,331 bytes and was created and submitted electronically via EFS-Web on Apr. 8, 2024.

### INTRODUCTION

Kaposi Sarcoma Associated Herpesvirus (KSHV) is a medically important virus with infection found globally. It is the causative agent for Kaposi sarcoma (KS), one of the AIDS defining complications. Furthermore, KSHV causes two other human diseases, primary effusion lymphoma and multicentric Castleman's disease. This virus infects many cell types including endothelial cells and B cells. Currently there are no drugs available to eliminate KSHV latent infection. Once infected with KSHV, the individual is infected for life. No vaccines against KSHV infection are currently available. There are urgent needs for developing drugs and vaccines for treatment and prevention of KSHV infection and KSHV-associated diseases including KS.

### SUMMARY OF THE INVENTION

The invention provides Kaposi sarcoma associated herpesvirus (KSHV) relevant methods and compositions, including antivirals, vaccines, and vectors.

The invention provides novel antiviral targets and gene function methods resulting from our comprehensive analysis of KSHV, and KSHV opportunistic factors with dual functions of regulating both the immune environment/responses and viral reactivation/replication. These viral factors that serve dual roles represent a novel strategy of achieving pathogen opportunistic pathogenesis, and have implications for the entire field of infectious diseases.

The disclosed, systematic analysis of the KSHV genome represents the most extensive global characterization of this virus. The results from this study, such as the identification of 44 viral ORFs essential for viral replication and the characterization of 47 growth-dispensable viral genes, enable new strategies and novel approaches for treatment and prevention of KSHV as well as other herpesviruses.

We disclose that KSHV encodes genes that have dual functions of regulating viral reactivation/replication and modulating host immune environment/response. We identified viral mutants with inactivation in genes that exhibit enhanced or reduced reactivation/lytic replication phenotypes as compared to the wild type virus. These inactivated ORFs with immunomodulatory functions encode factors that regulate viral reactivation and replication in connection with the host immune environment/status and responses. These ORFs are examples of virally encoded components that facilitate pathogen opportunistic activities and responses. In addition to KSHV, pathogen opportunistic responses may be a strategy employed by other infectious agents to enhance their long-term survivability within their respective host population.

The invention provides methods for developing drugs mimicking or activating opportunistic factors that inhibit viral reactivation/replication and enhance host immune responses may lead to effective therapies against infectious diseases. Similar antiviral effects can also be achieved by developing compounds that block or inactivate opportunistic factors that enhance viral reactivation/replication and suppress host immune responses. In vitro hyper-growth strains can be used for facile production of large quantity of subunit and attenuated live vaccines.

In aspects and embodiments the invention provides:
1. In one embodiment of the invention, a KSHV mutant comprising a deletion or mutation in one ORF, and libraries of such KSHV mutants, are provided. These mutant viruses enable further genetic alteration, e.g. in the deletion of a second ORF from a mutant, to add back a genetically engineered versions of the deleted ORF, and the like.
2. Open reading frames identified as essential for viral growth, and ORFs when inactivated lead to severe growth attenuated virus, can be targeted by anti-viral drugs designed to treat a KSHV infection in humans. Therapeutic agents that may be developed against these identified viral genes may include, but are not limited to, nucleic acid based compounds that target the mRNA transcribed from these essential regions, small molecule compounds designed to inhibit or bind to the protein molecules coded by these essential genes, or recombinant protein based molecules such as monoclonal antibodies which may bind to the protein products encoded by these essential genes.
3. Open reading frames identified herein, that when inactivated result in mutant viruses that are categorized as severe or moderate growth attenuated can be used to construct KSHV vaccines. The inactivation or deletion of the aforementioned genes results in attenuated viral growth in tissue culture ranging from 10-fold less than wild-type to severe growth defect compared to wild-type. These ORFs can be inactivated or deleted to create an attenuated or weakened virus which can then be used for vaccination against KSHV infection. Furthermore, ORFs identified as encoding cell tropism factors can also be deleted in vaccine constructs to prevent the vaccine strain from potentially causing disease in specific tissues. For example, ORFs encoding tropism factors for KSHV replication in human B cells can be inactivated or deleted from the vaccine construct to prevent the possibility that the vaccine may cause KSHV diseases associated with B cells.
4. Open reading frames identified herein that when inactivated result in viral mutants that exhibit no growth attenuation compared to the parental virus can be inactivated or deleted for construction of gene therapy vectors. Inactivation or deletion of these genes results in no significant deviation of viral growth from that of wild-type levels. This indicates that these regions can be inactivated or deleted from the viral genome without affecting viral growth in vitro. Inactivation and deletion of these genes provides more space in the viral genome to accommodate foreign genes being expressed in a gene therapy procedure. Identification of these “no growth attenuation” genes provides an advantage over other attenuated dispensable genes in that high-titers of the gene therapy vector can be attained due to the conservation of near to wild-type like growth characteristics in tissue culture.
5. A recombinant KSHV mutant containing several ORF deletions, as well as insertions of foreign genetic elements, can be used for an oncolytic viral therapy. Such mutations include the deletion of tropism factor ORFs that result in productive replication only in diseased cells. Secondary mutations can be made to further attenuate virulence in healthy cells, e.g. the deletion of ORF's essential for the maintenance of latency.

In aspects and embodiments the invention provides:
1. A Kaposi sarcoma associated herpesvirus (KSHV) mutant with inactivation or deletion of one or more of the 91 predicted open reading frames (91 mutant strains), as disclosed.

Methods of using the mutant viruses of claim 1 in applications such as research (e.g. for analyzing the molecular, cellular, and immunological response to mutant virus infections), and industry (e.g. as a “helper-virus” in the production of other viral vectors, and/or the generation of live-attenuated vaccines.
2. Methods and reagents (e.g. primers) for construction of the collection of the disclosed KSHV mutants, as disclosed.

Methods of mutagenesis having high fidelity (e.g. insert or remove a desired sequence with single nucleotide resolution), superior to other mutagenesis approaches like CRISPR.

Methods for reconstituting mutant viruses (e.g. using transfection, induction and tittering) comprising a tractable workflow.
3. Artificial constructs comprising one of the 44 KSHV essential genes, and use as antiviral targets, particularly the 27 new identified essential genes, as disclosed.

The identification of genetic sequences that are essential for viral reproduction, and their insertion into artificial constructs (e.g. protein expression plasmids).

Methods and reagents for high throughput, in-vitro drug screening assays to identify novel antivirals for KSHV, and other human herpesviruses, based hereon.

Development of other therapeutic approaches including monoclonal antibodies and nucleic acid therapies for KSHV infection.
4. Methods for construction of gene-inactivation and rescued mutants, and for tagging and introducing foreign genes into the KSHV genome, particularly for use in vector and vaccine development, as disclosed.

KSHV provides many useful features for vector development including its low seroprevalence in the developed world (which circumvents the pre-existing immunity problem encountered with adeno-associated virus (AAV) vectors), its ability to accommodate large transgene payloads (up to 50 kb in KSHV compared to 5 kb in current AAV approaches), and the absence of viral integration into the host genome.
5. Use of growth properties of viral mutants with inactivation of non-essential genes as disclosed.

Non-essential genes that impart severely attenuated growth, and thus their growth properties provide advantageous live-attenuated vaccine candidates.

Growth properties of non-attenuated mutants indicate genome regions that can be modified to contain foreign transgenes without affecting the growth properties of the virus in-vitro. These regions are useful in the development of KSHV-based vectors as their disruption/replacement will not affect viral growth during the manufacturing process. The growth properties of viral mutants under different conditions is also useful for identifying viral factors that regulate viral reactivation. These properties provision novel therapies for KSHV; for example, drugs targeting regulators of reactivation can be used to enhance reactivation and stimulate host-mediated immune clearance of latent virus infection, or, the repress viral reactivation to eliminate persistent infection.
6. Methods for screening mutants in different human cell lines as disclosed.

Screening results provide valuable assessments of the efficacy and safety of therapeutics targeting KSHV infected cells, as well as KSHV vaccines and KSHV based vectors.
7. Use of opportunistic factors of KSHV and all other animal viruses that have dual functions as both the modulators of immune environment/response and regulators of viral reactivation/replication, as disclosed.

Use and expression of these opportunistic viral immunomodulatory factors for KSHV therapy; for example, over-expressing an opportunistic factor that functions to suppress KSHV spontaneous reactivation, find use in the treatment of KSHV infection. 7. Use of opportunistic factors of KSHV and all other animal viruses that have dual functions as both the modulators of immune environment/response and regulators of viral reactivation/replication, as disclosed.

The invention encompasses all combinations of the particular embodiments recited herein, as if each combination had been laboriously recited.

### BRIEF DESCRIPTION OF THE DRAWINGS

FIGS. 1A-E. Characterization of KSHV mutants. (A-B) PCR products (A) and NheI digest (B) of DNAs purified from BAC16, deletion mutant ΔORF62, or rescued mutant rORF62. The red asterisks mark the digest band where ORF62 is expected to be found. (C) Microscopic images of parental and mutant BAC16-transfected iSLK cells under differential interference contrast (DIC) or for expression/staining of GFP, DAPI, and viral LANA. (D) Multi-step growth (MOI=0.1) of mutants and parental BAC16 in iSLK cells. (E) Lytic antigen ORF45 expression in mock-infected, BAC16-infected, and ΔORFK9-infected iSLK cells by flow cytometry. Experimental details can be found in Methods.

FIG. 2. Functional map of KSHV ORFs and their roles in viral growth in human cells. The genomic locations of KSHV ORFs are indicated by boxed arrows (accession: GQ994935.1). ORFs are colored coded based on the growth properties of their respective gene-inactivated mutants in iSLK cells (Table 1). The red asterisk marks the location where the BAC-backbone was inserted.

FIGS. 3A-E. Growth and lytic antigen expression of viral mutants under different conditions. (A-C). In multi-step growth conditions (A), iSLK cells were infected (MOI=0.1) in inducing conditions in the presence of doxycycline and sodium butyrate. Supernatants were harvested at 13 dpi. In induced reactivation conditions (B), iSLK cells were infected (MOI=1) and induced in the presence of doxycycline and sodium butyrate at 2 dpi, and supernatants were harvested at 5 dpi. In spontaneous reactivation conditions (C), iSLK cells were infected (MOI=1) and maintained in uninduced conditions and supernatants were harvested at 6 dpi. Mutant titers were normalized to BAC16 titers. (D-E). In (D), iSLK cells were infected (MOI=1), induced in the presence of doxycycline and sodium butyrate at 2 dpi and harvested at 4 dpi. In (E), iSLK cells were infected (MOI=1) and maintained in uninduced conditions and harvested at 6 dpi. The harvested cells were fixed, stained for lytic antigens, and analyzed by flow cytometry. The percentages of specific antigen expressing cells for mutants were normalized to those for BAC16. The values are the average of three independent experiments.

FIG. 4. Transfection and selection of BAC16 and ΔORF73 DNAs in iSLK cells. Cells were imaged using phase contract and fluorescence microscopy to visualize GFP after incubation with hygromycin B (1.2 mg/ml) for 6- and 62-days post transfection.

FIG. 5. KSHV growth in induced reactivation conditions. iSLK cells were either mock-infected (Mock) or infected with mutants (ΔORF38, ΔORF46, ΔORFK1, ΔORFK3, ΔORFK4, ΔORFK5) or parental BAC16 (BAC16) (MOI=1), and induced at 2 dpi. At 5 dpi the supernatants were harvested and used to infected 293T cells. Infected cells were fixed and analyzed by flow cytometry for GFP expression. PE (y-axis) was included as an autofluorescence control. The percentage of positive events is listed for each graph. The average of percentages in three independent experiments was used in the ratio calculations for the growth analysis in FIG. 3B.

FIG. 6. KSHV lytic antigen expression in induced reactivation conditions. iSLK cells were infected with mutants (ΔORFK3, ΔORFK4, and ΔORFK5) or parental BAC16 (BAC16) (MOI=1), induced in the presence of doxycycline and sodium butyrate at 2 dpi, harvested and stained at 4 dpi, and analyzed by flow cytometry for the expression of viral protein ORF45 (A, D, G, J), K8 (B, E, H, K), and K8.1 (C, F, I, L). PE (y-axis) was included as an autofluorescence control. The percentage of positive events is listed for each graph. The average of percentages in three independent experiments was used in the ratio calculations for the antigen expression ratios in FIG. 3D.

FIG. 7. KSHV growth in spontaneous reactivation conditions. iSLK cells were mock-infected or infected with mutants (ΔORF11AA, ΔORF61, ΔORF72, ΔORFK3, ΔORFK6, ΔORFK7, ΔORFK11) and parental BAC16 (BAC16) (MOI=1) and maintained in normal/uninduced conditions in the absence of doxycycline and sodium butyrate. Supernatants were harvested at 6 dpi and used to infected 293T cells. Infected cells were fixed and analyzed by flow cytometry for GFP expression. PE (y-axis) was included as an autofluorescence control. The percentage of positive events is listed for each graph. The average of percentages in three independent experiments was used in the ratio calculations for the growth analysis in FIG. 3C.

FIG. 8. KSHV lytic antigen expression in spontaneous reactivation conditions. iSLK cells were infected with mutants (ΔORF11AA, ΔORF49, ΔORF72, ΔORFK3, ΔORFK6, ΔORFK7, ΔORFK 11) and parental BAC16 (BAC16)(MOI=1) in uninduced conditions, and harvested and stained at 6 dpi, and analyzed by flow cytometry for the expression of viral protein ORF45 (A, D, G, J, M, P, S, V), K8 (B, E, H, K, N, Q, T, W), and K8.1 (C, F, I, L, O, R, U, X). PE (y-axis) was included as an autofluorescence control. The percentage of positive events is listed for each graph. The average of percentages in three independent experiments was used in the ratio calculations for the antigen expression ratios in FIG. 3E.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

Using a bacterial artificial chromosome (BAC) engineering and RED recombinase technology in conjunction with growth curve analysis in human cells in tissue culture, a viral mutant library with inactivation of each of 91 open reading frames spanning the entire KSHV genome was constructed. The BAC based ORF inactivation constructs were then transfected into human cells in tissue culture. Constructs with inactivation in 44 separate and distinct ORFs in the KSHV genome did not yield any viral progeny upon transfection into the human cells with induction, indicating that those regions of the genome are essential for viral growth and progeny production. This effort represents an exhaustive and complete mapping of the viral genome to identify all regions essential for viral growth and progeny production. These identified essential genes represent potential drug targets for anti KSHV therapeutic applications. In addition, the functional mapping of the genome has identified regions in the viral genome dispensable for viral growth and progeny production. All ORF inactivation constructs that yielded viral progeny upon transfection and induction were deemed dispensable for viral growth. Growth curve analyses were performed on the BAC derived mutant virus and the inactivated ORF categorized as either severe growth attenuation, moderate growth attenuation, no growth attenuation, or enhanced growth.

The identification of these non-essential genes distinguishes which genes can be inactivated or deleted to create an attenuated virus for use as a vaccine, or which genes can be inactivated or deleted to create a gene therapy vector so as to accommodate the delivery gene of interest without affecting viral propagation in vitro. Further growth kinetic characterization of the constructed mutants was carried out on different human cells such as human B cells and human microvascular endothelial cells and compared to the results from the human iSLK cell and 293T cell characterization. This comparative analysis identified ORF inactivation viruses that reactivated and replicated differentially, compared to the wild-type virus, in the cell types tested, indicating that these open reading frames encoded cell tropism important factors

### Examples: Global Functional Analysis of a Kaposi Sarcoma Associated Herpesvirus Genome

### Abstract

Kaposi's sarcoma-associated herpesvirus (KSHV) is an opportunistic pathogen causing Kaposi's sarcoma. It is capable of establishing latent infection, which can be reactivated to engage lytic infection for progeny production. KSHV contains a ˜165 kilobase DNA genome predicted to encode at least 90 open reading frames (ORFs). In this report, we generated 91 KSHV mutants, each characterized by the disruption of a single viral ORE. The growth of these mutants in cultured cells was examined to systematically investigate the necessity of each ORF for viral latency, reactivation, and lytic replication. Salient aspects are (a) 44 ORFs are essential for viral lytic replication in cultured cells and 47 are nonessential; (b) KSHV reactivation can be positively or negatively regulated by specific viral ORFs; and (c) ORFs identified to regulate viral reactivation encode functions modulating both innate and adaptive immune responses. The intersection of viral immunomodulatory genes controlling reactivation suggests that KSHV engages in a concerted effort to communicate and respond to the host immune system for reactivation and replication using a viral sensory network. Our results imply a novel mechanism in which reactivation of KSHV is actively controlled by the virus in response to its surrounding environment, leading to the opportunistic nature of viral diseases that are strongly correlated to the host's immune status and conditions.

### Introduction

Kaposi's sarcoma associated herpesvirus (KSHV) is an oncogenic gamma-herpesvirus which causes Kaposi's sarcoma, primary effusion lymphoma, and multicentric Castleman's disease¹. The other members of the human herpesvirus family include herpes simplex virus (HSV) 1 and 2, varicella zoster virus (VZV), Epstein Barr virus (EBV), cytomegalovirus (CMV), and human herpesviruses 6 and 7². A hallmark of herpesvirus infection is life-long persistence in a latent state with episodes of reactivation and lytic replication that correlate with the host's immune status and disease progression. During latency, herpesvirus genomes reside as episomes in the nucleus and only a few viral genes are expressed². Onset of reactivation from latency can occur in the presence of certain stimuli and is associated with changes in the host immune status. KSHV reactivation triggers viral lytic replication which proceeds via a highly regulated temporal cascade of gene expression, resulting in viral DNA replication and the assembly and release of infectious virions from the cell¹.

Reactivation and lytic replication of KSHV play important roles in the development of KSHV-associated disease as mechanisms for infection of naïve cells, through oncogenic effects of certain lytic proteins and paracrine signaling¹. However, the roles of individual viral genes in reactivation and lytic replication are not fully elucidated. Also, little is known about the processes and factors linking KSHV reactivation and changes in the host immune status.

Global studies assaying the essentiality of viral genes in several herpesviruses have been reported but were limited to studying lytic replication and not reactivation or latency³⁻⁷. KSHV consists of a ˜165 kb genome that has been predicted to encode at least 90 open reading frames (ORFs) including small and upstream ORFs, and numerous non-coding RNAs including miRNAs and circRNAs⁸⁻¹⁴. Only a handful of KSHV ORFs have been studied using gene inactivation mutants¹⁵⁻⁴².

In this report, we performed genome-wide mutational analysis and constructed 91 ORF-inactivating mutants using the KSHV BAC16 construct. BAC16 contains a KSHV genome cloned as a bacterial artificial chromosome (BAC)³⁸. Resembling KSHV infection in vivo, virus infection from the BAC16 construct in human iSLK cells typically leads to latency, and reactivation and lytic replication from this system can be induced⁴³. We studied ORF-inactivating mutants and investigated the roles of viral ORFs in KSHV latency, reactivation, and lytic replication. Notably, our study is the first global functional profiling of a KSHV genome.

### Results

### Generation of KSHV Mutants and Identification of ORFs Important for Latency

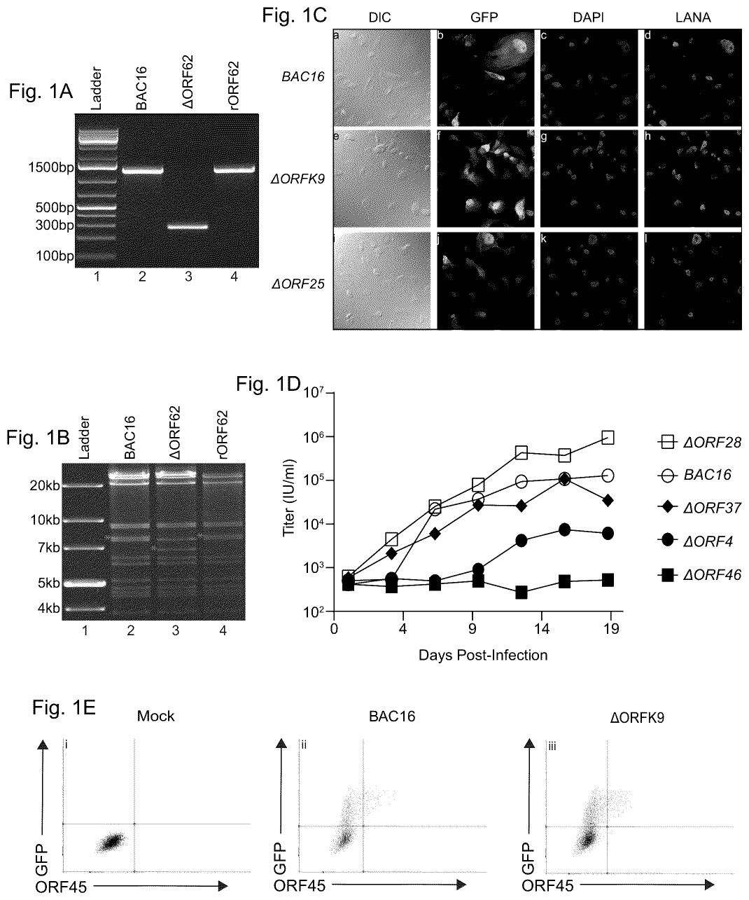

To generate mutant viruses, previous studies used a 2-step red-mediated recombination with BAC16 followed by transfection into iSLK cells, establishment of transfected cell populations, and induction of lytic replication¹⁵⁻⁴⁰. We used this approach to construct 91 BAC16 mutants. Each mutant has an inactivating mutation in a single ORF consisting of either a complete ORF deletion (nonoverlapping ORFs) or an insertion of a stop codon in each frame in the ORF 5′ region (overlapping ORFs). Mutant BAC16 DNAs were screened by PCR with primers (Table S1) designed to produce a unique and recognizable product (e.g. a ˜300 bp PCR product for ΔORF62) (FIG. 1A). Stop codon insertion was confirmed by sequencing. The overall genomic structures of the mutants were further examined using restriction digest profiling to assess if unexpected genomic rearrangements occurred (FIG. 1B).

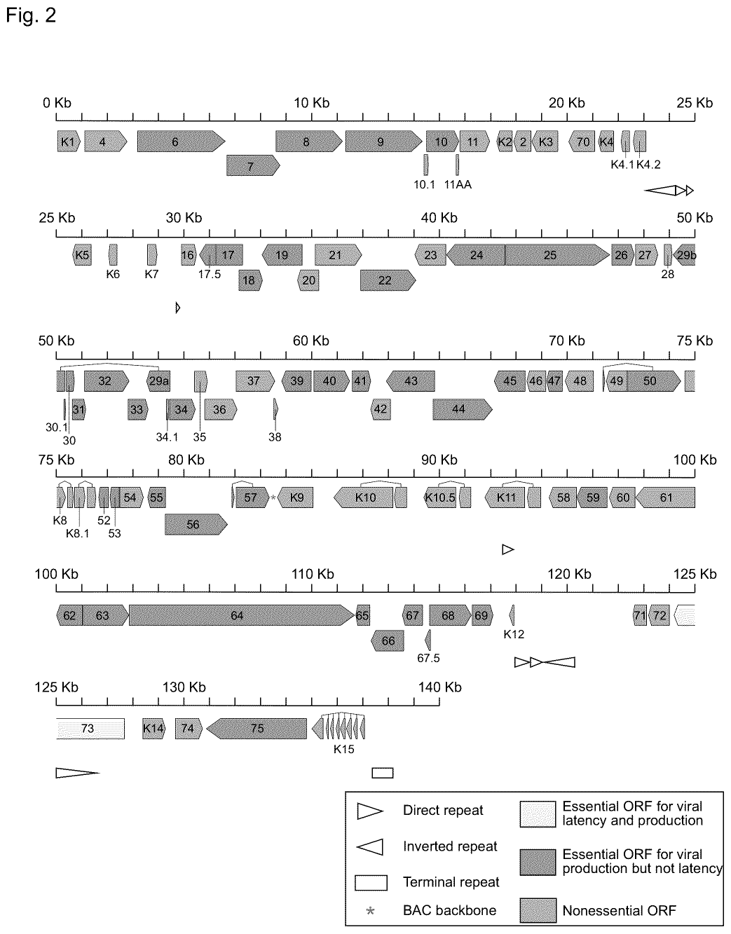

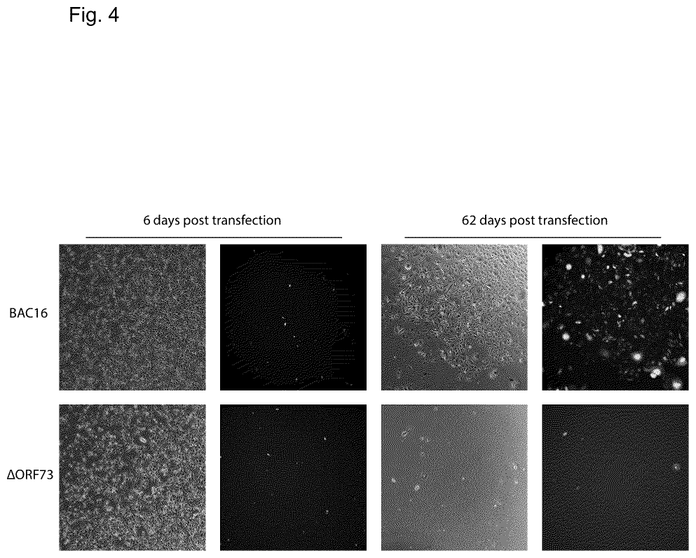

To reconstitute virus, iSLK cells were transfected with mutant or parental BAC16 DNAs and selected with hygromycin B. This produced populations of GFP+ cells as BAC16 contained a GFP expression cassette. To confirm KSHV infection, we also examined the expression of ORF73-encoded latency associated nuclear antigen (LANA) (FIG. 1C). Consistent with the essential role of ORF73 for viral latency^{17,44}, we could not generate cell populations harboring ΔORF73 even after repeated attempts and growing for more than 62 days (FIG. 4). In contrast, GFP+ and LANA+ cells were found with the remaining 90 mutants, indicating that these 90 ORFs are dispensable for establishment and maintenance of viral latency (Table 1, FIG. 1C, FIG. 2).

### Identification of ORFs Essential for Virus Production

Lytic replication was induced in transfected cell lines by doxycycline and sodium butyrate treatment and the supernatants were harvested 96 hours post-induction and titered on 293T cells (FIG. 1D-E). Forty-seven mutants produced infectious viral progeny, indicating that the mutated ORFs are not essential for KSHV replication in iSLK cells (Table 1). In contrast, 44 mutants did not yield viral progeny even after repeated attempts with independent transfections and extensive induction. To further confirm their no-growth phenotype, rescued BAC clones were derived from several mutants (e.g. ΔORF62) by restoring the mutations with the intact ORF sequence (FIG. 1A-B, Table S2). The rescued mutants (e.g. rORF62) produced progeny and grew as well as BAC16, confirming that the mutation inactivating the ORF causes the no-growth phenotype (Table 1, Table S3).

The majority of the 44 essential ORFs identified are conserved among herpesviruses with key roles in virus production, such as structural, enzymatic, and regulatory functions (FIG. 2, Table 1, Table S3). Strikingly, 10 conserved genes (ORFs 20, 23, 36, 37, 38, 42, 46, 54, 60, and 61) were nonessential for KSHV production². In contrast, ORFs 45, 50, 52, 73, and 75, which have no homologues in alpha and beta-herpesviruses, were essential (Table 1, Table S3). These 44 essential genes represent novel and ideal targets for antiviral drug development against KSHV infection.

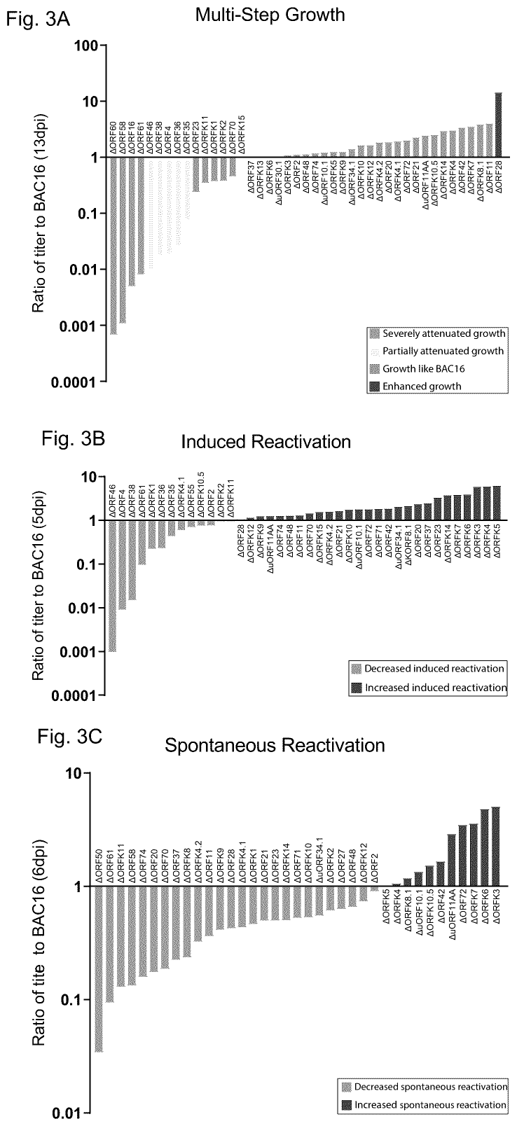

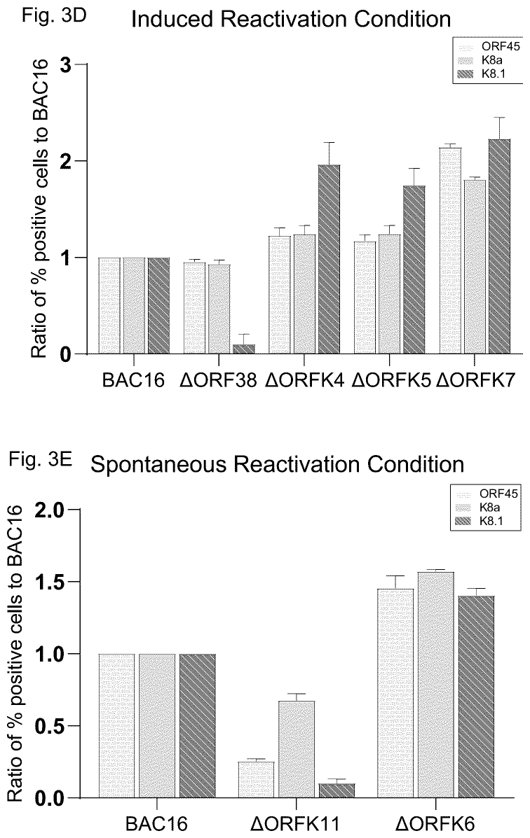

The growth of mutants with inactivation of nonessential ORFs was further analyzed under multi-step growth conditions for 19 days (FIG. 1D, FIG. 3A). Based on their peak titers, mutants could be categorized into four major groups: those for which virus production was severely-attenuated (at least 100-fold lower—9 mutants), partially-attenuated (10 to 100-fold lower—4 mutants), non-attenuated (within 10-fold—32 mutants), or enhanced (at least 10-fold higher—2 mutants) compared to parental BAC16 (FIG. 1D, FIG. 3A, Table 1). Notably, inactivation of conserved ORFs 20, 23, 37, and 42 showed no attenuation, while most mutants exhibiting no attenuation and enhanced growth had mutations at γ-herpesvirus or KSHV-specific genes (FIG. 3A, Table 1, Table S3).

### Identification of KSHV Genes Modulating Reactivation and Latency

To assay virus generated from reactivation and subsequent lytic replication, we infected iSLK cells, induced reactivation at 2 days post-infection (dpi), and harvested the supernatants at 5 dpi for titration. At 2 dpi prior to induction, we barely detected virus from the supernatant collected from BAC16-infected cells, suggesting establishment of viral latency and lack of reactivation. This conclusion is consistent with our observations that the percentage of parental BAC16-infected cells expressing ORF45 (an immediate early gene), K8 (an early gene), or K8.1 (a late gene) was 1.24%, 0.61%, and 0.33% respectively, suggesting that over 98% of BAC16-infected cells were not undergoing lytic replication (Table S4).

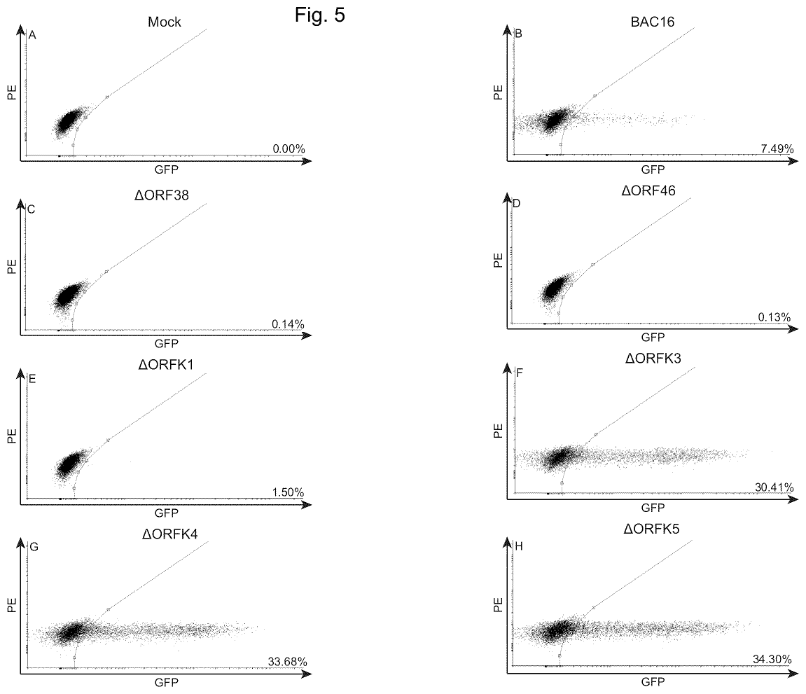

We expected to observe changes in virus production due to deficiencies or enhancements in reactivation or subsequent lytic replication. Most mutants generated a titer within 10-fold of parental BAC16 (FIG. 3B). While ΔORF38 and ΔORF46 generated titers more than 50-fold less than BAC16 (FIG. 3B, FIGS. 5-8), they were also attenuated in the lytic multi-step growth analysis, indicating that these ORFs likely do not play a role specific to reactivation (Table 1, FIG. 3A). However, ΔORFK3 and ΔORFK5, which exhibited little change in the multi-step growth analysis (FIG. 3A, Table 1), showed enhanced virus production (FIG. 3B, FIG. 5), implying that ORFK3 and ORFK5 may specifically suppress reactivation but not viral lytic replication.

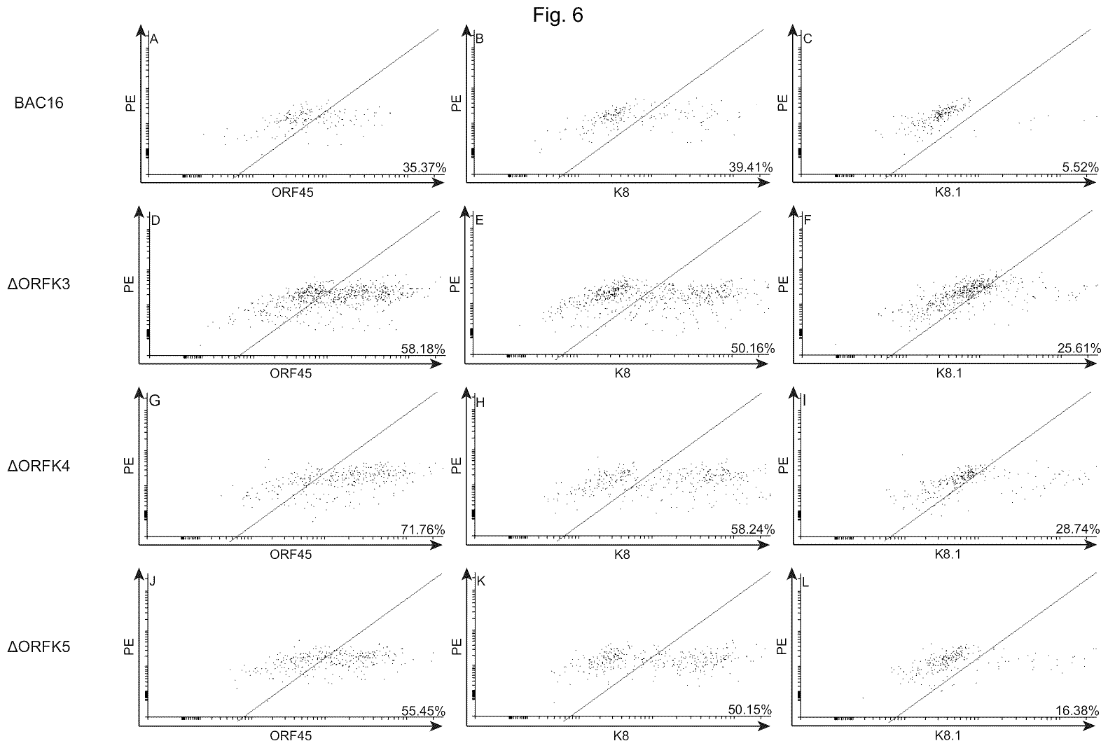

Increased virus production possibly results from enhanced lytic antigen expression. To test this hypothesis, we measured the expression of viral ORFs 45, K8 and K8.1 proteins under the same conditions. Mutants ΔORFK3 and ΔORFK5 showed an increased percentage of lytic antigen-expressing cells relative to parental BAC16 (FIG. 3D, FIG. 6), indicating that inactivating these genes, which are immunomodulatory factors^{45,46} enhanced reactivation at the gene expression level.

Next, we took advantage of our unique system to identify viral ORFs regulating latency and spontaneous reactivation by measuring virus production in the absence of lytic induction. At 6 dpi, the percentage of parental BAC16-infected cells expressing ORF45, K8, or K8.1 was 0.31%, 0.25%, 0.09% respectively, suggesting establishment of latency and lack of reactivation and lytic replication in over 99.5% of infected cells (Table S4). Thus, any change in virus production probably results from alteration of latency and spontaneous reactivation due to the inactivation of the ORF in the mutant.

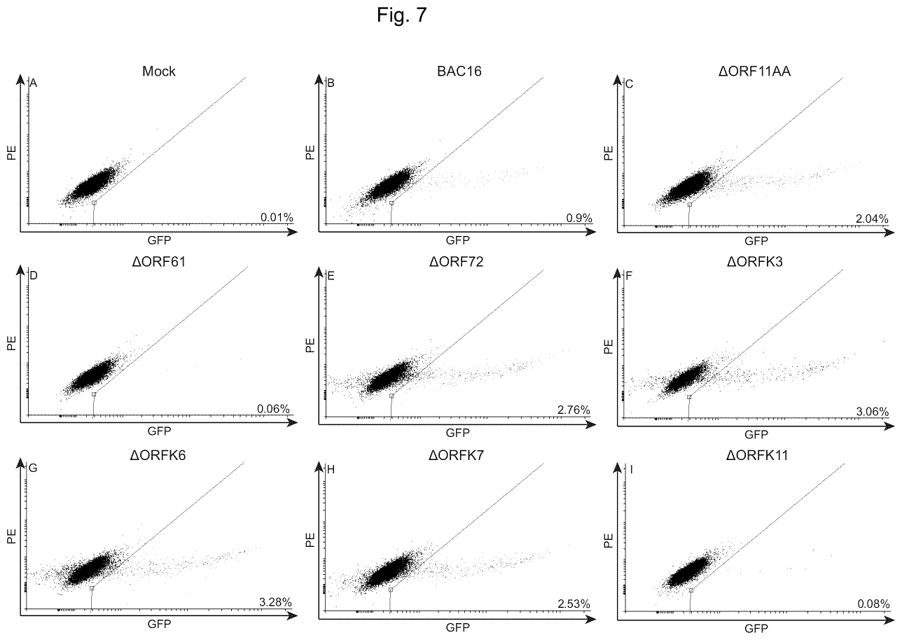

Consistent with previous observations that ORF50 is necessary and sufficient for reactivation 4748, ΔORF50 showed a 30-fold decrease in virus production relative to parental BAC16 (FIG. 3C). This confirmed the validity of the experimental system to study spontaneous reactivation. Although many mutants showed no attenuation in virus production, a few (e.g. ΔORF61, and ΔORFK11) exhibited a decrease of approximately 10-fold or more compared to parental BAC16 (FIG. 3C, FIG. 7). ΔORF61 was attenuated under multi-step growth and “induced” reactivation conditions (Table 1, FIGS. 3A and B). However, ΔORFK11 showed little attenuation in these assays, suggesting that the reduced progeny production under uninduced conditions is due to the specific role of ORFK11 in enhancing spontaneous reactivation and inhibiting latency (FIG. 3A, FIG. 3B, FIG. 7).

Several mutants (e.g. ΔORF11AA, ΔORF72, ΔORFK3, ΔORFK6, and ΔORFK7) achieved enhanced virus production (FIG. 3C, FIG. 7). ΔORFK7 showed enhanced growth under the multi-step growth conditions and during “induced” reactivation while ΔORFK3 and ΔORFK6 exhibited increased growth only during “induced” reactivation (FIG. 3A-C). In contrast, ΔORF11AA and ΔORF72 showed little enhanced growth under these two conditions, suggesting that ORF11AA and ORF72 specifically repress spontaneous reactivation and promote latency. Our results further imply that ORFK7 represses spontaneous reactivation, and in addition, possibly suppresses viral lytic replication steps.

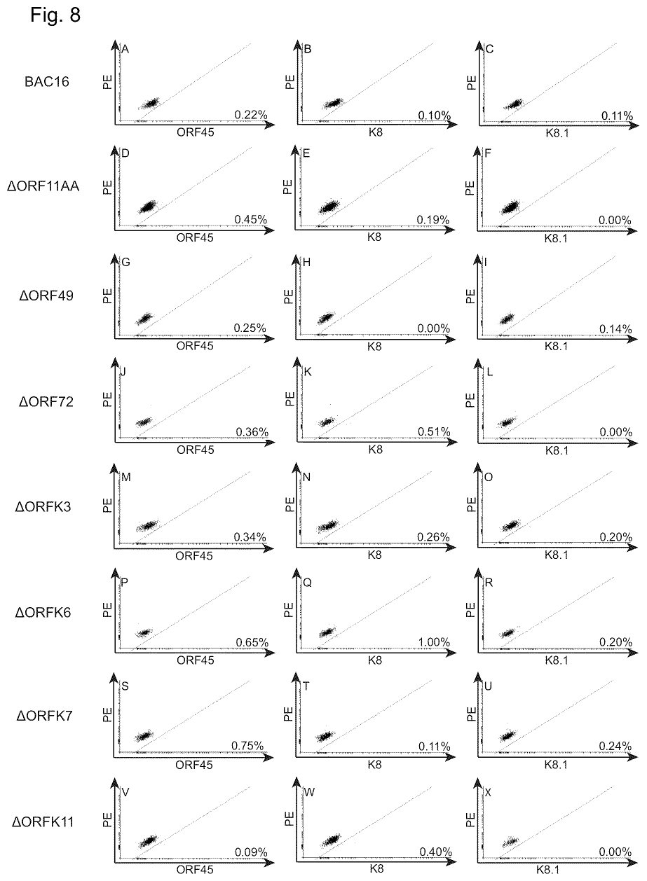

We then measured the percentage of infected cells expressing ORF45, K8, or K8.1 under these conditions to understand the correlation between viral lytic gene expression and altered levels of reactivation and virus production (FIG. 8). Interestingly, disruption of K6, an immunomodulatory factor encoding a viral chemokine homologue^{49,50}, increased lytic gene expression and virus production (FIG. 3D-E, FIG. 8). In contrast, disruption of K11, also an immunomodulatory factor involved in IFN transcription responses⁵¹, shows the opposite phenotype—decreased lytic gene expression and virus production (FIG. 3D-E, FIG. 8). The presence of viral genes which either enhance (e.g. KI 1) or repress (e.g. K6) spontaneous reactivation demonstrates the biological importance of tight viral control over reactivation and implicates these ORFs as critical regulators of latency. Thus, different KSHV immunomodulatory factors affect gene expression to modulate viral reactivation.

### Discussion

This is the first genome-wide study to identify viral genes important for KSHV latency, reactivation, and lytic replication. We found that 44 ORFs are essential for successful completion of the viral life cycle. Of these, 33 ORFs are conserved in all herpesvirus subfamilies, six (ORF10, 18, 24, 30, 31, and 66) are conserved among beta and gamma herpesviruses, and five (ORF45, 50, 52, 73, and 75) are gamma herpesvirus-specific^{2,52}. Surprisingly, 10 ORFs conserved in all herpesvirus subfamilies were found to be nonessential in KSHV (Table 1), despite some of them being essential in other herpesviruses tested (Table S3)^{2,3,53}. These 10 KSHV ORFs, which homologues are essential for the replication of other herpesviruses, may be complemented or substituted by the functions of other KSHV ORFs or cellular genes.

Our profiling results show that reactivation is regulated positively or negatively by two specific sets of viral genes, which may act as important parts of the latent/lytic switch. For example, some ORFs may repress spontaneous (e.g. ORFs 11AA, 72, and K6) or induced reactivation (e.g. K3 and K5) while others (e.g. ORFK11) enhance spontaneous reactivation (FIG. 3, FIG. 5). ORFK11, an IFN modulator, may enhance spontaneous reactivation through changes in interferon responses while ORF72, a constitutively-expressed cyclin homologue, possibly represses spontaneous reactivation through its effects on cell cycle progression. Thus, KSHV encodes specific genes that actively turn on and off reactivation, a critical step for viral lytic replication and pathogenesiss^{5,54-57}.

As an opportunistic pathogen, the onset of KSHV lytic replication and its associated diseases correlate with the host's immune status. One hypothesis is that KSHV engages in random spontaneous reactivation to achieve persistent infection and the host immune responses are responsible for controlling the level of reactivation. However, under immunodeficient conditions, viral reactivation is left unchecked and takes off to full blown lytic replication, leading to KSHV diseases. An alternative hypothesis is that KSHV reactivation is not random but tightly and actively regulated by viral factors, which connect reactivation with the host immune status. It is conceivable that these factors, which regulate reactivation, are involved in sensing, interacting, and modulating immune responses.

The alternative hypothesis is supported by our results. Six ORFs (i.e. K3, K4, K5, K6, K7, and KI 1) known to have immunomodulatory functions were found to promote or suppress virus reactivation and production (Table 1, FIG. 3). Mutants with inactivation in four of these ORFs (i.e. K4, K5, K6, and K11) showed changes in lytic gene expression correlating with changes in virus production (FIG. 3D-E, FIGS. 5-8). These observations further implicate viral immunomodulatory genes in regulating viral reactivation at the gene expression level, even in a cell culture system which lacks adaptive immunity and many innate immunity factors.

K4 and K6 encode viral chemokine homologues^{49,58}. K3 and K5 modulate expression of surface glycoproteins important for immune responses such as MHC and interferon-γ receptor^{45,46,59}. K7 and K11 are anti-apoptotic factors involved in autophagy and IFN transcription responses, respectively^{51,60,61}. These KSHV factors can play a role in modulating the immune-microenvironment, cell membrane receptor composition, and appropriate downstream signaling pathways to produce an immune-switch for KSHV latency and reactivation. The virally reconfigured pathways serve as a sensory network that allows KSHV to communicate with, and deliberately respond to, changes in host homeostasis. In the presence of immuno-selective/repressive pressure, these virally reconstructed pathways promote latency, however, under immunocompromised conditions, these pathways promote lytic replication and progeny production.

### Methods

### Construction of KSHV Mutants.

All annotated KSHV ORFs in the GenBank sequence (accession #GQ994935.1) were selected for mutagenesis, as well as several recently discovered upstream ORFs (uORF)⁹. The BAC mutants were derived from the BAC16 construct using the 2-step RED-mediated recombination methods as described previously 38. For non-overlapping ORFs, the entire ORF from the start to stop codon was deleted from BAC16. For overlapping ORFs, a stop codon sequence (5′-TAGGTAGATAGG-3′) was inserted in a non-overlapping region, downstream of the annotated start codon. The rescued virus was derived from the mutant BAC DNA by restoring the wildtype sequence to the deleted or stop codon-inserted ORF, using the previously described RED-mediated recombination methods³⁸.

The BAC DNAs of the mutants were screened by restriction digest using NheI (Thermo Fisher Scientific, MA, Waltham) to examine the overall BAC genomic structure, and PCR using primers flanking the ORF for the presence of the mutations. The digested and PCR products were separated on agarose gels and visualized on a ChemiDoc Touch apparatus (Bio-Rad Laboratories, CA, Hercules). Sequencing analysis (UC-Berkeley DNA core sequencing facility) also confirmed the stop codon mutations. The primers used for construction and screening of the mutants and rescued viruses are listed in Table S1 and S2.

### Cells and Viruses

KSHV (BAC16), human iSLK cells, and human 293T cells (ATCC, VA, Manassas) were propagated as described previously^{38,43}. Specifically, iSLK cells were maintained in normal/uninduced media, which is Dulbecco's modified eagle's medium (DMEM) with sodium pyruvate and glutamine (Thermo Fisher Scientific, MA, Waltham) supplemented with 10% HI FBS (Cytiva, MA, Marlborough) and 1% Penicillin/Streptomycin (Thermo Fisher Scientific, MA, Waltham). The selection media is DMEM with sodium pyruvate and glutamine supplemented with 10% HI FBS and 1.2 mg/ml hygromycin B (Thermo Fisher Scientific, MA, Waltham). The induction media is DMEM with sodium pyruvate and glutamine supplemented with 10% HI FBS, 1% Penicillin/Streptomycin, 1 ug/ml doxycycline, and 1 mM sodium butyrate.

### Generation of Transfected Cell Lines

BAC DNAs of the mutants were purified using the NucleoBond BAC100 kit (Macherey-Nagel, Germany, Düren) following the manufacturer's instructions, and were used for transfection experiments. Naïve iSLK cells were seeded into 6-well plates at 70-90% confluence (approximately 3.0×10⁵ cells/well), incubated overnight, and then transfected with BAC DNAs (˜2.5 ug/well), using lipofectamine 2000 (Thermo Fisher Scientific, MA, Waltham) following the manufacturer's instructions. At 48 hours post transfection, cells were incubated and expanded in the media containing hygromycin B (1.2 mg/ml). No colony isolations were performed. Cells were monitored by phase and fluorescence microscopy on a Nikon TE300 microscope (Nikon, Japan, Tokyo).

### Generation Viral Stocks

Cells containing the mutant and parental BAC16 DNAs (˜1.7×10⁷ cells) were seeded and then incubated in induction media (DMEM with sodium pyruvate and glutamine supplemented with 10% HI FBS, 1% Penicillin/Streptomycin, 1 ug/ml doxycycline, and 1 mM sodium butyrate) to induce KSHV to reactivate and enter the lytic cycle. At different times post induction, the supernatants were harvested, spun (3,200×g) at 4° C. for 15 minutes, filtered through a 0.45 uM filter (Thermo Scientific Nalgene, MA, Waltham), and concentrated by centrifugation (SureSpin 630 rotor, 13,000 rpm) at 4° C. for 3 hours. The pellet was resuspended in DMEM and stored at −80° C.

### Titration of Viral Stocks

Titration of virus stocks was conducted using 293T cells, following procedures described previously⁶². Briefly, 293T cells seeded in 48-well plates (˜5×10⁴ cells/well) were infected with serial dilutions of virus stocks and then incubated in induction media. After 48 hours the infected cells were examined by fluorescence microscopy using a Nikon TE300 microscope (Nikon, Japan, Tokyo).

The samples with appropriate dilution that contained appropriately 2-20% of GFP+ cells were selected for FACS. Cells were resuspended in 750 ul of “FACS wash buffer” (Dulbecco's phosphate-buffered saline (DPBS) (Thermo Fisher Scientific, MA, Waltham) containing 0.1% w/v BSA (Sigma, MO, St. Louis)) and then fixed in DPBS containing 1% paraformaldehyde (Electron Microscopy Sciences, PA, Hatfield) for 5 minutes at room temperature. The fixed cells were subjected to FACS analysis with a BD-Fortessa X20 cytometer (Becton, Dickinson, NJ, Franklin Lakes). When a mutant cell line yielded no titer, or a very low titer compared to BAC16 cell line, at least two additional independent DNA preparations and transfection were performed to verify the growth phenotype of the mutants. No viral progeny was detected from mutant DNAs containing mutations in essential genes.

### Growth Analysis of KSHV Mutants in Cells

Growth analyses were performed with iSLK cells in 96-well plates. Virus growth was analyzed under three culture conditions. First, under the multi-step growth condition, iSLK cells (˜2.5×10⁴ cells total) were infected with mutants under a multiplicity of infection (MOI) of 0.1, and maintained in induction media (DMEM with sodium pyruvate and glutamine supplemented with 10% HI FBS, 1% Penicillin/Streptomycin, 1 ug/ml doxycycline, and 1 mM sodium butyrate). Supernatants were harvested at 1, 4, 7, 10, 13, 16, and 19 day post-infection (dpi). Second, under the induced reactivation condition, iSLK cells (˜2.5×10⁴ cells total) were infected with mutants (MOI=1). At 2 dpi, cells were incubated in the induction media and supernatants were harvested at 5 dpi. Third, under the spontaneous reactivation condition, iSLK cells (˜2.5×10⁴ cells total) were infected with mutants (MOI=1) and maintained in the normal/uninduced media in the absence of doxycycline and sodium butyrate. Supernatants were harvested at 6 dpi. The supernatants were transferred to new 96-well plates and stored at −80° C. until tittering. Tittering of the supernatants was done as outlined above to determine virus growth at different timepoints. Each analysis was repeated three times and each sample time-point was done in triplicate.

### Immunofluorescence

Mutant and parental BAC16 cell lines were seeded onto coverslips (Corning, NY, Corning) placed in 24-well plates. Cells were either maintained in normal/uninduced media (DMEM with sodium pyruvate and glutamine supplemented with 10% HI FBS, 1% Penicillin/Streptomycin) or induction media (DMEM with sodium pyruvate and glutamine supplemented with 10% HI FBS, 1% Pen Strep, 1 ug/ml doxycycline, and 1 mM sodium butyrate) for 72 hours. Cells were fixed with 4% paraformaldehyde (Electron Microscopy Sciences, PA, Hatfield). Fixed cells were permeabilized with 0.2% Triton X-100 (Sigma, MO, St. Louis) for 10 minutes followed by three 5-minute washes in PBST (DPBS containing 0.1% tween-20 (Sigma, MO, St. Louis)) and a 1-hour incubation in PBST with 5% goat serum (Abcam, UK, Cambridge). Cells were incubated with PBST containing 5% goat serum and anti-LANA antibody (Advanced Biotechnologies, MD, Columbia) followed by incubation with PBST containing 5% goat serum and anti-rat secondary antibody (Life Technologies, CA, Carlsbad). Cells were then incubated with PBST containing 1 ug/ml DAPI (Thermo Fisher Scientific, MA, Waltham) at room temperature, mounted on slides using Fluoromount G (Sigma, MO, St. Louis), and imaged on a Nikon TE300 microscope.

### Flow Cytometry

iSLK cells were trypsinized (Thermo Fisher Scientific, MA, Waltham) and collected by centrifugation at 300×g for 5 minutes at 4° C. Cells were fixed in 4% paraformaldehyde for 5 minutes at room temperature and stored at 4° C. Fixed cells were permeabilized with 0.1% Triton X-100 (Sigma, MO, St. Louis) for 10 minutes at room temperature and blocked for 15 minutes in blocking buffer (DPBS supplemented with 0.5% BSA (Sigma, MO, St. Louis), 0.05% Tween-20 (Sigma, MO, St. Louis), and 5% goat serum (Abcam, UK, Cambridge)). Primary antibody incubation was conducted for 30 minutes with anti-LANA (Advanced Biotechnologies, MD, Columbia), anti-ORF45 (Thermo Fisher Scientific, MA, Waltham), anti-K8 (Promab Biotechnologies, CA, Richmond) or anti-K8.1 (Santa Cruz Biotechnology, TX, Dallas) in blocking buffer. Secondary antibody incubation was conducted for 30-minutes with goat anti-mouse IgG AlexaFluor647 or goat anti-rat IgG AlexaFluor568 (Life Technologies, CA, Carlsbad) in blocking buffer. Cells were analyzed using a BD LSR Fortessa X-20 flow cytometer (Becton Dickinson, Franklin Lakes, NJ) and flowing Software 2.

### Tables

TABLE 1
KSHV ORFs categorized by growth properties of their respective inactivation mutants in
human iSLK cells. The sequence conservations of these ORFs with those in other herpesviruses
of the α, β, γ subfamilies, the genome sequences of which are currently available^{8,63-65}, is
included. ORF functions and the functions of their homologues in other herpesviruses that
have been shown or implicated from previous studies is also shown (Table S3)². ORFs unique
to KSHV are marked as a “U”. D, deletion mutation; S, stop codon mutation.
Essential Genes

| 44 ORFs | ORF | Conservation | Putative Functions |
|---|---|---|---|
| | 73 (D) | γ2 | Tethers viral episomes to chromatin |
| | 6 (D) | α, β, γ | Single-stranded DNA-binding protein^{24,67} |
| | 7 (S) | α, β, γ | Terminase complex |
| | 8 (S) | α, β, γ | Glycoprotein B |
| | 9 (D) | α, β, γ | DNA polymerase^{24,67} |
| | 10 (S) | β, γ | Inhibition of host mRNA nuclear export⁴⁰, derived |
| | | | from ORF54⁶⁸ |
| | 17 (S) | α, β, γ | Maturational protease and capsid scaffolding |
| | | | protein |
| | 18 (S) | β, γ | Late gene expression^{32,69} |
| | 19 (S) | α, β, γ | Portal cap⁷⁰, inner tegument protein⁷¹ |
| | 22 (S) | α, β, γ | Glycoprotein H |
| | 24 (S) | β, γ | Late gene expression^{31,72} |
| | 25 (S) | α, β, γ | Major capsid protein⁷³ |
| | 26 (S) | α, β, γ | Triplex capsid protein⁷³ |
| | 29a (S) | α, β, γ | Terminase complex |
| | 29b (S) | α, β, γ | Terminase complex |
| | 30 (S) | β, γ | Late gene expression^{32,69} |
| | 31 (S) | β, γ | Late gene expression³⁰ |
| | 32 (S) | α, β, γ | Inner tegument protein^{70,71} |
| | 33 (S) | α, β, γ | Tegument protein⁷⁴, egress³⁷ |
| | 34 (S) | α, β, γ | Tegument protein, late gene expression^{30,31} |
| | 39 (D) | α, β, γ | Glycoprotein M |
| | 40 (D) | α, β, γ | Helicase-primase complex⁶⁷ |
| | 41 (S) | α, β, γ | Helicase-primase complex⁶⁷ |
| | 43 (S) | α, β, γ | Portal protein⁷⁰ |
| | 44 (SC) | α, β, γ | Helicase-primase complex⁶⁷ |
| | 45(D) | γ | Tegument protein⁷⁵, egress⁷⁶, reactivation⁷⁷ |
| | 47 (D) | α, β, γ | Glycoprotein L |
| | 50 (D) | γ | RTA, reactivation |
| | 52 (D) | γ | Tegument protein⁷⁸ |
| | 53 (D) | α, β, γ | Glycoprotein N |
| | 55 (S) | α, β, γ | Putative tegument protein |
| | 56 (S) | α, β, γ | Primase⁶⁷ |
| | 57 (D) | α, β, γ | Regulator of gene expression |
| | 59 (D) | α, β, γ | DNA polymerase processivity factor⁶⁷ |
| | 62 (D) | α, β, γ | Triplex capsid protein⁷³ |
| | 63 (D) | α, β, γ | Tegument protein⁷⁴, NLR homolog⁷⁹ |
| | 64 (D) | α, β, γ | Large tegument protein^{70,74} |
| | 65 (D) | α, β, γ | Small capsid protein |
| | 66 (S) | β, γ | Late gene expression⁸⁰ |
| | 67 (S) | α, β, γ | Nuclear egress⁸¹ |
| | 67.5 (S) | α, β, γ | Terminase component (67A) |
| | 68 (S) | α, β, γ | DNA packaging⁸² |
| | 69 (D) | α, β, γ | Nuclear egress⁸¹ |
| | 75 (D) | γ | Phosphoribosylformylglycinamidine synthase |
| | | | (vFAGART), tegument protein⁷⁴ |

Non-Essential Genes (47 ORFs)

| Attenuation | ORF | Conservation | Putative function |
|---|---|---|---|
| Severe (9) | 16 (D) | γ | vBCL-2, antiapoptotic⁸³ |
| | 27 (S) | γ | Putative glycoprotein, tegument protein⁷⁴ |
| | 46 (D) | α, β, γ | Uracil-DNA glycosylase, KHSV late gene expression⁴² |
| | 49 (D) | γ | Co-factor with ORF50 for reactivation⁸⁴ |
| | K8 (D) | U | K-bZIP (KSHV basic leucine zipper)^{67,85,86} |
| | 54 (D) | α, β, γ | Deoxyuridine triphosphatase |
| | 58 (D) | γ | Tegument glycoprotein⁸⁷ |
| | 60 (D) | α, β, γ | Ribonucleotide reductase; small subunit |
| | 61 (D) | α, β, γ | Ribonucleotide reductase; large subunit⁸⁸ |
| Moderate (4) | 4 (D) | γ2 | Kaposica, CD46 homologue for complement |
| | | | modulation⁸⁹ |
| | 35 (S) | γ | Tegument protein⁸⁷ |
| | 36 (S) | α, β, γ | Protein kinase⁹⁰ |
| | 38 (S) | α, β, γ | Egress³⁷ |
| No | K1 (D) | U | KIS (KSHV ITAM signaling protein)^{91,92} |
| Attenuation | 10.1 (S) | U | Unknown function |
| (32) | 11AA (S) | U | Unknown function |
| | 11 (D) | β, γ | Derived from ORF54⁶⁸ |
| | K2 (D) | U | VIL6^{93,94} |
| | 2 (D) | γ2 | Dihydrofolate reductase |
| | K3 (D) | U | MIR-1, immunomodulation^{46,59} |
| | 70 (D) | γ2 | Thymidylate synthase |
| | K4 (D) | U | vMIP-II (vCCL2), immunomodulation^{58,95} |
| | K4.1 (D) | U | vMIP-III (vCCL3), immunomodulation⁹⁶ |
| | K4.2 (S) | U | Unknown function |
| | K5 (D) | U | MIR-2, immunomodulation^{45,46,59} |
| | K6 (D) | U | vMIP-I (vCCL1), immunomodulation⁴⁹ |
| | K7 (S) | U | vIAP, antiapoptotic^{60,61} |
| | 20 (S) | α, β, γ | Nuclear protein, cell cycle arrest⁹⁷ |
| | 21 (S) | α, γ | Thymidine kinase, tegument protein⁷⁴ |
| | 23 (S) | α, β, γ | Late gene expression¹⁸, tegument protein⁸⁷ |
| | 30.1 (S) | U | Unknown function |
| | 34.1 (S) | U | Unknown function |
| | 37 (S) | α, β, γ | Deoxyribonuclease; DNA maturation and |
| | | | recombination |
| | 42 (S) | α, β, γ | Tegument protein⁸⁷ |
| | 48 (D) | γ | Tegument protein⁸⁷ |
| | K9 (S) | U | vIRF1⁹⁸, anti-apoptotic⁹⁹ |
| | K10 (D) | U | vIRF4, anti-apoptotic¹⁰⁰ |
| | K10.5 (D) | U | vIRF-3/LANA2, anti-apoptotic^{101,102} |
| | K11 (S) | U | vIRF-2⁵¹, anti-apoptotic¹⁰³ |
| | K12 (D) | U | Kaposin A^{104,105} |
| | K13/71 (D) | U | vFLIP (FLICE [FADD-like interleukin1 beta- |
| | | | converting enzyme, now called caspase8] inhibitory |
| | | | protein), anti-apoptosis, immunomodulation¹⁰⁶⁻¹⁰⁸ |
| | 72 (D) | γ2 | vCyclin^{109,110} |
| | K14 (D) | U | vOX2, immunomodulation¹¹¹ |
| | 74 (D) | γ2 | vGPCR¹¹² |
| | K15 (D) | U | anti-apoptotic, immunomodulation^{113,114} |
| Enhanced | 28 (D) | γ | Envelope glycoprotein^{74,115} |
| Growth (2) | K8.1 (D) | U | Envelope glycoprotein¹⁶ |

TABLE S1
Primers for KSHV mutagenesis and PCR. For each ORF, forward
primers are listed in the top row and reverse primers in the bottom row.

| | Deletion/Insertion Primers | PCR Primers |
|---|---|---|
| ORF | (5′→3′) | (5′→3′) |
| K1 | CCTGTCTTTCAGACCTTGTTGGACATCCCGTACAAT | CGGCCCTTGTGT |
| | CAAGCATTCAGGTAAGATAATCTAAGGATGACGAC | AAACCTGTC |
| | GATAAGTAGGG (SEQ ID NO: 93) | (SEQ ID NO: 1) |
| | ATTATGTTATAGAGAATATTTAGATTATCTTACCTG | GCACGGTTATAC |
| | AATGCTTGATTGTACGGGATGTCCAACCAATTAAC | AATGTCCT |
| | CAATTCTGATTAG (SEQ ID NO: 94) | (SEQ ID NO: 1) |
| 2 | CTAACGCGGCATACACTAGCCGGTGGTGCCCGAGC | AGGACATTGTAT |
| | GGGAGGCCGCGAGGGTATAGGTAAAAGGATGACG | AACCGTGC |
| | ACGATAAGTAGGG(SEQ ID NO: 95) | (SEQ ID NO: 2) |
| | ACACTGTGTGGTTGGTGGTGTTTACCTATACCCTCG | GTTGTCTTGTATT |
| | CGGCCTCCCGCTCGGGCACCACCGAACCAATTAAC | GGTCGGT |
| | CAATTCTGATTAG (SEQ ID NO: 96) | (SEQ ID NO: 2) |
| 4 | GTACATTAAAAGGACATTGTATAACCGTGCTACTT | TATAGTGCGCGG |
| | ACAGCCCTAGACTTGCTCCAGTGTTAGGATGACGA | TGTGGCAG |
| | CGATAAGTAGGG (SEQ ID NO: 97) | (SEQ ID NO: 3) |
| | AAGCAATCATAGCCCTGTCTAACACTGGAGCAAGT | GAGTAGTGTGCC |
| | CTAGGGCTGTAAGTAGCACGGTTATAACCAATTAA | GTGAAGGCT |
| | CCAATTCTGATTAG (SEQ ID NO: 98) | (SEQ ID NO: 3) |
| 6 | TACACACGGGTTTTTTGTTGTCTTGGCCAATCGTGT | ACAGTCGGTAGT |
| | CTCCTTGTGTACCCGTAACGATGGAGGATGACGAC | GGAGGAGC |
| | GATAAGTAGGG | (SEQ ID NO: 4) |
| | (SEQ ID NO: 99) | |
| | GACCGCCGCCAGTTCCTTTGCCATCGTTACGGGTAC | GCTCTGAAACTT |
| | ACAAGGAGACACGATTGGCCAAGAAACCAATTAA | CCCTGTAGTGA |
| | CCAATTCTGATTAG (SEQ ID NO: 100) | (SEQ ID NO: 4) |
| 7 | GACCTGGATTTGTAGTTGTGTACCCGTAACGATGG | GAGGAACCGAAA |
| | CAAAGTAGGTAGATAGGGAACTGGCGGCGGTCTAT | CCCGCAGG |
| | GCAGGATGACGACGATAAGTAGGG (SEQ ID NO: | (SEQ ID NO: 5) |
| | 101) | |
| | TGGCTAGGGCTGACACATCGGCATAGACCGCCGCC | CCACACTCTTAG |
| | AGTTCCCTATCTACCTACTTTGCCATCGTTACGGGT | GACCAGATGCTT |
| | AAACCAATTAACCAATTCTGATTAG (SEQ ID NO: | (SEQ ID NO: 5) |
| | 102) | |
| 8 | CTGTACCACCACCTGCAATTGAGCAACCACAATGA | CAATCGCTAGAC |
| | CTCCCTAGGTAGATAGGAGGTCTAGATTGGCCACC | ATCAGTCC |
| | CTAGGATGACGACGATAAGTAGGG (SEQ ID NO: | (SEQ ID NO: 6) |
| | 103) | |
| | CCAACAGGATGACAGTCCCCAGGGTGGCCAATCTA | CGTTATCTCCCA |
| | GACCTCCTATCTACCTAGGGAGTCATTGTGGTTGCT | GTCACCTA |
| | CAACCAATTAACCAATTCTGATTAG (SEQ ID NO: | (SEQ ID NO: 6) |
| | 104) | |
| 9 | CTACTCGTTACACACAGACACAAATTACCGTCCGC | AGAACAACACGT |
| | AGATCTGACTCAGACGCGGAAACAGAGGATGACG | CGGCAACC |
| | ACGATAAGTAGGG (SEQ ID NO: 105) | (SEQ ID NO: 7) |
| | AAGAGGAAACTTTCTAGGCGCTGTTTCCGCGTCTG | GGATTCTTAGCC |
| | AGTCAGATCTGCGGACGGTAATTTGAACCAATTAA | GCGTGTAGT |
| | CCAATTCTGATTAG (SEQ ID NO: 106) | (SEQ ID NO: 7) |
| 10.1 | CTTGCGCTATGTGGGACAACTAGAGTCCAACCTGG | CCACACTCTTAG |
| | CAAGCTAGGTAGATAGGAGTGGAGCAAGACGCCA | GACCAGATGCTT |
| | GACAGGATGACGACGATAAGTAGGG(SEQ ID NO: | (SEQ ID NO: 8) |
| | 107) | |
| | TATTTTTTTCGAGATCGGCTGTCTGGCGTCTTGCTC | CCACACTCTTAG |
| | CACTCCTATCTACCTAGCTTGCCAGGTTGGACTCTA | GACCAGATGCTT |
| | AACCAATTAACCAATTCTGATTAG (SEQ ID NO: 108) | (SEQ ID NO: 8) |
| 10 | AACGTTCATCCTAGGTGACTGGGAGATAACGGTGT | GCCAGGCACCAT |
| | CTAACTAGGTAGATAGGTGCCGGTTTACTTGCAGC | ACAGCTTC |
| | AGAGGATGACGACGATAAGTAGGG (SEQ ID NO: | (SEQ ID NO: 9) |
| | 109) | |
| | AAAGGGGGCCACATGTTAGGCTGCTGCAAGTAAAC | GATTAGAGATGA |
| | CGGCACCTATCTACCTAGTTAGACACCGTTATCTCC | CGATGTGGCTCG |
| | CAACCAATTAACCAATTCTGATTAG (SEQ ID NO: | G |
| | 110) | (SEQ ID NO: 9) |
| 11AA | CCACGTAGCGATTAGGGCCGACCGCCACGAGGAAC | CAGCTTCTACGA |
| | CCATGTAGGTAGATAGGCAATCGTGACTGTCCGAG | CTGCAAGG |
| | CAAGGATGACGACGATAAGTAGGG(SEQ ID NO: | (SEQ ID NO: 10) |
| | 111) | |
| | TCTGACTCCTGCGCCATATGTGCTCGGACAGTCAC | GTGTGTGTTATG |
| | GATTGCCTATCTACCTACATGGGTTCCTCGTGGCGG | TATTCGGGTGG |
| | TAACCAATTAACCAATTCTGATTAG (SEQ ID NO: | (SEQ ID NO: 10) |
| | 112) | |
| 11 | GCCACGAGGAACCCATGCAATCGTGACTGTCCGAG | TGTTCCCATACG |
| | CACATGTGTCCGGTTCCCACCCACAAGGATGACGA | CGCCTGTC |
| | CGATAAGTAGGG (SEQ ID NO: 113) | (SEQ ID NO: 11) |
| | GAAAGCAATAAAGACAAATGTGTGGGTGGGAACC | CTGGCGAGCAAG |
| | GGACACATGTGCTCGGACAGTCACGAAACCAATTA | AGAGGGTTT |
| | ACCAATTCTGATTAG (SEQ ID NO: 114) | (SEQ ID NO: 11) |
| K2 | GTATATTAGTGTTATAAGAAATTTTATGTCACGTCG | CGCGTTCCAGAT |
| | CTCTGGCTGCTAACGCGGCATACAAGGATGACGAC | ACCAGCAG |
| | GATAAGTAGGG (SEQ ID NO: 115) | (SEQ ID NO: 12) |
| | GCTCGGGCACCACCGGCTAGTGTATGCCGCGTTAG | GCTGGACCCTCC |
| | CAGCCAGAGCGACGTGACATAAAATAACCAATTAA | TCTCTAGTT |
| | CCAATTCTGATTAG (SEQ ID NO: 116) | (SEQ ID NO: 12) |
| 2 | CTAACGCGGCATACACTAGCCGGTGGTGCCCGAGC | TGGTATCAACCG |
| | GGGAGGCCGCGAGGGTATAGGTAAAAGGATGACG | CAACTACACAG |
| | ACGATAAGTAGGG (SEQ ID NO: 117) | (SEQ ID NO: 13) |
| | ACACTGTGTGGTTGGTGGTGTTTACCTATACCCTCG | GTGAGTGGCTGT |
| | CGGCCTCCCGCTCGGGCACCACCGAACCAATTAAC | AGCATTACC |
| | CAATTCTGATTAG (SEQ ID NO: 118) | (SEQ ID NO: 13) |
| K3 | GGTAAACACCACCAACCACACAGTGTGCTCTTATA | GATTCATGTAGA |
| | TACTTATCCTGAGAGAGAACCCACAAGGATGACGA | CAACCCGCTC |
| | CGATAAGTAGGG (SEQ ID NO: 119) | (SEQ ID NO: 14) |
| | GGGTTAATGCCATGTTTTATTGTGGGTTCTCTCTCA | CGTGGGAACTGT |
| | GGATAAGTATATAAGAGCACACTGAACCAATTAAC | GAGTAATGTG |
| | CAATTCTGATTAG (SEQ ID NO: 120) | (SEQ ID NO: 14) |
| 70 | GCCCACCACTCTGACCGCACGCTAAACATCGCCCT | CGCTGTTCTCCTG |
| | ACCTGGATAGATCCTGGAAGTTTGTAGGATGACGA | TAATTGG |
| | CGATAAGTAGGG (SEQ ID NO: 121) | (SEQ ID NO: 15) |
| | CTCTCCGGGCACAGGGCTTCACAAACTTCCAGGAT | GGTGTTGGCCTT |
| | CTATCCAGGTAGGGCGATGTTTAGCAACCAATTAA | CGTAATAA |
| | CCAATTCTGATTAG (SEQ ID NO: 122) | (SEQ ID NO: 15) |
| K4 | AGAGATCCGTCGCGTAAATGCGCAGCTGGCAAAGC | ACGACGGTTACA |
| | ATTCTAACTCCCCTCGTGTGTCCTCAGGATGACGAC | GGTCCCTC |
| | GATAAGTAGGG (SEQ ID NO: 123) | (SEQ ID NO: 16) |
| | TCGCCGTTTCGCATTTACACGAGGACACACGAGGG | CATTTGGCAACG |
| | GAGTTAGAATGCTTTGCCAGCTGCGAACCAATTAA | CTGGTCTT |
| | CCAATTCTGATTAG (SEQ ID NO: 124) | (SEQ ID NO: 16) |
| K4.1 | TATGTCACAGACTCAACACACACGGGCCGTTACGC | CTGGCCGCAATA |
| | AACGGACAGTTCTGGCGCCACAACGAGGATGACG | GCTCAATC |
| | ACGATAAGTAGGG (SEQ ID NO: 125) | (SEQ ID NO: 17) |
| | TGCACCCCTTTGCGCATCATCGTTGTGGCGCCAGA | CCGCTAACAGCA |
| | ACTGTCCGTTGCGTAACGGCCCGTGAACCAATTAA | CCAAATCCAC |
| | CCAATTCTGATTAG (SEQ ID NO: 126) | (SEQ ID NO: 17) |
| K4.2 | ACATAATTTATGCACATAAAAGGATTAGCGCATGC | AGGACAGATTTG |
| | AAATTTAGGTAGATAGGAGCTTTGCCGAAGTTCTC | GGCACAGG |
| | GGAGGATGACGACGATAAGTAGGG (SEQ ID NO: | (SEQ ID NO: 18) |
| | 127) | |
| | CAGCGCGCCCCACCGGCTTTCCGAGAACTTCGGCA | CAGGGTGGGTTG |
| | AAGCTCCTATCTACCTAAATTTGCATGCGCTAATCC | TGACAGTT |
| | TAACCAATTAACCAATTCTGATTAG (SEQ ID NO: | (SEQ ID NO: 18) |
| | 128) | |
| K5 | GGGCGTCACGTCACATATCTCTGTGCACCCAAGTG | CATTTCTTCCTCG |
| | GTTGTCTCTGCAGCTGGGGTGGAAGAGGATGACGA | ACAGGTCTTC |
| | CGATAAGTAGGG (SEQ ID NO: 129) | (SEQ ID NO: 19) |
| | TCCCCTTTCCCTTTTTCAGACTTCCACCCCAGCTGC | GCATGTAAGCTG |
| | AGAGACAACCACTTGGGTGCACAGAACCAATTAAC | GCGGTTAG |
| | CAATTCTGATTAG (SEQ ID NO: 130) | (SEQ ID NO: 19) |
| K6 | GAGCAGTTGGGCCGCAGTGATATCTTCAACTTTCG | TTATGGATTATT |
| | ACCGTCTGGAGGTGCCAAGTTCGCAAGGATGACGA | AAGGGTCAGCTT |
| | CGATAAGTAGGG (SEQ ID NO: 131) | G |
| | | (SEQ ID NO: 20) |
| | TACGGTTTTCTTTAGACTGTTGCGAACTTGGCACCT | CGACGCAATCAA |
| | CCAGACGGTCGAAAGTTGAAGATAAACCAATTAAC | CCCACAAT |
| | CAATTCTGATTAG (SEQ ID NO: 132) | (SEQ ID NO: 20) |
| K7 | TCCAAAAATGGGTGGCTAACCTGTCCAAAATATGG | GGAACCAGCTTG |
| | GAACATAGGTAGATAGGCTGGAGATAAAAGGGGC | GTGATGTG |
| | CAGAGGATGACGACGATAAGTAGGG (SEQ ID NO: | (SEQ ID NO: 21) |
| | 133) | |
| | CAGTGCTAAACTGACTCAAGCTGGCCCCTTTTATCT | CAAGCAGTAGCG |
| | CCAGCCTATCTACCTATGTTCCCATATTTTGGACAG | AACAGTTACG |
| | AACCAATTAACCAATTCTGATTAG (SEQ ID NO: 134) | (SEQ ID NO: 21) |
| 16 | GGTGCTGTGCGCGTGCTATGTTCCCTGGTGACCGTC | GGCAGGACACAA |
| | CACACGCGTAATTCGAGGTCCCCGAGGATGACGAC | CATCTACAAAC |
| | GATAAGTAGGG (SEQ ID NO: 135) | (SEQ ID NO: 22) |
| | CATGCAACCATCTACTCTTCCGGGGACCTCGAATT | CATTGGCAGTAG |
| | ACGCGTGTGGACGGTCACCAGGGAAAACCAATTAA | CCTCCCTTAA |
| | CCAATTCTGATTAG (SEQ ID NO: 136) | (SEQ ID NO: 22) |
| 17 | CTCGGTCTCACACACGTATTTTCCGAGCATGGCAC | TACCGTGGGACA |
| | AGGGCTAGGTAGATAGGCTGTACGTCGGAGGGTTT | CTTGAAATAGAC |
| | GTAGGATGACGACGATAAGTAGGG (SEQ ID NO: | (SEQ ID NO: 23) |
| | 137) | |
| | TGGGGCAGGACACAACATCTACAAACCCTCCGACG | GCTCTTGGGCGT |
| | TACAGCCTATCTACCTAGCCCTGTGCCATGCTCGGA | GGAATGTA |
| | AAACCAATTAACCAATTCTGATTAG (SEQ ID NO: | (SEQ ID NO: 23) |
| | 138) | |
| 18 | GCCGCTGAGCCCGGGGCTTAGGAGGCTCATGTGGC | CATTACATCGCT |
| | GCTTTTAGGTAGATAGGTTGCAAAATAAGAATTTA | CACTCGGCCC |
| | AAAGGATGACGACGATAAGTAGGG (SEQ ID NO: | (SEQ ID NO: 24) |
| | 139) | |
| | GCTCTTGGGCGTGGAATGTATTTAAATTCTTATTTT | CGGTGAAGTTAC |
| | GCAACCTATCTACCTAAAAGCGCCACATGAGCCTC | GGTGGCTG |
| | CAACCAATTAACCAATTCTGATTAG (SEQ ID NO: | (SEQ ID NO: 24) |
| | 140) | |
| 19 | GGACCGGCTTGTTCAGGTCCATGACTCACGCGTCC | GATCCATTGGCG |
| | GCGTCTAGGTAGATAGGATTAACGCAGATATCGAC | CGTAGTCTC |
| | GCAGGATGACGACGATAAGTAGGG (SEQ ID NO: | (SEQ ID NO: 25) |
| | 141) | |
| | TGCCTATCATCTGTTTCACCGCGTCGATATCTGCGT | AGCTCGGACGAC |
| | TAATCCTATCTACCTAGACGCGGACGCGTGAGTCA | GAATCAAG |
| | TAACCAATTAACCAATTCTGATTAG (SEQ ID NO: | (SEQ ID NO: 25) |
| | 142) | |
| 20 | CGAGTCCGCTCCAAAACCGCCTTCTGCCATGGTAC | CGGCAATTCTGT |
| | GTCCATAGGTAGATAGGACCGAGGCCGAGGTTAAG | GCCCTAGAG |
| | AAAGGATGACGACGATAAGTAGGG (SEQ ID NO: | (SEQ ID NO: 26) |
| | 143) | |
| | CTGGAAGCCTGCTCAGGGATTTCTTAACCTCGGCCT | GTGCTTGATTCG |
| | CGGTCCTATCTACCTATGGACGTACCATGGCAGAA | TCGTCCGAG |
| | GAACCAATTAACCAATTCTGATTAG (SEQ ID NO: | (SEQ ID NO: 26) |
| | 144) | |
| 21 | TTTCTTAACCTCGGCCTCGGTTGGACGTACCATGGC | CCCGTCGTGATC |
| | AGAATAGGTAGATAGGGGCGGTTTTGGAGCGGACT | GAGCTTTG |
| | CAGGATGACGACGATAAGTAGGG (SEQ ID NO: 145) | (SEQ ID NO: 27) |
| | TTTCTCCGCCGCGCCCCACCGAGTCCGCTCCAAAA | GGAGCGGGTTAT |
| | CCGCCCCTATCTACCTATTCTGCCATGGTACGTCCA | TGTCGTCG |
| | AAACCAATTAACCAATTCTGATTAG (SEQ ID NO: | (SEQ ID NO: 27) |
| | 146) | |
| 22 | AGTCTAAAGCAGTTAATCACCTAGAGGAGACATGC | GTCATCGGTTCG |
| | AGGGTTAGGTAGATAGGCTAGCCTTCTTGGCGGCC | CCGCTCTA |
| | CTAGGATGACGACGATAAGTAGGG (SEQ ID NO: | (SEQ ID NO: 28) |
| | 147) | |
| | ATATGCATCGCCAGCATGCAAGGGCCGCCAAGAAG | GAACAACAGTGG |
| | GCTAGCCTATCTACCTAACCCTGCATGTCTCCTCTA | CATCGGGAC |
| | GAACCAATTAACCAATTCTGATTAG (SEQ ID NO: | (SEQ ID NO: 28) |
| | 148) | |
| 23 | CGTACGTTGCGTCCGTCCGCTGGTCTAAGCTATGTT | GGACACTGCCTT |
| | ACGATAGGTAGATAGGGTTCCGGACGTGAAGGCTA | CTCTGGCG |
| | GAGGATGACGACGATAAGTAGGG (SEQ ID NO: 149) | (SEQ ID NO: 29) |
| | GCGCCGCGCCCTCTACTAGACTAGCCTTCACGTCC | CTTCTAAGGTCA |
| | GGAACCCTATCTACCTATCGTAACATAGCTTAGAC | GCTCTGCCTGC |
| | CAAACCAATTAACCAATTCTGATTAG (SEQ ID NO: | (SEQ ID NO: 29) |
| | 150) | |
| 24 | CGGGAAAGGTCGTTGCTCCAAGGTCGCCTCCATGG | AGTAGTCTGCGT |
| | CAGCGTAGGTAGATAGGCTCGAGGGCCCCCTACTA | ATCGCTCTGC |
| | CTAGGATGACGACGATAAGTAGGG (SEQ ID NO: | (SEQ ID NO: 30) |
| | 151) | |
| | TCAGGGAGGCGCTCGGTGGCAGTAGTAGGGGGCCC | CCTTGCCGAGCA |
| | TCGAGCCTATCTACCTACGCTGCCATGGAGGCGAC | ATAGCTGAAA |
| | CTAACCAATTAACCAATTCTGATTAG (SEQ ID NO: | (SEQ ID NO: 30) |
| | 152) | |
| 25 | TGGCAGTAGTAGGGGGCCCTCGAGCGCTGCCATGG | CGTCGTGGTCAA |
| | AGGCGTAGGTAGATAGGACCTTGGAGCAACGACCT | CGGTACAG (SEQ |
| | TTAGGATGACGACGATAAGTAGGG (SEQ ID NO: | ID NO: 31) |
| | 153) | |
| | CCTCCGTGGCGAGGTACGGGAAAGGTCGTTGCTCC | CAATCTCCGAGC |
| | AAGGTCCTATCTACCTACGCCTCCATGGCAGCGCT | GGCAGTAC (SEQ |
| | CGAACCAATTAACCAATTCTGATTAG (SEQ ID NO: | ID NO: 31) |
| | 154) | |
| 26 | TATTAGCTAACCCTTCTAGCGTTGGCTAGTCATGGC | TCTGGACGTAGA |
| | ACTCTAGGTAGATAGGGACAAGAGTATAGTGGTTA | CAACACGGATC |
| | AAGGATGACGACGATAAGTAGGG (SEQ ID NO: 155) | (SEQ ID NO: 32) |
| | CGAAGAGTCTGGAGGTGAAGTTAACCACTATACTC | GAGCCGTCATCC |
| | TTGTCCCTATCTACCTAGTTGGCTAGTCATGGCACT | GTCCTTGC |
| | CAACCAATTAACCAATTCTGATTAG (SEQ ID NO: | (SEQ ID NO: 32) |
| | 156) | |
| 27 | GAGACTTTGGCGGCCTCCTGTTGGTATTCCCCACGC | TTTACGCTAAGA |
| | TAACTAGGTAGATAGGGATTTGAAGCGGGGGGGG | GTTGGGTGCTTG |
| | GGAGGATGACGACGATAAGTAGGG (SEQ ID NO: | (SEQ ID NO: 33) |
| | 157) | |
| | GAATATCAGATGACGCCATACCCCCCCCCCGCTTC | GGACGACTTACT |
| | AAATCCCTATCTACCTAGTTAGCGTGGGGAATACC | TGTGGCAGT |
| | AAAACCAATTAACCAATTCTGATTAG (SEQ ID NO: | (SEQ ID NO: 33) |
| | 158) | |
| 28 | CTCAGTTGAGAGTCAGAGAATACAGTGCTAATCAG | GTTCGCCTCCTCT |
| | GGTAGAACGGGGTGTGTGCTATAATAGGATGACGA | CCTTTACTGTTA |
| | CGATAAGTAGGG (SEQ ID NO: 159) | (SEQ ID NO: 34) |
| | TACAGTGCTAATCAGGGTAGAGCCCCCCCCATAGC | GGTGGAAATCTT |
| | CATCCATTATAGCACACACCCCGTTAACCAATTAA | CGCGGTGG |
| | CCAATTCTGATTAG (SEQ ID NO: 160) | (SEQ ID NO: 34) |
| 29B | AAAGGATGCACTGCCGGCTATTCTGGGTTTCATGC | TGGGCGTTCCTG |
| | TTCAGTAGGTAGATAGGAAAGACGCCAAGCTTATA | AGGTTAAG |
| | TTAGGATGACGACGATAAGTAGGG (SEQ ID NO: | (SEQ ID NO: 35) |
| | 161) | |
| | ACGAGTTCACGGATGATATAAATATAAGCTTGGCG | CCAAGAACAAGA |
| | TCTTTCCTATCTACCTACTGAAGCATGAAACCCAGA | GCCACGCA |
| | AAACCAATTAACCAATTCTGATTAG (SEQ ID NO: | (SEQ ID NO: 35) |
| | 162) | |
| 30.1 | TACGTAGAGCAGGTTAAAGGTCTGTCCCCGAATGC | TCTGAAGCATGA |
| | TCTGCTAGGTAGATAGGAGACACGGAAAGACACA | AACCCAGAATAG |
| | AAAAGGATGACGACGATAAGTAGGG (SEQ ID NO: | (SEQ ID NO: 36) |
| | 163) | |
| | TAGCCGCTTATGAGCCCCTCTTTTGTGTCTTTCCGT | CGACCACTTGCT |
| | GTCTCCTATCTACCTAGCAGAGCATTCGGGGACAG | CCCTAAGG |
| | AAACCAATTAACCAATTCTGATTAG (SEQ ID NO: | (SEQ ID NO: 36) |
| | 164) | |
| 30 | GGGAATAAAAGGGGGCGTGTGTGCCGATCGTATGG | CAGTAAAGGAGA |
| | GTGAGTAGGTAGATAGGCCAGTGGATCCTGGACAT | GGAGGCGAAC |
| | GTAGGATGACGACGATAAGTAGGG (SEQ ID NO: | (SEQ ID NO: 37) |
| | 165) | |
| | CAAAATCTTTCTCATTCACCACATGTCCAGGATCCA | GGACTCGCCACA |
| | CTGGCCTATCTACCTAGTGCCGATCGTATGGGTGA | CTCTTCATT |
| | GAACCAATTAACCAATTCTGATTAG (SEQ ID NO: | (SEQ ID NO: 37) |
| | 166) | |
| 31 | TGTGCCGCCTAGACACCGGTGCGAAATGAAGAGTG | CGTGCAGGAAAT |
| | TGGCGTAGGTAGATAGGAGTCCCTTATGTCAGTTC | AGCCCTGG |
| | CAAGGATGACGACGATAAGTAGGG (SEQ ID NO: | (SEQ ID NO: 38) |
| | 167) | |
| | GGTACAGGCAAAACACGCCGTGGAACTGACATAA | GTGATGCAGCAG |
| | GGGACTCCTATCTACCTACGCCACACTCTTCATTTC | AAAGGTCCT |
| | GCAACCAATTAACCAATTCTGATTAG (SEQ ID NO: | (SEQ ID NO: 38) |
| | 168) | |
| 32 | ACGACTGCATTGCCAAGCGGGTGCGGACAAAATGG | ACGGACGGTGAC |
| | ATGCGTAGGTAGATAGGCATGCTATCAACGAAAGA | TGGTTAGAG |
| | TAAGGATGACGACGATAAGTAGGG (SEQ ID NO: | (SEQ ID NO: 39) |
| | 169) | |
| | GGTGGCAGCGAGGACCTACGTATCTTTCGTTGATA | GAGATAGAGGTG |
| | GCATGCCTATCTACCTAGTGCGGACAAAATGGATG | CAGGCGTTAAAG |
| | CGAACCAATTAACCAATTCTGATTAG (SEQ ID NO: | (SEQ ID NO: 39) |
| | 170) | |
| 33 | AAGGAGGATCTGGTGTTCATTCGAGGCCGCTATGG | ACGGACGGTGAC |
| | CTAGCTAGGTAGATAGGCGGAGGCGCAAACTTCGG | TGGTTAGAG |
| | AAAGGATGACGACGATAAGTAGGG (SEQ ID NO: | (SEQ ID NO: 40) |
| | 171) | |
| | TGCATTCCTTGTTTAGGAAATTCCGAAGTTTGCGCC | GAGATAGAGGTG |
| | TCCGCCTATCTACCTAGCTAGCCATAGCGGCCTCG | CAGGCGTTAAAG |
| | AAACCAATTAACCAATTCTGATTAG (SEQ ID NO: | (SEQ ID NO: 40) |
| | 172) | |
| 29A | ACCCTCGGACACGAGCGAGCTCAAAGCAAACATGC | CGTTGTCCTCGG |
| | TGCTCTAGGTAGATAGGAGCCGTCACAGGGAGCGC | ACGGTCTG |
| | CTAGGATGACGACGATAAGTAGGG (SEQ ID NO: | (SEQ ID NO: 41) |
| | 173) | |
| | TCTCCTGCAGGTTGGCGGCAAGGCGCTCCCTGTGA | CTACTGGTCACC |
| | CGGCTCCTATCTACCTAGAGCAGCATGTTTGCTTTG | TCCGGGTCA |
| | AAACCAATTAACCAATTCTGATTAG (SEQ ID NO: | (SEQ ID NO: 41) |
| | 174) | |
| 34.1 | ATCCGTGCCGTTTTGGGACAGTGTCGCGTGAATGT | GATTTGCTGACG |
| | CGGGGTAGGTAGATAGGCACTCAGTTCCCACCTCT | TGGGCGTG |
| | CTAGGATGACGACGATAAGTAGGG (SEQ ID NO: | (SEQ ID NO: 42) |
| | 175) | |
| | GAGACCGCCAAAGACGCCGGAGAGAGGTGGGAAC | CCAGGTGTGTTC |
| | TGAGTGCCTATCTACCTACCCCGACATTCACGCGA | TCGCTAAGGT |
| | CACAACCAATTAACCAATTCTGATTAG (SEQ ID NO: | (SEQ ID NO: 42) |
| | 176) | |
| 34 | GGCAAGGCGCTCCCTGTGACGGCTGAGCAGCATGT | TTACATTTCCCAC |
| | TTGCTTAGGTAGATAGGTTGAGCTCGCTCGTGTCCG | ACCTGCCTC |
| | AAGGATGACGACGATAAGTAGGG (SEQ ID NO: 177) | (SEQ ID NO: 43) |
| | TGGTCACCTCCGGGTCACCCTCGGACACGAGCGAG | CTCAAACACGGC |
| | CTCAACCTATCTACCTAAGCAAACATGCTGCTCAG | GCTGCTAC |
| | CCAACCAATTAACCAATTCTGATTAG (SEQ ID NO: | (SEQ ID NO: 43) |
| | 178) | |
| 35 | ATACCGGCGATCATCACCATGATCAAGGAGAATGG | AGTTTCAGGACG |
| | ACTCATAGGTAGATAGGACCAACTCTAAAAGAGAG | CACAGCATG |
| | TTAGGATGACGACGATAAGTAGGG (SEQ ID NO: | (SEQ ID NO: 44) |
| | 179) | |
| | CCTCCAGAGCCGACTTAATAAACTCTCTTTTAGAGT | GCGCTTTAAGAT |
| | TGGTCCTATCTACCTATGAGTCCATTCTCCTTGATC | ACGCGGTGG |
| | AACCAATTAACCAATTCTGATTAG (SEQ ID NO: 180) | (SEQ ID NO: 44) |
| 36 | TTGCCCCCGGTGTGCCCTGAAACTCCCTAAGGCTA | GATTCCCATGCG |
| | CCCGGTAGGTAGATAGGATTTCAGAGAGACCCTGG | ACAGGAGC |
| | GCAGGATGACGACGATAAGTAGGG (SEQ ID NO: | (SEQ ID NO: 45) |
| | 181) | |
| | GATTCAGCTGCCATGTGGACGCCCAGGGTCTCTCT | CAGTCTCGAACC |
| | GAAATCCTATCTACCTACCGGGTAGCCTTAGGGAG | TTGGCGTG |
| | TTAACCAATTAACCAATTCTGATTAG (SEQ ID NO: | (SEQ ID NO: 45) |
| | 182) | |
| 37 | AGAAAGCTACTAGAGCGAGACTTTTTCAACCATGG | CCTGTCAACTGT |
| | AGGCCTAGGTAGATAGGACCCCCACACCCGCGGAC | ACCATCGGTG |
| | TTAGGATGACGACGATAAGTAGGG(SEQ ID NO: 183) | (SEQ ID NO: 46) |
| | CCAGATAGTCTTCAGAAAACAAGTCCGCGGGTGTG | GGATTGCGATTG |
| | GGGGTCCTATCTACCTAGGCCTCCATGGTTGAAAA | CTCAAGCA |
| | AGAACCAATTAACCAATTCTGATTAG (SEQ ID NO: | (SEQ ID NO: 46) |
| | 184) | |
| 38 | GAAGTGCGAAGGACACCTTTCCATATATCAAATGG | GGGATGGAGGAA |
| | GATTTTAGGTAGATAGGCTCCTATCTATCTGCAAAC | GAGGGATG |
| | GAGGATGACGACGATAAGTAGGG (SEQ ID NO: 185) | (SEQ ID NO: 47) |
| | CGTCTACGGGCTGTGAGGGACGTTTGCAGATAGAT | GGACGTGAACGC |
| | AGGAGCCTATCTACCTAAAATCCCATTTGATATAT | TGTGAAAG (SEQ |
| | GGAACCAATTAACCAATTCTGATTAG (SEQ ID NO: | ID NO: 47) |
| | 186) | |
| 39 | ATGGAGGAAGAGGGATGGGTTTATAATGCCAATAT | GCGGGAGAGCCA |
| | ATCAGGTTTCTCGGTCTTTTTAACTAGGATGACGAC | ATCTGATG |
| | GATAAGTAGGG (SEQ ID NO: 187) | (SEQ ID NO: 48) |
| | CCGCAGCGCCGCCTGGCGAAAGTTAAAAAGACCG | CCTTTAGAGTAA |
| | AGAAACCTGATATATTGGCATTATAAAACCAATTA | ACCCGGCCATC |
| | ACCAATTCTGATTAG (SEQ ID NO: 188) | (SEQ ID NO: 48) |
| 40 | GCCCCGGGCAGAAGCCAGAGGTAGTCGACTCATTG | AGGTGAGACCTA |
| | ACTCAAGCGGAGAGGGGGTGGTGCGAGGATGACG | CTGTCCCTG |
| | ACGATAAGTAGGG (SEQ ID NO: 189) | (SEQ ID NO: 49) |
| | AAACCCGTCAACTGCCAACTCGCACCACCCCCTCT | GGTGATGTGACG |
| | CCGCTTGAGTCAATGAGTCGACTACAACCAATTAA | TGGGTTAGG |
| | CCAATTCTGATTAG (SEQ ID NO: 190) | (SEQ ID NO: 49) |
| 41 | AGAATTCAAGGATCTCAAAAGGGCCTGCCAGATGG | GATCGCGGACCT |
| | CCGGGTAGGTAGATAGGTTTACTCTAAAGGGGGGG | GCTTCAGATG |
| | ACAGGATGACGACGATAAGTAGGG (SEQ ID NO: | (SEQ ID NO: 50) |
| | 191) | |
| | AGAATACAAGATCCCCCGAAGTCCCCCCCTTTAGA | TCTAAGTGGCCC |
| | GTAAACCTATCTACCTACCCGGCCATCTGGCAGGC | ATCACGGAC |
| | CCAACCAATTAACCAATTCTGATTAG (SEQ ID NO: | (SEQ ID NO: 50) |
| | 192) | |
| 42 | AAATACTGTCTAGTTACACCACCCTTCGAGAATGT | CTTCGTCTTCCAG |
| | CCCTGTAGGTAGATAGGGAAAGGGCCCTGGCGAG | TGGATCGA |
| | ACTAGGATGACGACGATAAGTAGGG (SEQ ID NO: | (SEQ ID NO: 51) |
| | 193) | |
| | TACTCATTGGCACTCCAGTCAGTCTCGCCAGGGCC | ATAAGAATACTT |
| | CTTTCCCTATCTACCTACAGGGACATTCTCGAAGGG | GCCTTGCAGGAT |
| | TAACCAATTAACCAATTCTGATTAG (SEQ ID NO: | C(SEQ ID NO: 51) |
| | 194) | |
| 43 | CACTACGCTCCTGACTTTGGCATCCGATGTCATGTT | ACTCGTATGTCC |
| | GAGGTAGGTAGATAGGATGAACCCGGGGCTGGGC | TCCAGTCG (SEQ |
| | TCAGGATGACGACGATAAGTAGGG (SEQ ID NO: | ID NO: 52) |
| | 195) | |
| | AAGGGTGCACTGATATGGACGAGCCCAGCCCCGGG | CAACCTGTCAAT |
| | TTCATCCTATCTACCTACCTCAACATGACATCGGAT | CTGTTCCACTAC |
| | GAACCAATTAACCAATTCTGATTAG (SEQ ID NO: | (SEQ ID NO: 52) |
| | 196) | |
| 44 | ATACTTGCCTTGCAGGATCTCAAAGAGGGAGATGG | ACTCTGATCTAC |
| | ACAGCTAGGTAGATAGGTCGGAAGGGTGCACTGAT | TGACCCGTACC |
| | ATAGGATGACGACGATAAGTAGGG (SEQ ID NO: | (SEQ ID NO: 53) |
| | 197) | |
| | ACCCGGGGCTGGGCTCGTCCATATCAGTGCACCCT | CCAACGACTATT |
| | TCCGACCTATCTACCTAGCTGTCCATCTCCCTCTTT | TGACTCGCCAC |
| | GAACCAATTAACCAATTCTGATTAG (SEQ ID NO: | (SEQ ID NO: 53) |
| | 198) | |
| 45 | ACACCTATAATGGTCTGTATTGACACCATTCTTTTA | TGGAAGCATTCT |
| | TTTAGGCCTTGTACGGGGTTGACCAGGATGACGAC | CTCTTCATCGTG |
| | GATAAGTAGGG (SEQ ID NO: 199) | (SEQ ID NO: 54) |
| | TGTAAATTTCCGCCCCTAGCGGTCAACCCCGTACA | CGAAGTTTGACG |
| | AGGCCTAAATAAAAGAATGGTGTCAAACCAATTAA | GCCTATACTGTA |
| | CCAATTCTGATTAG (SEQ ID NO: 200) | (SEQ ID NO: 54) |
| 46 | GCTAGGGGCGGAAATTTACAAAGCACACGAGTTAT | CTGAGCAGCGAG |
| | TGCCTGTTGAACTTATTTTCCCTTTAGGATGACGAC | AGCAGTTTC (SEQ |
| | GATAAGTAGGG (SEQ ID NO: 201) | ID NO: 55) |
| | CGGAGAGCGCACGCAGGTCAAAAGGGAAAATAAG | CCTCATTAGTCG |
| | TTCAACAGGCAATAACTCGTGTGCTTAACCAATTA | GGACTCGC |
| | ACCAATTCTGATTAG (SEQ ID NO: 202) | (SEQ ID NO: 55) |
| 47 | CCTAAAGACCGTCTGTTGCAACCATGCGTCCATGTT | GCGAGTCCCGAC |
| | GAACGGGGCAAATCCGGGTTTCACAGGATGACGAC | TAATGAGG |
| | GATAAGTAGGG (SEQ ID NO: 203) | (SEQ ID NO: 56) |
| | CCGAACCAGGCAACACAAGGGTGAAACCCGGATTT | GTCATTGCCACC |
| | GCCCCGTTCAACATGGACGCATGGTAACCAATTAA | CAGCTACT (SEQ |
| | CCAATTCTGATTAG (SEQ ID NO: 204) | ID NO: 56) |
| 48 | GGAAGACGATGGGGGAAATGTGGCATTACCTGAC | CAGTAGCTGGGT |
| | ACGGTTGTTCAGTCACATGTACGCTAAGGATGACG | GGCAATGAC |
| | ACGATAAGTAGGG (SEQ ID NO: 205) | (SEQ ID NO: 57) |
| | GGGGTTGGGTGGGGAGACCCTAGCGTACATGTGAC | GGTCACTGGGAT |
| | TGAACAACCGTGTCAGGTAATGCCAAACCAATTAA | CGTAGATTGTTT |
| | CCAATTCTGATTAG (SEQ ID NO: 206) | C (SEQ ID NO: 57) |
| 49 | ACAAAAATGGCGCAAGATGACAAGGTAAAGATCG | CAGTAGCTGGGT |
| | ACCTTTTGTAAAAACTATGACACGCCAGGATGACG | GGCAATGAC |
| | ACGATAAGTAGGG (SEQ ID NO: 207) | (SEQ ID NO: 58) |
| | TCTTACCCTAAGGAGAGAGTGGCGTGTCATAGTTT | GGTCACTGGGAT |
| | TTACAAAAGGTCGATCTTTACCTTGAACCAATTAA | CGTAGATTGTTT |
| | CCAATTCTGATTAG (SEQ ID NO: 208) | C (SEQ ID NO: 58) |
| 50 | ATGTCATTGTAAAAACTATGACACGCCACTCTCTCC | CACGAATCTGGT |
| | TTAGAGTGTTCGCAAGGGCGTCTGAGGATGACGAC | TGATTGTGAC |
| | GATAAGTAGGG (SEQ ID NO: 209) | (SEQ ID NO: 59) |
| | CTGGGAAGTTAACGCAGGCACAGACGCCCTTGCGA | CTGTTCCTTATGT |
| | ACACTCTAAGGAGAGAGTGGCGTGTAACCAATTAA | GCCTCCA |
| | CCAATTCTGATTAG (SEQ ID NO: 210) | (SEQ ID NO: 59) |
| K8 | GTCGACTATAACCTGGCGTGTAAACGTGTAACCCT | GGGAGAACCATG |
| | GCCAAACGGGAAACAGGTGTCTATCAGGATGACG | CCAGACTTTG |
| | ACGATAAGTAGGG (SEQ ID NO: 211) | (SEQ ID NO: 60) |
| | TTGAGTAACCAGCCGGCCAAGATAGACACCTGTTT | CATCGTGGAACG |
| | CCCGTTTGGCAGGGTTACACGTTTAAACCAATTAA | CACAGGTAA |
| | CCAATTCTGATTAG (SEQ ID NO: 212) | (SEQ ID NO: 60) |
| K8.1 | GGACCGAAGTTAATCCCTTAATCCTCTGGGATTAA | GCGTAAGAAACC |
| | TAACCTGGTGCTAGTAACCGTGTGCAGGATGACGA | CTACATAGTG |
| | CGATAAGTAGGG (SEQ ID NO: 213) | (SEQ ID NO: 61) |
| | TGTAGTGGTGGCAGAAAATGGCACACGGTTACTAG | GCATAGACTGGC |
| | CACCAGGTTATTAATCCCAGAGGATAACCAATTAA | ATGTGATT |
| | CCAATTCTGATTAG (SEQ ID NO: 214) | (SEQ ID NO: 61) |
| 52 | GTTTGGGGGTTGGGTTGTGGCGTGGTGGCTGGTCC | GTAGATGTACGT |
| | GCGGTGTCAGGTACGCGTAGATGTAAGGATGACGA | GTTGGTGATGCT |
| | CGATAAGTAGGG (SEQ ID NO: 215) | C (SEQ ID NO: 62) |
| | TTGTGAGCATCACCAACACGTACATCTACGCGTAC | GTCTGGCTTCATT |
| | CTGACACCGCGGACCAGCCACCACGAACCAATTAA | TGCTCTCGA |
| | CCAATTCTGATTAG (SEQ ID NO: 216) | (SEQ ID NO: 62) |
| 53 | TAGCTTTCGTCAGCGCTTGTGCGAGTAATCACATGC | TACTGGGACTAG |
| | CAGTTATGAACAACCGCCGAGGCTAGGATGACGAC | AACGCTCTG |
| | GATAAGTAGGG (SEQ ID NO: 217) | (SEQ ID NO: 63) |
| | ACGTTGGATAGACGGCTTGGAGCCTCGGCGGTTGT | GATAGGTTCGAC |
| | TCATAACTGGCATGTGATTACTCGCAACCAATTAA | ATAGGTTGGCT |
| | CCAATTCTGATTAG (SEQ ID NO: 218) | (SEQ ID NO: 63) |
| 54 | GGCCACAAATAAAGCCAGGGCCACCGTGGACGCT | GCTGTCGCCACT |
| | GTCATTAGCCGCCGCCAAATGCGGCCAGGATGACG | CGTACAAA |
| | ACGATAAGTAGGG (SEQ ID NO: 219) | (SEQ ID NO: 64) |
| | TCGAATCGCCCTAATAAACTGGCCGCATTTGGCGG | GTGCCAAGGTTC |
| | CGGCTAATGACAGCGTCCACGGTGGAACCAATTAA | GACTGGAC |
| | CCAATTCTGATTAG(SEQ ID NO: 220) | (SEQ ID NO: 64) |
| 55 | GCCTCGCGGCGGTATGTCGTCTCCATGGTACACCT | ACGTCACCCAGA |
| | GGACGTAGGTAGATAGGTGTTGCGGTATAAACCTT | CACACTCC |
| | TTAGGATGACGACGATAAGTAGGG (SEQ ID NO: | (SEQ ID NO: 65) |
| | 221) | |
| | AAGCGTGGTTGCCGCGTCCAAAAAGGTTTATACCG | GCTGTCGCCACT |
| | CAACACCTATCTACCTACGTCCAGGTGTACCATGG | CGTACAAA |
| | AGAACCAATTAACCAATTCTGATTAG (SEQ ID NO: | (SEQ ID NO: 65) |
| | 222) | |
| 56 | CACGTCCAGGTGTACCATGGAGACGACATACCGCC | ACGTCACCCAGA |
| | GCGAGTAGGTAGATAGGGCGCTGACAGTAAGGGTT | CACACTCC |
| | ATAGGATGACGACGATAAGTAGGG (SEQ ID NO: | (SEQ ID NO: 66) |
| | 223) | |
| | TGTCGCCACTCGTACAAAAAATAACCCTTACTGTC | GCTGTCGCCACT |
| | AGCGCCCTATCTACCTACTCGCGGCGGTATGTCGTC | CGTACAAA |
| | TAACCAATTAACCAATTCTGATTAG (SEQ ID NO: | (SEQ ID NO: 66) |
| | 224) | |
| 57 | AATATAAGAACCAAAGGACATGGTACAAGCAATG | CACCTTAAACAC |
| | ATAGACGGATTGCCAAACCCCATGGCAGGATGACG | AACACCAGACC |
| | ACGATAAGTAGGG (SEQ ID NO: 225) | (SEQ ID NO: 67) |
| | TGGAATACGGGAGACACTCTGCCATGGGGTTTGGC | CCAGGCAATTCT |
| | AATCCGTCTATCATTGCTTGTACCAAACCAATTAAC | GCGGCTAG |
| | CAATTCTGATTAG (SEQ ID NO: 226) | (SEQ ID NO: 67) |
| K9 | CTCCCTCCCATAACAATACGGTGTAGGCATTTTGTA | TGAATGGTAACT |
| | TTATTGTCCCGCAACCAGACTAGCAGGATGACGAC | GTCTGGACAC |
| | GATAAGTAGGG (SEQ ID NO: 227) | (SEQ ID NO: 68) |
| | CACTGGACATTGCGGCGCGAGCTAGTCTGGTTGCG | GAGAACAAAGCT |
| | GGACAATAATACAAAATGCCTACACAACCAATTAA | ACGAGGAGG |
| | CCAATTCTGATTAG (SEQ ID NO: 228) | (SEQ ID NO: 68) |
| K10 | ACTACAAGATTACATCCGGTTTTATAATTCACATAT | CTCTTGACCTGG |
| | ATGAACCTGAGGTAGATGCGCCCTAGGATGACGAC | TAACCCTGG |
| | GATAAGTAGGG (SEQ ID NO: 229) | (SEQ ID NO: 69) |
| | CGTGTGGATACCAGTGAATGAGGGCGCATCTACCT | CAGTTGATGATG |
| | CAGGTTCATATATGTGAATTATAAAAACCAATTAA | CCAATGCCG |
| | CCAATTCTGATTAG (SEQ ID NO: 230) | (SEQ ID NO: 69) |
| K10.5 | CCACAGCCCGTCAAACCACAGGGACCCTGTTGGCT | GTCGCCACGCCC |
| | GACTACAATGCACATGCAGATTCTTAGGATGACGA | ACAACATC |
| | CGATAAGTAGGG (SEQ ID NO: 231) | (SEQ ID NO: 70) |
| | TGACCTCACACTGCTTGATAAAGAATCTGCATGTG | CCCGTTGGCAAA |
| | CATTGTAGTCAGCCAACAGGGTCCCAACCAATTAA | CATAGATCCGTC |
| | CCAATTCTGATTAG (SEQ ID NO: 232) | (SEQ ID NO: 70) |
| K11 | GGTGGGGGCTCAGGGTTTTGTAGGGAGGGATATGC | GCACTGTCCACC |
| | ACAGTTAGGTAGATAGGTTGTTTTTTGAAGAGCCA | CTCTAATACAAG |
| | GAAGGATGACGACGATAAGTAGGG (SEQ ID NO: | (SEQ ID NO: 71) |
| | 233) | |
| | ATGACCCAAACCCCGACGGTTCTGGCTCTTCAAAA | CTCACACCAGTT |
| | AACAACCTATCTACCTAACTGTGCATATCCCTCCCT | GGTCCCTTTG |
| | AAACCAATTAACCAATTCTGATTAG (SEQ ID NO: | (SEQ ID NO: 71) |
| | 234) | |
| 58 | TCATGGTCAACAAACCAAGAAAAACACATGTATTA | GGTAAAGAGTGT |
| | TTCAAGGTGTCAAATCAGGGGGTTAAGGATGACGA | GAACGAGTACAG |
| | CGATAAGTAGGG (SEQ ID NO: 235) | G |
| | | (SEQ ID NO: 72) |
| | AAGGTGCCCAAAACCACATTTAACCCCCTGATTTG | GTGTGACTGACG |
| | ACACCTTGAATAATACATGTGTTTTAACCAATTAAC | ATTTGTGAAGGT |
| | CAATTCTGATTAG (SEQ ID NO: 236) | (SEQ ID NO: 72) |
| 59 | GAGCGACAGAGCGCGCTCACTGTCCAGGCGGCACA | GCCGTAGACGCA |
| | TGGTGGATTGCGGCCGTAGACGCACAGGATGACGA | CAGAGAAATC |
| | CGATAAGTAGGG (SEQ ID NO: 237) | (SEQ ID NO: 73) |
| | AGCTTTCCTGTGATTTCTCTGTGCGTCTACGGCCGC | CTTCAGTGCCTG |
| | AATCCACCATGTGCCGCCTGGACAAACCAATTAAC | GCAGATCC |
| | CAATTCTGATTAG (SEQ ID NO: 238) | (SEQ ID NO: 73) |
| 60 | TTAGGGGAGGTGGAAGTGTGCGACATGGACAGGTT | CGTGGGAAACAT |
| | AACCTTGGCCTCACCCGGCTTGCAGAGGATGACGA | CAAGGTGC |
| | CGATAAGTAGGG (SEQ ID NO: 239) | (SEQ ID NO: 74) |
| | GTCTGTCAGTAGGTAGGTCTCTGCAAGCCGGGTGA | GCACAGTTCCCT |
| | GGCCAAGGTTAACCTGTCCATGTCGAACCAATTAA | TTGATTCTCATC |
| | CCAATTCTGATTAG (SEQ ID NO: 240) | (SEQ ID NO: 74) |
| 61 | AACTGAATCCATTGGCCTCACCCGGCTTGCAGAGA | GTCTATGAGAGA |
| | CCTACGACCTTACAGAAACACAGTCAGGATGACGA | TTGGGCACAC |
| | CGATAAGTAGGG (SEQ ID NO: 241) | (SEQ ID NO: 75) |
| | GAGGCCGCGTGTGGCCCCTGGACTGTGTTTCTGTA | GCTCTGTTGTGCT |
| | AGGTCGTAGGTCTCTGCAAGCCGGGAACCAATTAA | GCTGTTTA |
| | CCAATTCTGATTAG (SEQ ID NO: 242) | (SEQ ID NO: 75) |
| 62 | TCCCAAGTGAACCTGACAAAATGTCCGGACAGACA | GTGGACGCCGCA |
| | TGACCATCCACGCCGGCAATGGACGAGGATGACGA | TATTTAGAGAG |
| | CGATAAGTAGGG (SEQ ID NO: 243) | (SEQ ID NO: 76) |
| | CTTTTCAAGAGCGTCTGTGCCGTCCATTGCCGGCGT | GGTACATGACGC |
| | GGATGGTCATGTCTGTCCGGACATAACCAATTAAC | AGTTGCTGA |
| | CAATTCTGATTAG (SEQ ID NO: 244) | (SEQ ID NO: 76) |
| 63 | AGCCGCATTTTCAGCCTGCACCTTCATATCCACGCC | GAAACGTACTCC |
| | GGCAAGGCCATGGCAGCCCAGCCTAGGATGACGA | CGGTCTGC |
| | CGATAAGTAGGG (SEQ ID NO: 245) | (SEQ ID NO: 77) |
| | GCCATTCCCTCCATGTACAGAGGCTGGGCTGCCAT | GTATAACCACCC |
| | GGCCTTGCCGGCGTGGATATGAAGGAACCAATTAA | TGTCCTCTGGT |
| | CCAATTCTGATTAG (SEQ ID NO: 246) | (SEQ ID NO: 77) |
| 64 | CGCTGGCAGGCCTCCGGAAACTGTTTGTCGAATAG | GGTCACCCATAG |
| | AGGCCCTCCACGGTTGTCCAATCGTAGGATGACGA | TACCCATCAG |
| | CGATAAGTAGGG (SEQ ID NO: 247) | (SEQ ID NO: 78) |
| | CTGGCAAAAAGAAATAGGCAACGATTGGACAACC | CTGCGAGGCTGC |
| | GTGGAGGGCCTCTATTCGACAAACAGAACCAATTA | CCTATTAA |
| | ACCAATTCTGATTAG (SEQ ID NO: 248) | (SEQ ID NO: 78) |
| 65 | AGAAGTGGTACTTGTGACTCCACGGTTGTCCAATC | GTCACAGCGGTA |
| | GTTGCCTTCCACACAGGCGGGCGAAAGGATGACGA | TATTGGGC |
| | CGATAAGTAGGG (SEQ ID NO: 249) | (SEQ ID NO: 79) |
| | AGTGTTCCTCCTGAGGCTATTTCGCCCGCCTGTGTG | AAAGCCACATAT |
| | GAAGGCAACGATTGGACAACCGTGAACCAATTAAC | TCCTCCACTG |
| | CAATTCTGATTAG (SEQ ID NO: 250) | (SEQ ID NO: 79) |
| 66 | TAGGCCGTGCGGTCGCGCTGGTGAGAAGGTCATGG | GTTGGAGAGCAA |
| | CCCTGTAGGTAGATAGGGATCAGCGCTGGGATCGC | GGTGGACACG |
| | TTAGGATGACGACGATAAGTAGGG (SEQ ID NO: | (SEQ ID NO: 80) |
| | 251) | |
| | AACCAAACCAAGACACAAGAAAGCGATCCCAGCG | ACCGTGCTGCAT |
| | CTGATCCCTATCTACCTACAGGGCCATGACCTTCTC | TCTAACCGTAC |
| | ACAACCAATTAACCAATTCTGATTAG (SEQ ID NO: | (SEQ ID NO: 80) |
| | 252) | |
| 67 | GGGGCCTTGCCAGCCCCACCCCGCTGTCGCCATGA | GAGAGTTGGAAG |
| | GTGTCTAGGTAGATAGGGTTGGTAAGCGTGTAGTG | AGACGCGGG |
| | GAAGGATGACGACGATAAGTAGGG (SEQ ID NO: | (SEQ ID NO: 81) |
| | 253) | |
| | ATACCACCCGACACAGTTCGTCCACTACACGCTTA | CCACCTTGCTCTC |
| | CCAACCCTATCTACCTAGACACTCATGGCGACAGC | CAACACCAG |
| | GGAACCAATTAACCAATTCTGATTAG (SEQ ID NO: | (SEQ ID NO: 81) |
| | 254) | |
| 67.5 | GTCTGACCAGCTTCTGCCTCGTGACATGCAAATTTT | CCACCTTGCTCTC |
| | ATTTTAGGTAGATAGGCCCACGATCTATTGTAGATT | CAACACCAG |
| | AGGATGACGACGATAAGTAGGG (SEQ ID NO: 255) | (SEQ ID NO: 82) |
| | GACAGTAGTTGATGGCGTTCAATCTACAATAGATC | GAGAGTTGGAAG |
| | GTGGGCCTATCTACCTAAAATAAAATTTGCATGTC | AGACGCGGG |
| | ACAACCAATTAACCAATTCTGATTAG (SEQ ID NO: | (SEQ ID NO: 82) |
| | 256) | |
| 68 | TCTACAATAGATCGTGGGAAATAAAATTTGCATGT | TTATTCGGGAGC |
| | CACGATAGGTAGATAGGGGCAGAAGCTGGTCAGA | TAACCGCAC |
| | CGCAGGATGACGACGATAAGTAGGG (SEQ ID NO: | (SEQ ID NO: 83) |
| | 257) | |
| | TGGAACCCAACATGGAGTACGCGTCTGACCAGCTT | CACCTTTCATGG |
| | CTGCCCCTATCTACCTATCGTGACATGCAAATTTTA | CAGTACATTGC |
| | TAACCAATTAACCAATTCTGATTAG (SEQ ID NO: | (SEQ ID NO: 83) |
| | 258) | |
| 69 | ACGCTTGAGCTGGTCCCGGGCCTTCGCACCCCATC | TTATTCGGGAGC |
| | CACCGCCTCACATGTAGCCTGTCACAGGATGACGA | TAACCGCAC |
| | CGATAAGTAGGG (SEQ ID NO: 259) | (SEQ ID NO: 84) |
| | CAGTTGCAATAGGAGCTGGGGTGACAGGCTACATG | CACCTTTCATGG |
| | TGAGGCGGTGGATGGGGTGCGAAGGAACCAATTA | CAGTACATTGC |
| | ACCAATTCTGATTAG (SEQ ID NO: 260) | (SEQ ID NO: 84) |
| K12 | ATTTTATTTTACTGACACTCTTTGGGAGGGCACGCT | TCGCCTTCAAAC |
| | AGCTGCATTGGGATTGGAGTGAGGAGGATGACGAC | AGAAGCACG |
| | GATAAGTAGGG (SEQ ID NO: 261) | (SEQ ID NO: 85) |
| | AACCTGGTGCCCTCCTCCCTCCTCACTCCAATCCCA | GATGTTTCCGTTC |
| | ATGCAGCTAGCGTGCCCTCCCAAAAACCAATTAAC | TACAGGCGG |
| | CAATTCTGATTAG (SEQ ID NO: 262) | (SEQ ID NO: 85) |
| K13 | CATACATTCTACGGACCAAAAATTAGCAACAGCTT | TGTCATCCGTGC |
| | GTTATGGTGCCGGCTTGTATATGTGAGGATGACGA | CCAGTTTC |
| | CGATAAGTAGGG (SEQ ID NO: 263) | (SEQ ID NO: 86) |
| | TTTTTCCACATCGGTGCCTTCACATATACAAGCCGG | GTTCTCACGACC |
| | CACCATAACAAGCTGTTGCTAATTAACCAATTAAC | CATCTACCTC |
| | CAATTCTGATTAG (SEQ ID NO: 264) | (SEQ ID NO: 86) |
| 72 | AAGGAAAATTTATTTTTCCGCCCTAAACAAAATCA | ATGGGTCGTGAG |
| | CAAGCATAGAGTGGCGAGCGTATGTAGGATGACG | AACACTGC |
| | ACGATAAGTAGGG (SEQ ID NO: 265) | (SEQ ID NO: 87) |
| | CCAGGCTCTAGAGGTAGGCCACATACGCTCGCCAC | CTGCGATCTCCA |
| | TCTATGCTTGTGATTTTGTTTAGGGAACCAATTAAC | TCCTGTGG |
| | CAATTCTGATTAG (SEQ ID NO: 266) | (SEQ ID NO: 87) |
| 73 | GGTGGCTTCTAGGGAGGAAAAAGGGGGAGAGGTG | AAACTGAAGAAG |
| | TGGCTTCCTCGGGAAATCTGGTCTGAAGGATGACG | GCGTGTCTGC |
| | ACGATAAGTAGGG (SEQ ID NO: 267) | (SEQ ID NO: 88) |
| | CCATAATTTTACTTTGGTTGTCAGACCAGATTTCCC | GAAGTGACTGCC |
| | GAGGAAGCCACACCTCTCCCCCTTAACCAATTAAC | AAACCACAC |
| | CAATTCTGATTAG (SEQ ID NO: 268) | (SEQ ID NO: 88) |
| K14 | TGCTCCCCCGTGGACGACGCCGAGTGCCTCTCGGG | TGTGTTGAAGGA |
| | GGTCCCTAGATGGACACCCCGTGAAAGGATGACGA | CGGATCAGG |
| | CGATAAGTAGGG (SEQ ID NO: 269) | (SEQ ID NO: 89) |
| | GGGGTGGGTAAGCACGACGGTTCACGGGGTGTCCA | CAAGAAGATCAA |
| | TCTAGGGACCCCCGAGAGGCACTCGAACCAATTAA | CGACCACCACTA |
| | CCAATTCTGATTAG (SEQ ID NO: 270) | (SEQ ID NO: 89) |
| 74 | CGTGGCTAAACAACACCTATACTACTTGTTATTGTA | TGTGTTGAAGGA |
| | GGCCCCCGCGGATGTCTACGTGCCAGGATGACGAC | CGGATCAGG |
| | GATAAGTAGGG (SEQ ID NO: 271) | (SEQ ID NO: 90) |
| | AGATTAAATTAAGGGGGAAGGGCACGTAGACATC | CAAGAAGATCAA |
| | CGCGGGGGCCTACAATAACAAGTAGTAACCAATTA | CGACCACCACTA |
| | ACCAATTCTGATTAG (SEQ ID NO: 272) | (SEQ ID NO: 90) |
| 75 | TTATGCGATTAAATGAGGGGTCTGATCCCAAAAGC | TCTAGCCTCCCG |
| | AATGTGCCTAGAGGGTGCCCCGCCCAGGATGACGA | TTCCCATG (SEQ |
| | CGATAAGTAGGG (SEQ ID NO: 273) | ID NO: 91) |
| | ACTACAGAGGGTGTCCCCGGGGGCGGGGCACCCTC | AAAGCCCTAACC |
| | TAGGCACATTGCTTTTGGGATCAGAAACCAATTAA | CAAGTCTGACTA |
| | CCAATTCTGATTAG (SEQ ID NO: 274) | C (SEQ ID NO: 91) |
| K15 | ACAACAACTCTATTGTAAGCCCTGTGGATACCTAG | GAGCCTTGTGTC |
| | TCAAACCCTCCACGACCACAGACTTAGGATGACGA | GGGAATACTTAG |
| | CGATAAGTAGGG (SEQ ID NO: 275) | (SEQ ID NO: 92) |
| | AAAAAGGTATCGATGTCAAAAAGTCTGTGGTCGTG | TATTACGCAGGC |
| | GAGGGTTTGACTAGGTATCCACAGGAACCAATTAA | ACAGGTTGCTC |
| | CCAATTCTGATTAG (SEQ ID NO: 276) | (SEQ ID NO: 92) |

TABLE S2
Primers for the construction of rescued
KSHV mutants. The primers are listed according
to the steps in construction of the universal
transfer construct (UTC). Step 1 inserted the
ORF into pUC19. Step 2 inserted the 50 bp
sequence duplication and the kanamycin
resistance cassette into the unique
restriction enzyme site located inside
the ORF. Step 3 was used to create the
linear UTC for electroporation.
Rescued Primers

| | STEP 1 | STEP 2 | STEP 3 |
|---|---|---|---|
| ORF | (5′→3′) | (5′→3′) | (5′→3′) |
| R59 | GAGTCCAAGC | GAGTCCACGC | TCGTCTCCAG |
| | TTATGTCGCA | GTGAGCTATT | AACACCCAG |
| | CACTTCCACC | CGGTGCGAAT | (SEQ ID |
| | (SEQ ID | GTACTCGACG | NO: 281) |
| | NO: 277) | CTGGCATAGC | |
| | | CTTTTATCGA | |
| | | AAAGGATGAC | |
| | | GACGATAAGT | |
| | | AGGG | |
| | | (SEQ ID | |
| | | NO: 279) | |
| | GAGTCCGAAT | GAGTCCACGC | CAGATAACTG |
| | TCAACGAGTA | GTAACCAATT | AAGAGCGACA |
| | CAGGGCCTTG | AACCAATTCT | GAG |
| | (SEQ ID | GATTAG | (SEQ ID |
| | NO: 278) | (SEQ ID | NO: 282) |
| | | NO: 280) | |
| R62 | GAGTCCGCAT | GAGTCCCCAT | TTGTCTGGTG |
| | GCTTCCATCA | GGTCCAGCGC | AAGGCTCC |
| | ACAGCTTTGT | CGGGAACCGG | (SEQ ID |
| | CTGG | CAGGCCTAAA | NO: 287) |
| | (SEQ ID | CGTTACTATT | |
| | NO: 283) | TATGCCTCGT | |
| | | TGAGGATGAC | |
| | | GACGATAAGT | |
| | | AGGG | |
| | | (SEQ ID | |
| | | NO: 285) | |
| | GAGTCCGAAT | GAGTCCCCAT | GGGACAGCTC |
| | TCTGAGAGAT | GGAACCAATT | CCAAGTGAA |
| | TGGGCACACA | AACCAATTCT | (SEQ ID |
| | TA | GATTAG | NO: 288) |
| | (SEQ ID | (SEQ ID | |
| | NO: 284) | NO: 286) | |

TABLE S3
KSHV ORFs homologous to HSV, VZV, EBV, HCMV, and MHV-68 ORFs categorized by
growth properties of their respective inactivation mutants in cultured cells.

| Gene # | KSHV | HSV¹¹⁶ | VZV^{53, 117} | HCMV^{3, 4, 118} | EBV⁵² | MHV-68^{6, 7, 66} |
|---|---|---|---|---|---|---|
| 1 | K1 | | | | | |
| 2 | ORF4 | | | | | ORF4 |
| 3 | ORF6 | UL29 | 29 | UL57 | BALF2¹¹⁹ | ORF6 |
| 4 | ORF7 | UL28 | 30 | UL56 | ^{#}BALF3¹²⁰ | ORF7 |
| 5 | ORF8 | UL27 | 31 | UL55 | BALF4¹²¹ | ORF8 |
| 6 | ORF9 | UL30 | 28 | UL54 | BALF5¹¹⁹ | ORF9 |
| 7 | 10.1 | | | | | |
| 8 | ORF10 | | | UL82 UL83 | | ORF10 |
| | | | | UL84 | | |
| 9 | 11AA | | | | | |
| 10 | ORF11 | | | UL82 UL83 | Raji LF2 | ORF11 |
| | | | | UL84 | | |
| 11 | K2 | | | | | |
| 12 | ORF2 | | | | | |
| 13 | K3 | | | | | K3 |
| 14 | ORF70 | | | | | |
| 15 | K4 | | | | | |
| 16 | K4.1 | | | | | |
| 17 | K4.2 | | | | | |
| 18 | K5 | | | | | |
| 19 | K6 | | | | | |
| 20 | K7 | | | | | |
| 21 | ORF16 | | | | BHRF1¹²² | |
| 22 | ORF17 | UL26 | 33 | UL80 | ^{#}BVRF2^{123, 124} | ORF17 |
| 23 | ORF17.5 | UL26.5 | 33.5 | UL80.5 | BdRF1¹²⁴ | |
| 24 | ORF18 | | | UL79 | | ORF18 |
| 25 | ORF19 | UL25 | 34 | UL77 | BVRF1 | ORF19 |
| 26 | ORF20 | UL24 | 35 | UL76 | BXRF1 | ORF20 |
| 27 | ORF21 | UL23 | | | BXLF1 | ORF21 |
| 28 | ORF22 | UL22 | 37 | UL75 | BXLF2¹²⁵ | ORF22 |
| 29 | ORF23 | UL21 | 38 | UL88 | BTRF1 | ORF23 |
| 30 | ORF24 | | | UL87 | ‡BcRF1^{126, 127} | ORF24 |
| 31 | ORF25 | UL19 | 40 | UL86 | BcLF1¹²⁴ | ORF25* |
| 32 | ORF26 | UL18 | 41 | UL85 | BDLF1¹²⁴ | ORF26 |
| 33 | ORF27 | | | | BDLF2 | ORF27 |
| 34 | ORF28 | | | | BDLF3¹²⁸ | |
| 35 | ORF29B | UL15 | 42/45 | UL89.2 | BDRF1¹²⁹ | ORF29B |
| 36 | 30.1 | | | | | |
| 37 | ORF30 | | | UL91 | BDLF3.5 | ORF30 |
| 38 | ORF31 | | | UL92 | BDLF4¹³⁰ | ORF31 |
| 39 | ORF32 | UL17 | 43 | UL93 | BGLF1 | ORF32 |
| 40 | ORF33 | UL16 | 44 | UL94 | BGLF2¹³¹ | ORF33 |
| 41 | ORF29A | UL15 | 42/45 | UL89.1 | BGRF1¹²⁹ | ORF29A |
| 42 | 34.1 | | | | | |
| 42 | ORF34 | UL14 | 46 | UL95 | BGLF3 | ORF34 |
| 43 | ORF35 | | | | BGLF3.5¹³² | ORF35 |
| 44 | ORF36 | UL13 | 47 | UL97 | BGLF4^{132, 133} | ORF36 |
| 45 | ORF37 | UL12 | 48 | UL98 | BGLF5¹³⁴ | ORF37* |
| 46 | ORF38 | UL11 | 49 | UL99 | BBLF1 | ORF38 |
| 47 | ORF39 | UL10 | 50 | UL100 | BBRF3 | ORF39* |
| 48 | ORF40 | UL9¹³⁵ | 51 | UL102 | BBLF2¹¹⁹ | ORF40 |
| 49 | ORF41 | UL8 | 52 | UL102 | BBLF3¹¹⁹ | |
| 50 | ORF42 | UL7 | 53 | UL103 | BBRF2¹³⁶ | ORF42 |
| 51 | ORF43 | UL6 | 54 | UL104 | BBRF1¹³⁷ | ORF43 |
| 52 | ORF44 | UL5 | 55 | UL105 | BBLF4¹¹⁹ | ORF44 |
| 53 | ORF45 | UL3¹³⁸ | | | BKRF4¹³⁹ | ORF45 |
| 54 | ORF46 | UL2 | 59 | UL114 | ‡BKRF3^{140, 141} | ORF46 |
| 55 | ORF47 | UL1 | 60 | UL115 | BRKF2 | ORF47 |
| 56 | ORF48 | | | | BRRF2¹⁴² | ORF48 |
| 57 | ORF49 | | | | BRRF1¹⁴³ | ORF49 |
| 58 | ORF50 | | | | BRLF1¹⁴⁴ | ORF50 |
| 59 | K8 | | | | | |
| 60 | K8.1 | | | | | |
| 61 | ORF52 | | | | BLRF2 | ORF52 |
| 62 | ORF53 | UL49A | 9A | UL73 | BLRF1¹⁴⁵ | ORF53 |
| 63 | ORF54 | UL50 | 8 | UL72 | BLLF3 | ORF54 |
| 64 | ORF55 | UL51 | 7 | UL71 | BSRF1¹⁴⁶ | ORF55 |
| 65 | ORF56 | UL52 | 6 | UL70 | BSLF1¹¹⁹ | ORF56* |
| 66 | ORF57 | UL54 | 4 | UL69 | BSLF2/BMLF1¹⁴⁷ | ORF57 |
| 67 | K9 | | | | | |
| 68 | K10 | | | | | |
| 69 | K10.5 | | | | | |
| 70 | K11 | | | | | |
| 71 | ORF58 | UL43 | | | BMRF2 | ORF58 |
| 72 | ORF59 | UL42 | 16 | UL44 | BMRF1¹¹⁹ | ORF59 |
| 73 | ORF60 | UL40 | 18 | | BaRF1¹⁴⁸ | ORF60 |
| 74 | ORF61 | UL39 | 19 | UL45 | BORF2¹⁴⁹ | ORF61* |
| 75 | ORF62 | UL38 | 20 | UL46 | BORF1¹²⁴ | ORF62 |
| 76 | ORF63 | UL37 | 21 | UL47 | BOLF1¹⁵⁰ | ORF63 |
| 77 | ORF64 | UL36 | 22 | UL48 | BPLF1¹⁵¹ | ORF64 |
| 78 | ORF65 | UL35 | 23 | UL48A | BFRF3¹²⁴ | M9/ORF65* |
| 79 | ORF66 | | 24 | UL49 | BFRF2¹⁵² | ORF66* |
| 80 | ORF67 | UL34 | 25 | UL50 | BFRF1¹⁵³ | ORF67 |
| 81 | ORF67.5 | UL33 | 25 | UL51 | †BFRF1A¹²⁹ | |
| 82 | ORF68 | UL32 | 26 | UL52 | †FLF1¹²⁹ | ORF68 |
| 83 | ORF69 | UL31 | 27 | UL53 | BFLF2¹⁵⁴ | ORF69 |
| 84 | K12 | | | | | |
| 85 | K13/ORF71 | | | | | |
| 86 | ORF72 | | | | | ORF72 |
| 87 | ORF73 | | | | | ORF73 |
| 88 | K14 | | | | | |
| 89 | ORF74 | | | | | ORF74 |
| 90 | ORF75 | | | | BNRF1 | ORF75A |
| | | | | | | ORF75B |
| | | | | | | ORF75C |
| 91 | K15 | | | | LMP2A | |

ORFs in which gene-inactivation mutants failed to grow are marked in red and ORFs in which gene-inactivation mutants were attenuated in growth compared to parental viruses are marked in orange.
*Marks the ORFs where the classification assignment between two independent studies disagreed, and the asterisk color indicates the alternative classification^{3, 4, 6, 7, 53, 66}. Italics indicate ORFs that are positional homologs to KSHV ORF's.
^{†}Indicates that the ORF's essentiality was assessed as a double mutant.
^{#}Indicates essentiality was inferred from knockdown studies.
^{‡}Indicates two or more studies disagree on essentiality.

TABLE S4
KSHV lytic antigen expression in BAC16-infected iSLK cells.
Human iSLK cells were infected with BAC16 (MOI = 1). For
“Pre-induced reactivation” sample, cells were incubated in
normal/uninduced conditions in the absence of doxycycline and
sodium butyrate and harvested at 2 dpi. For “Induced reactivation”
samples, cells were incubated in normal/uninduced conditions for
2 days, then induced in the presence of doxycycline and sodium
butyrate at 2 dpi, and harvested at 5 dpi. For “Spontaneous reactivation”
samples, cells were incubated in normal/uninduced conditions and
harvested at 6 dpi. The harvested cells were fixed, stained for lytic
antigens, and analyzed by flow cytometry. The values are the average
from three independent experiments. Each experiment was performed
in triplicate. Experimental details are described in Methods.

| | Gene expression | | |
|---|---|---|---|
| Conditions | ORF45 | K8 | K8.1 |
| Pre-induced | 1.24% | 0.61% | 0.33% |
| Reactivation | | | |
| Induced | 34.03% | 26.68% | 5.44% |
| Reactivation | | | |
| Spontaneous | | | |
| Reactivation | 0.31% | 0.25% | 0.09% |

### REFERENCES

1 Blossom A. Damania, E. C. Fields Virology 6th edn, Vol. 2 Chapter 65 (Lippincott and Williams, 2013).
2 Philip E. Pellett, B. R. Feilds Virology 6th edn, Vol. 2 Chapter 59 (Lippincott Wilkins 2013).
3 Dunn, W. et al. Functional profiling of a human cytomegalovirus genome. Proc Natl Acad Sci USA 100, 14223-14228, doi:10.1073/pnas.2334032100 (2003).
4 Yu, D., Silva, M. C. & Shenk, T. Functional map of human cytomegalovirus AD169 defined by global mutational analysis. Proc Natl Acad Sci USA 100, 12396-12401, doi:10.1073/pnas.1635160100 (2003).
5 Zhang, Z. et al. Genome-wide mutagenesis reveals that ORF7 is a novel VZV skin-tropic factor. PLoS Pathog 6, e1000971, doi:10.1371/journal.ppat.1000971 (2010).
6 Song, M. J. et al. Identification of viral genes essential for replication of murine gamma-herpesvirus 68 using signature-tagged mutagenesis. Proc Natl Acad Sci USA 102, 3805-3810, doi:10.1073/pnas.0404521102 (2005).
7 Moorman, N. J., Lin, C. Y. & Speck, S. H. Identification of candidate gammaherpesvirus 68 genes required for virus replication by signature-tagged transposon mutagenesis. J Virol 78, 10282-10290, doi:10.1128/JVI.78.19.10282-10290.2004 (2004).
8 Russo, J. J. et al. Nucleotide sequence of the Kaposi sarcoma-associated herpesvirus (HHV8). Proc Natl Acad Sci USA 93, 14862-14867, doi:10.1073/pnas.93.25.14862 (1996).
9 Arias, C. et al. KSHV 2.0: a comprehensive annotation of the Kaposi's sarcoma-associated herpesvirus genome using next-generation sequencing reveals novel genomic and functional features. PLoS Pathog 10, e1003847, doi:10.1371/journal.ppat.1003847 (2014).
10 Cai, X. et al. Kaposi's sarcoma-associated herpesvirus expresses an array of viral microRNAs in latently infected cells. Proc Natl Acad Sci USA 102, 5570-5575, doi:10.1073/pnas.0408192102 (2005).
11 Pfeffer, S. et al. Identification of microRNAs of the herpesvirus family. Nat Methods 2, 269-276, doi:10.1038/nmeth746 (2005).
12 Tagawa, T. et al. Discovery of Kaposi's sarcoma herpesvirus-encoded circular RNAs and a human antiviral circular RNA. Proc Nat Acad Sci USA 115, 12805-12810, doi:10.1073/pnas.1816183115 (2018).
13 Abere, B. et al. Kaposi's Sarcoma-Associated Herpesvirus-Encoded circRNAs Are Expressed in Infected Tumor Tissues and Are Incorporated into Virions. mBio 11, doi:10.1128/mBio.03027-19 (2020).
14 Schifano, J. M., Corcoran, K., Kelkar, H. & Dittmer, D. P. Expression of the Antisense-to-Latency Transcript Long Noncoding RNA in Kaposi's Sarcoma-Associated Herpesvirus. J Virol 91, doi:10.1128/JVI.01698-16 (2017).
15 Walker, L. R., Hussein, H. A. M. & Akula, S. M. Disintegrin-like domain of glycoprotein B regulates Kaposi's sarcoma-associated herpesvirus infection of cells. J Gen Virol 95, 1770-1782, doi:10.1099/vir.0.066829-0 (2014).
16 Luna, R. E. et al. Kaposi's sarcoma-associated herpesvirus glycoprotein K8.1 is dispensable for virus entry. J Virol 78, 6389-6398, doi:10.1128/jvi.78.12.6389-6398.2004 (2004).
17 Ye, F. C. et al. Disruption of Kaposi's sarcoma-associated herpesvirus latent nuclear antigen leads to abortive episome persistence. J Virol 78, 11121-11129, doi:10.1128/jvi.78.20.11121-11129.2004 (2004).
18 Nishimura, M., Watanabe, T., Yagi, S., Yamanaka, T. & Fujimuro, M. Kaposi's sarcoma-associated herpesvirus ORF34 is essential for late gene expression and virus production. Sci Rep 7, 329, doi:10.1038/s41598-017-00401-7 (2017).
19 Xu, Y. et al. A Kaposi's sarcoma-associated herpesvirus/human herpesvirus 8 ORF50 deletion mutant is defective for reactivation of latent virus and DNA replication. J Virol 79, 3479-3487, doi:10.1128/jvi.79.6.3479-3487.2005 (2005).
20 Zhu, F. X., Li, X., Zhou, F., Gao, S. J. & Yuan, Y. Functional characterization of Kaposi's sarcoma-associated herpesvirus ORF45 by bacterial artificial chromosome-based mutagenesis. J Virol 80, 12187-12196, doi:10.1128/JVI.01275-06 (2006).
21 Majerciak, V., Pripuzova, N., McCoy, J. P., Gao, S. J. & Zheng, Z. M. Targeted disruption of Kaposi's sarcoma-associated herpesvirus ORF57 in the viral genome is detrimental for the expression of ORF59, K8alpha, and K8.1 and the production of infectious virus. J Virol 81, 1062-1071, doi:10.1128/JVI.01558-06 (2007).
22 Han, Z. & Swaminathan, S. Kaposi's sarcoma-associated herpesvirus lytic gene ORF57 is essential for infectious virion production. J Virol 80, 5251-5260, doi:10.1128/jvi.02570-05 (2006).
23 Bergson, S. et al. The Kaposi's-sarcoma-associated herpesvirus or35 gene product is required for efficient lytic virus reactivation. Virology 499, 91-98, doi:10.1016/j.virol.2016.09.008 (2016).
24 Peng, C., Chen, J., Tang, W., Liu, C. & Chen, X. Kaposi's sarcoma-associated herpesvirus ORF6 gene is essential in viral lytic replication. PLoS One 9, e99542, doi:10.1371/journal.pone.0099542 (2014).
25 Bala, K. et al. Kaposi's sarcoma herpesvirus K15 protein contributes to virus-induced angiogenesis by recruiting PLCyl and activating NFAT1-dependent RCAN1 expression. PLoS Pathog 8, e1002927, doi:10.1371/journal.ppat.1002927 (2012).
26 Full, F. et al. Kaposi's sarcoma associated herpesvirus tegument protein ORF75 is essential for viral lytic replication and plays a critical role in the antagonization of ND10-instituted intrinsic immunity. PLoS Pathog 10, e1003863, doi:10.1371/journal.ppat.1003863 (2014).
27 Zhang, Z. et al. The KI Protein of Kaposi's Sarcoma-Associated Herpesvirus Augments Viral Lytic Replication. J Virol 90, 7657-7666, doi:10.1128/jvi.03102-15 (2016).
28 Wang, Y., Sathish, N., Hollow, C. & Yuan, Y. Functional characterization of Kaposi's sarcoma-associated herpesvirus open reading frame K8 by bacterial artificial chromosome-based mutagenesis. J Virol 85, 1943-1957, doi:10.1128/JVI.02060-10 (2011).
29 McDowell, M. E., Purushothaman, P., Rossetto, C. C., Pari, G. S. & Verma, S. C. Phosphorylation of Kaposi's sarcoma-associated herpesvirus processivity factor ORF59 by a viral kinase modulates its ability to associate with RTA and oriLyt. J Virol 87, 8038-8052, doi:10.1128/JVI.03460-12 (2013).
30 Brulois, K. et al. Association of Kaposi's Sarcoma-Associated Herpesvirus ORF31 with ORF34 and ORF24 Is Critical for Late Gene Expression. J Virol 89, 6148-6154, doi:10.1128/JVI.00272-15 (2015).
31 Davis, Z. H., Hesser, C. R., Park, J. & Glaunsinger, B. A. Interaction between ORF24 and ORF34 in the Kaposi's Sarcoma-Associated Herpesvirus Late Gene Transcription Factor Complex Is Essential for Viral Late Gene Expression. J Virol 90, 599-604, doi:10.1128/jvi.02157-15 (2016).
32 Gong, D. et al. Kaposi's sarcoma-associated herpesvirus ORF18 and ORF30 are essential for late gene expression during lytic replication. J Virol 88, 11369-11382, doi:10.1128/JVI.00793-14 (2014).
33 Gelgor, A. et al. Viral Bcl-2 Encoded by the Kaposi's Sarcoma-Associated Herpesvirus Is Vital for Virus Reactivation. J Virol 89, 5298-5307, doi:10.1128/jvi.00098-15 (2015).
34 Liang, Q. et al. Identification of the Essential Role of Viral Bcl-2 for Kaposi's Sarcoma-Associated Herpesvirus Lytic Replication. J Virol 89, 5308-5317, doi:10.1128/jvi.00102-15 (2015).
35 Kreitler, D. et al. The assembly domain of the small capsid protein of Kaposi's sarcoma-associated herpesvirus. J Virol 86, 11926-11930, doi:10.1128/JVI.01430-12 (2012).
36 Sathish, N. & Yuan, Y. Functional characterization of Kaposi's sarcoma-associated herpesvirus small capsid protein by bacterial artificial chromosome-based mutagenesis. Virology 407, 306-318, doi:10.1016/j.virol.2010.08.017 (2010).
37 Wu, J. J. et al. ORF33 and ORF38 of Kaposi's Sarcoma-Associated Herpesvirus Interact and Are Required for Optimal Production of Infectious Progeny Viruses. J Virol 90, 1741-1756, doi:10.1128/JVI.02738-15 (2016).
38 Brulois, K. F. et al. Construction and manipulation of a new Kaposi's sarcoma-associated herpesvirus bacterial artificial chromosome clone. J Virol 86, 9708-9720, doi:10.1128/JVI.01019-12 (2012).
39 DUnn-Kittenplon, D. D., Kalt, I., Lellouche, J. M. & Sarid, R. The KSHV portal protein ORF43 is essential for the production of infectious viral particles. Virology 529, 205-215, doi:10.1016/j.virol.2019.01.028 (2019).
40 Gong, D. et al. A Herpesvirus Protein Selectively Inhibits Cellular mRNA Nuclear Export. Cell Host Microbe 20, 642-653, doi:10.1016/j.chom.2016.10.004 (2016).
41 Kleer, M., MacNeil, G., Adam, N., Pringle, E. S. & Corcoran, J. A. A Panel of Kaposi's Sarcoma-Associated Herpesvirus Mutants in the Polycistronic Kaposin Locus for Precise Analysis of Individual Protein Products. J Virol 96, e0156021, doi:10.1128/JVI.01560-21 (2022).
42 Morgens, D. W., Nandakumar, D., Didychuk, A. L., Yang, K. J. & Glaunsinger, B. A. A Two-tiered functional screen identifies herpesviral transcriptional modifiers and their essential domains. PLoS Pathog 18, e1010236, doi:10.1371/journal.ppat.1010236 (2022).
43 Myoung, J. & Ganem, D. Generation of a doxycycline-inducible KSHV producer cell line of endothelial origin: maintenance of tight latency with efficient reactivation upon induction. J Virol Methods 174, 12-21, doi:10.1016/j.jviromet.2011.03.012 (2011).
44 Ballestas, M. E., Chatis, P. A. & Kaye, K. M. Efficient persistence of extrachromosomal KSHV DNA mediated by latency-associated nuclear antigen. Science 284, 641-644, doi:10.1126/science.284.5414.641 (1999).
45 Coscoy, L. & Ganem, D. A viral protein that selectively downregulates ICAM-1 and B7-2 and modulates T cell costimulation. J Clin Invest 107, 1599-1606, doi:10.1172/JCI12432 (2001).
46 Ishido, S., Wang, C., Lee, B. S., Cohen, G. B. & Jung, J. U. Downregulation of major histocompatibility complex class I molecules by Kaposi's sarcoma-associated herpesvirus K3 and K5 proteins. J Virol 74, 5300-5309, doi:10.1128/jvi.74.11.5300-5309.2000 (2000).
47 Lukac, D. M., Kirshner, J. R. & Ganem, D. Transcriptional activation by the product of open reading frame 50 of Kaposi's sarcoma-associated herpesvirus is required for lytic viral reactivation in B cells. J Virol 73, 9348-9361, doi:10.1128/jvi.73.11.9348-9361.1999 (1999).
48 Sun, R. et al. A viral gene that activates lytic cycle expression of Kaposi's sarcoma-associated herpesvirus. Proc Natl Acad Sci USA 95, 10866-10871, doi:10.1073/pnas.95.18.10866 (1998).
49 Endres, M. J., Garlisi, C. G., Xiao, H., Shan, L. & Hedrick, J. A. The Kaposi's sarcoma-related herpesvirus (KSHV)-encoded chemokine vMIP-I is a specific agonist for the CC chemokine receptor (CCR)8. J Exp Med 189, 1993-1998, doi:10.1084/jem.189.12.1993 (1999).
50 Nicholas, J. et al. Kaposi's sarcoma-associated human herpesvirus-8 encodes homologues of macrophage inflammatory protein-1 and interleukin-6. Nat Med 3, 287-292, doi:10.1038/nm0397-287 (1997).
51 Fuld, S., Cunningham, C., Klucher, K., Davison, A. J. & Blackbourn, D. J. Inhibition of interferon signaling by the Kaposi's sarcoma-associated herpesvirus full-length viral interferon regulatory factor 2 protein. J Virol 80, 3092-3097, doi:10.1128/JVI.80.6.3092-3097.2006 (2006).
52 Richard M. Longnecker, E. K., Jeffrey I. Cohen. Fields Virology 6th edn, Vol. 2 Chapter 61 (Lippincott and Williams, 2013).
53 Zhang, Z. et al. Genome-wide mutagenesis reveals that ORF7 is a novel VZV skin-tropic factor. PLoS Pathog 6, e1000971, doi:10.1371/journal.ppat.1000971 (2010).
54 Campbell, T. B. et al. Relationship of human herpesvirus 8 peripheral blood virus load and Kaposi's sarcoma clinical stage. AIDS 14, 2109-2116, doi:10.1097/00002030-200009290-00006 (2000).
55 Oksenhendler, E. et al. High levels of human herpesvirus 8 viral load, human interleukin-6, interleukin-10, and C reactive protein correlate with exacerbation of multicentric castleman disease in HIV-infected patients. Blood 96, 2069-2073 (2000).
56 Fardet, L. et al. Human herpesvirus 8-associated hemophagocytic lymphohistiocytosis in human immunodeficiency virus-infected patients. Clin Infect Dis 37, 285-291, doi:10.1086/375224 (2003).
57 Chen, J. et al. Activation of latent Kaposi's sarcoma-associated herpesvirus by demethylation of the promoter of the lytic transactivator. Proc Natl Acad Sci USA 98, 4119-4124, doi:10.1073/pnas.051004198 (2001).
58 Sozzani, S. et al. The viral chemokine macrophage inflammatory protein-II is a selective Th2 chemoattractant. Blood 92, 4036-4039 (1998).
59 Li, Q., Means, R., Lang, S. & Jung, J. U. Downregulation of gamma interferon receptor 1 by Kaposi's sarcoma-associated herpesvirus K3 and K5. J Virol 81, 2117-2127, doi:10.1128/JVI.01961-06 (2007).
60 Wang, H. W., Sharp, T. V., Koumi, A., Koentges, G. & Boshoff, C. Characterization of an anti-apoptotic glycoprotein encoded by Kaposi's sarcoma-associated herpesvirus which resembles a spliced variant of human survivin. EMBO J 21, 2602-2615, doi:10.1093/emboj/21.11.2602 (2002).
61 Liang, Q. et al. Kaposi's sarcoma-associated herpesvirus K7 modulates Rubicon-mediated inhibition of autophagosome maturation. J Virol 87, 12499-12503, doi:10.1128/JVI.01898-13 (2013).
62 Vieira, J. & O'Hearn, P. M. Use of the red fluorescent protein as a marker of Kaposi's sarcoma-associated herpesvirus lytic gene expression. Virology 325, 225-240, doi:10.1016/j.virol.2004.03.049 (2004).
63 de Jesus, O. et al. Updated Epstein-Barr virus (EBV) DNA sequence and analysis of a promoter for the BART (CST, BARF0) RNAs of EBV. J Gen Virol 84, 1443-1450, doi:10.1099/vir.0.19054-0 (2003).
64 McGeoch, D. J. et al. The complete DNA sequence of the long unique region in the genome of herpes simplex virus type 1. J Gen Virol 69 (Pt 7), 1531-1574, doi:10.1099/0022-1317-69-7-1531 (1988).
65 Murphy, E. et al. Coding potential of laboratory and clinical strains of human cytomegalovirus. Proc Natl Acad Sci USA 100, 14976-14981, doi:10.1073/pnas.2136652100 (2003).
66 Virgin, H. W. t. et al. Complete sequence and genomic analysis of murine gammaherpesvirus 68. J Virol 71, 5894-5904, doi:10.1128/JVI.71.8.5894-5904.1997 (1997).
67 AuCoin, D. P. et al. Amplification of the Kaposi's sarcoma-associated herpesvirus/human herpesvirus 8 lytic origin of DNA replication is dependent upon a cis-acting AT-rich region and an ORF50 response element and the trans-acting factors ORF50 (K-Rta) and K8 (K-bZIP). Virology 318, 542-555, doi:10.1016/j.virol.2003.10.016 (2004).
68 Davison, A. J. & Stow, N. D. New genes from old: redeployment of dUTPase by herpesviruses. J Virol 79, 12880-12892, doi:10.1128/JVI.79.20.12880-12892.2005 (2005).
69 Castaneda, A. F. & Glaunsinger, B. A. The Interaction between ORF18 and ORF30 Is Required for Late Gene Expression in Kaposi's Sarcoma-Associated Herpesvirus. J Virol 93, doi:10.1128/JVI.01488-18 (2019).
70 Gong, D. et al. DNA-Packing Portal and Capsid-Associated Tegument Complexes in the Tumor Herpesvirus KSHV. Cell 178, 1329-1343 e1312, doi:10.1016/j.cell.2019.07.035 (2019).
71 Dai, X., Gong, D., Wu, T. T., Sun, R. & Zhou, Z. H. Organization of capsid-associated tegument components in Kaposi's sarcoma-associated herpesvirus. J Virol 88, 12694-12702, doi:10.1128/JVI.01509-14 (2014).
72 Castaneda, A. F. et al. The gammaherpesviral TATA-box-binding protein directly interacts with the CTD of host RNA Pol II to direct late gene transcription. PLoS Pathog 16, e1008843, doi:10.1371/journal.ppat.1008843 (2020).
73 Dai, X. et al. Structure and mutagenesis reveal essential capsid protein interactions for KSHV replication. Nature 553, 521-525, doi:10.1038/nature25438 (2018).
74 Zhu, F. X., Chong, J. M., Wu, L. & Yuan, Y. Virion proteins of Kaposi's sarcoma-associated herpesvirus. J Virol 79, 800-811, doi:10.1128/JVI.79.2.800-811.2005 (2005).
75 Zhu, F. X. & Yuan, Y. The ORF45 protein of Kaposi's sarcoma-associated herpesvirus is associated with purified virions. J Virol 77, 4221-4230, doi:10.1128/jvi.77.7.4221-4230.2003 (2003).
76 Sathish, N., Zhu, F. X. & Yuan, Y. Kaposi's sarcoma-associated herpesvirus ORF45 interacts with kinesin-2 transporting viral capsid-tegument complexes along microtubules. PLoS Pathog 5, e1000332, doi:10.1371/journal.ppat.1000332 (2009).
77 Li, X. et al. ORF45-Mediated Prolonged c-Fos Accumulation Accelerates Viral Transcription during the Late Stage of Lytic Replication of Kaposi's Sarcoma-Associated Herpesvirus. J Virol 89, 6895-6906, doi:10.1128/JVI.00274-15 (2015).
78 Li, W. et al. Kaposi's Sarcoma-Associated Herpesvirus Inhibitor of cGAS (KicGAS), Encoded by ORF52, Is an Abundant Tegument Protein and Is Required for Production of Infectious Progeny Viruses. J Virol 90, 5329-5342, doi:10.1128/JVI.02675-15 (2016).
79 Gregory, S. M. et al. Discovery of a viral NLR homolog that inhibits the inflammasome. Science 331, 330-334, doi:10.1126/science.1199478 (2011).
80 Watanabe, T. et al. Kaposi's Sarcoma-Associated Herpesvirus ORF66 Is Essential for Late Gene Expression and Virus Production via Interaction with ORF34. J Virol 94, doi:10.1128/JVI.01300-19 (2020).
81 Luitweiler, E. M. et al. Interactions of the Kaposi's Sarcoma-associated herpesvirus nuclear egress complex: ORF69 is a potent factor for remodeling cellular membranes. J Virol 87, 3915-3929, doi:10.1128/JVI.03418-12 (2013).
82 Didychuk, A. L. et al. A pentameric protein ring with novel architecture is required for herpesviral packaging. Elife 10, doi:10.7554/eLife.62261 (2021).
83 Cheng, E. H. et al. A Bcl-2 homolog encoded by Kaposi sarcoma-associated virus, human herpesvirus 8, inhibits apoptosis but does not heterodimerize with Bax or Bak. Proc Natl Acad Sci USA 94, 690-694, doi:10.1073/pnas.94.2.690 (1997).
84 Gonzalez, C. M. et al. Identification and characterization of the Or49 protein of Kaposi's sarcoma-associated herpesvirus. J Virol 80, 3062-3070, doi:10.1128/JVI.80.6.3062-3070.2006 (2006).
85 Liu, D., Wang, Y. & Yuan, Y. Kaposi's Sarcoma-Associated Herpesvirus K8 Is an RNA Binding Protein That Regulates Viral DNA Replication in Coordination with a Noncoding RNA. J Virol 92, doi:10.1128/JVI.02177-17 (2018).
86 Rossetto, C., Yamboliev, I. & Pari, G. S. Kaposi's sarcoma-associated herpesvirus/human herpesvirus 8 K-bZIP modulates latency-associated nuclear protein-mediated suppression of lytic origin-dependent DNA synthesis. J Virol 83, 8492-8501, doi:10.1128/JVI.00922-09 (2009).
87 Nabiee, R., Syed, B., Ramirez Castano, J., Lalani, R. & Totonchy, J. E. An Update of the Virion Proteome of Kaposi Sarcoma-Associated Herpesvirus. Viruses 12, doi:10.3390/v12121382 (2020).
88 Cheng, A. Z. et al. A Conserved Mechanism of APOBEC3 Relocalization by Herpesviral Ribonucleotide Reductase Large Subunits. J Virol 93, doi:10.1128/JVI.01539-19 (2019).
89 Mullick, J., Bernet, J., Singh, A. K., Lambris, J. D. & Sahu, A. Kaposi's sarcoma-associated herpesvirus (human herpesvirus 8) open reading frame 4 protein (kaposica) is a functional homolog of complement control proteins. J Virol 77, 3878-3881, doi:10.1128/jvi.77.6.3878-3881.2003 (2003).
90 Park, J., Lee, D., Seo, T., Chung, J. & Choe, J. Kaposi's sarcoma-associated herpesvirus (human herpesvirus-8) open reading frame 36 protein is a serine protein kinase. J Gen Virol 81, 1067-1071, doi:10.1099/0022-1317-81-4-1067 (2000).
91 Lee, H. et al. Identification of an immunoreceptor tyrosine-based activation motif of KI transforming protein of Kaposi's sarcoma-associated herpesvirus. Mol Cell Biol 18, 5219-5228, doi:10.1128/MCB.18.9.5219 (1998).
92 Wang, L., Dittmer, D. P., Tomlinson, C. C., Fakhari, F. D. & Damania, B. Immortalization of primary endothelial cells by the KI protein of Kaposi's sarcoma-associated herpesvirus. Cancer Res 66, 3658-3666, doi:10.1158/0008-5472.CAN-05-3680 (2006).
93 Li, H., Wang, H. & Nicholas, J. Detection of direct binding of human herpesvirus 8-encoded interleukin-6 (vIL-6) to both gp130 and IL-6 receptor (IL-6R) and identification of amino acid residues of vIL-6 important for IL-6R-dependent and independent signaling. J Virol 75, 3325-3334, doi:10.1128/JVI.75.7.3325-3334.2001 (2001).
94 Aoki, Y. et al. Angiogenesis and hematopoiesis induced by Kaposi's sarcoma-associated herpesvirus-encoded interleukin-6. Blood 93, 4034-4043 (1999).
95 Kledal, T. N. et al. A broad-spectrum chemokine antagonist encoded by Kaposi's sarcoma-associated herpesvirus. Science 277, 1656-1659, doi:10.1126/science.277.5332.1656 (1997).
96 Stine, J. T. et al. KSHV-encoded CC chemokine vMIP-III is a CCR4 agonist, stimulates angiogenesis, and selectively chemoattracts TH2 cells. Blood 95, 1151-1157 (2000).
97 Nascimento, R., Dias, J. D. & Parkhouse, R. M. The conserved UL24 family of human alpha, beta and gamma herpesviruses induces cell cycle arrest and inactivation of the cyclinB/cdc2 complex. Arch Virol 154, 1143-1149, doi:10.1007/s00705-009-0420-y (2009).
98 Burysek, L. et al. Functional analysis of human herpesvirus 8-encoded viral interferon regulatory factor 1 and its association with cellular interferon regulatory factors and p300. J Virol 73, 7334-7342, doi:10.1128/JVI.73.9.7334-7342.1999 (1999).
99 Choi, Y. B. & Nicholas, J. Bim nuclear translocation and inactivation by viral interferon regulatory factor. PLoS Pathog 6, e1001031, doi:10.1371/journal.ppat.1001031 (2010).
100 Lee, H. R. et al. Kaposi's sarcoma-associated herpesvirus viral interferon regulatory factor 4 targets MDM2 to deregulate the p53 tumor suppressor pathway. J Virol 83, 6739-6747, doi:10.1128/JVI.02353-08 (2009).
101 Marcos-Villar, L. et al. Kaposi's sarcoma-associated herpesvirus protein LANA2 disrupts PML oncogenic domains and inhibits PML-mediated transcriptional repression of the survivin gene. J Virol 83, 8849-8858, doi:10.1128/JVI.00339-09 (2009).
102 Joo, C. H. et al. Inhibition of interferon regulatory factor 7 (IRF7)-mediated interferon signal transduction by the Kaposi's sarcoma-associated herpesvirus viral IRF homolog vIRF3. J Virol 81, 8282-8292, doi:10.1128/JVI.00235-07 (2007).
103 Burysek, L. & Pitha, P. M. Latently expressed human herpesvirus 8-encoded interferon regulatory factor 2 inhibits double-stranded RNA-activated protein kinase. J Virol 75, 2345-2352, doi:10.1128/JVI.75.5.2345-2352.2001 (2001).
104 Sadler, R. et al. A complex translational program generates multiple novel proteins from the latently expressed kaposin (K12) locus of Kaposi's sarcoma-associated herpesvirus. J Virol 73, 5722-5730, doi:10.1128/JVI.73.7.5722-5730.1999 (1999).
105 Kliche, S. et al. Signaling by human herpesvirus 8 kaposin A through direct membrane recruitment of cytohesin-1. Mol Cell 7, 833-843, doi:10.1016/s1097-2765(01)00227-1 (2001).
106 Ballon, G., Chen, K., Perez, R., Tam, W. & Cesarman, E. Kaposi sarcoma herpesvirus (KSHV) vFLIP oncoprotein induces B cell transdifferentiation and tumorigenesis in mice. J Clin Invest 121, 1141-1153, doi:10.1172/JCI44417 (2011).
107 Sun, Q., Zachariah, S. & Chaudhary, P. M. The human herpes virus 8-encoded viral FLICE-inhibitory protein induces cellular transformation via NF-kappaB activation. J Biol Chem 278, 52437-52445, doi:10.1074/jbc.M304199200 (2003).
108 Punj, V. et al. Kaposi's sarcoma-associated herpesvirus-encoded viral FLICE inhibitory protein (vFLIP) K13 suppresses CXCR4 expression by upregulating miR-146a. Oncogene 29, 1835-1844, doi:10.1038/onc.2009.460 (2010).
109 Ojala, P. M. et al. The apoptotic v-cyclin-CDK6 complex phosphorylates and inactivates Bcl-2. Nat Cell Biol 2, 819-825, doi:10.1038/35041064 (2000).
110 Swanton, C. et al. Herpes viral cyclin/Cdk6 complexes evade inhibition by CDK inhibitor proteins. Nature 390, 184-187, doi:10.1038/36606 (1997).
111 Salata, C. et al. vOX2 glycoprotein of human herpesvirus 8 modulates human primary macrophages activity. J Cell Physiol 219, 698-706, doi:10.1002/jcp.21722 (2009).
112 Bais, C. et al. G-protein-coupled receptor of Kaposi's sarcoma-associated herpesvirus is a viral oncogene and angiogenesis activator. Nature 391, 86-89, doi:10.1038/34193 (1998).
113 Choi, J. K., Lee, B. S., Shim, S. N., Li, M. & Jung, J. U. Identification of the novel K15 gene at the rightmost end of the Kaposi's sarcoma-associated herpesvirus genome. J Virol 74, 436-446 (2000).
114 Glenn, M., Rainbow, L., Aurade, F., Davison, A. & Schulz, T. F. Identification of a spliced gene from Kaposi's sarcoma-associated herpesvirus encoding a protein with similarities to latent membrane proteins 1 and 2A of Epstein-Barr virus. J Virol 73, 6953-6963, doi:10.1128/JVI.73.8.6953-6963.1999 (1999).
115 Mortazavi, Y. et al. The Kaposi's Sarcoma-Associated Herpesvirus (KSHV) gH/gL Complex Is the Predominant Neutralizing Antigenic Determinant in KSHV-Infected Individuals. Viruses 12, doi:10.3390/v12030256 (2020).
116 Bernard Roizman, D. M. K., Richard J. Whitley. Fields Virology 6th edn, Vol. 2 Chapter 60 (Lippincott and Williams, 2013).
117 Ann M. Arvin, D. G. Feilds Virology 6th edn, Vol. 2 Chapter 63 (Lippincott and Williams, 2013).
118 Edward S. Mocarski, J., Thomas Shenk, Paul D. Griffiths, Robert F. Pass. Fields Virology 6th edn, Vol. 2 Chapter 62 (Lippincott and Williams, 2013).
119 Fixman, E. D., Hayward, G. S. & Hayward, S. D. trans-acting requirements for replication of Epstein-Barr virus ori-Lyt. J Virol 66, 5030-5039, doi:10.1128/JVI.66.8.5030-5039.1992 (1992).
120 Chiu, S. H. et al. Epstein-Barr virus BALF3 has nuclease activity and mediates mature virion production during the lytic cycle. J Virol 88, 4962-4975, doi:10.1128/JVI.00063-14 (2014).
121 Herrold, R. E., Marchini, A., Fruehling, S. & Longnecker, R. Glycoprotein 110, the Epstein-Barr virus homolog of herpes simplex virus glycoprotein B, is essential for Epstein-Barr virus replication in vivo. J Virol 70, 2049-2054, doi:10.1128/JVI.70.3.2049-2054.1996 (1996).
122 Marchini, A., Tomkinson, B., Cohen, J. I. & Kieff, E. BHRF1, the Epstein-Barr virus gene with homology to Bcl2, is dispensable for B-lymphocyte transformation and virus replication. J Virol 65, 5991-6000, doi:10.1128/JVI.65.11.5991-6000.1991 (1991).
123 Larrat, S. et al. Inhibition of Epstein-Barr virus replication by small interfering RNA targeting the Epstein-Barr virus protease gene. Antivir Ther 14, 655-662 (2009).
124 Henson, B. W., Perkins, E. M., Cothran, J. E. & Desai, P. Self-assembly of Epstein-Barr virus capsids. J Virol 83, 3877-3890, doi:10.1128/JVI.01733-08 (2009).
125 Oda, T., Imai, S., Chiba, S. & Takada, K. Epstein-Barr virus lacking glycoprotein gp85 cannot infect B cells and epithelial cells. Virology 276, 52-58, doi:10.1006/viro.2000.0531 (2000).
126 Gruffat, H., Kadjouf, F., Mariame, B. & Manet, E. The Epstein-Barr virus BcRF1 gene product is a TBP-like protein with an essential role in late gene expression. J Virol 86, 6023-6032, doi:10.1128/JVI.00159-12 (2012).
127 Jochum, S., Moosmann, A., Lang, S., Hammerschmidt, W. & Zeidler, R. The EBV immunoevasins vIL-10 and BNLF2a protect newly infected B cells from immune recognition and elimination. PLoS Pathog 8, e1002704, doi:10.1371/journal.ppat.1002704 (2012).
128 Borza, C. M. & Hutt-Fletcher, L. M. Epstein-Barr virus recombinant lacking expression of glycoprotein gp150 infects B cells normally but is enhanced for infection of epithelial cells. J Virol 72, 7577-7582, doi:10.1128/JVI.72.9.7577-7582.1998 (1998).
129 Pavlova, S. et al. An Epstein-Barr virus mutant produces immunogenic defective particles devoid of viral DNA. J Virol 87, 2011-2022, doi:10.1128/JVI.02533-12 (2013).
130 Watanabe, T. et al. The Epstein-Barr Virus BDLF4 Gene Is Required for Efficient Expression of Viral Late Lytic Genes. J Virol 89, 10120-10124, doi:10.1128/JVI.01604-15 (2015).
131 Hung, C. H. et al. Interaction Between BGLF2 and BBLF1 Is Required for the Efficient Production of Infectious Epstein-Barr Virus Particles. Front Microbiol 10, 3021, doi:10.3389/fmicb.2019.03021 (2019).
132 Watanabe, T. et al. Roles of Epstein-Barr virus BGLF3.5 gene and two upstream open reading frames in lytic viral replication in HEK293 cells. Virology 483, 44-53, doi:10.1016/j.virol.2015.04.007 (2015).
133 Murata, T. et al. Efficient production of infectious viruses requires enzymatic activity of Epstein-Barr virus protein kinase. Virology 389, 75-81, doi:10.1016/j.virol.2009.04.007 (2009).
134 Feederle, R., Bannert, H., Lips, H., Muller-Lantzsch, N. & Delecluse, H. J. The Epstein-Barr virus alkaline exonuclease BGLF5 serves pleiotropic functions in virus replication. J Virol 83, 4952-4962, doi:10.1128/JVI.00170-09 (2009).
135 Malik, A. K., Martinez, R., Muncy, L., Carmichael, E. P. & Weller, S. K. Genetic analysis of the herpes simplex virus type 1 UL9 gene: isolation of a LacZ insertion mutant and expression in eukaryotic cells. Virology 190, 702-715, doi:10.1016/0042-6822(92)90908-8 (1992).
136 Masud, H. et al. Epstein-Barr Virus BBRF2 Is Required for Maximum Infectivity. Microorganisms 7, doi:10.3390/microorganisms7120705 (2019).
137 Pavlova, S. et al. An Epstein-Barr virus mutant produces immunogenic defective particles devoid of viral DNA. Journal of virology 87, 2011-2022, doi:10.1128/jvi.02533-12 (2013).
138 Baines, J. D. & Roizman, B. The open reading frames UL3, UL4, UL10, and UL16 are dispensable for the replication of herpes simplex virus 1 in cell culture. J Virol 65, 938-944, doi:10.1128/JVI.65.2.938-944.1991 (1991).
139 Masud, H. et al. Epstein-Barr Virus BKRF4 Gene Product Is Required for Efficient Progeny Production. J Virol 91, doi:10.1128/JVI.00975-17 (2017).
140 Su, M. T. et al. Uracil DNA glycosylase BKRF3 contributes to Epstein-Barr virus DNA replication through physical interactions with proteins in viral DNA replication complex. J Virol 88, 8883-8899, doi:10.1128/jvi.00950-14 (2014).
141 Fixman, E. D., Hayward, G. S. & Hayward, S. D. Replication of Epstein-Barr virus oriLyt: lack of a dedicated virally encoded origin-binding protein and dependence on Zta in cotransfection assays. J Virol 69, 2998-3006, doi:10.1128/JVI.69.5.2998-3006.1995 (1995).
142 Watanabe, T. et al. The Epstein-Barr virus BRRF2 gene product is involved in viral progeny production. Virology 484, 33-40, doi:10.1016/j.virol.2015.05.010 (2015).
143 Yoshida, M. et al. The Epstein-Barr Virus BRRF1 Gene Is Dispensable for Viral Replication in HEK293 cells and Transformation. Sci Rep 7, 6044, doi:10.1038/s41598-017-06413-7 (2017).
144 Feederle, R. et al. The Epstein-Barr virus lytic program is controlled by the co-operative functions of two transactivators. EMBO J 19, 3080-3089, doi:10.1093/emboj/19.12.3080 (2000).
145 Lake, C. M. & Hutt-Fletcher, L. M. Epstein-Barr virus that lacks glycoprotein gN is impaired in assembly and infection. J Virol 74, 11162-11172, doi:10.1128/jvi.74.23.11162-11172.2000 (2000).
146 Yanagi, Y. et al. Initial Characterization of the Epstein(−)Barr Virus BSRF1 Gene Product. Viruses 11, doi:10.3390/v11030285 (2019).
147 Gruffat, H. et al. Epstein-Barr virus mRNA export factor EB2 is essential for production of infectious virus. J Virol 76, 9635-9644, doi:10.1128/jvi.76.19.9635-9644.2002 (2002).
148 Cohen, J. I. & Lekstrom, K. Epstein-Barr virus BARF1 protein is dispensable for B-cell transformation and inhibits alpha interferon secretion from mononuclear cells. J Virol 73, 7627-7632, doi:10.1128/JVI.73.9.7627-7632.1999 (1999).
149 Cheng, A. Z. et al. Epstein-Barr virus BORF2 inhibits cellular APOBEC3B to preserve viral genome integrity. Nat Microbiol 4, 78-88, doi:10.1038/s41564-018-0284-6 (2019).
150 Masud, H. et al. The BOLF1 gene is necessary for effective Epstein-Barr viral infectivity. Virology 531, 114-125, doi:10.1016/j.virol.2019.02.015 (2019).
151 Whitehurst, C. B. et al. Knockout of Epstein-Barr virus BPLF1 retards B-cell transformation and lymphoma formation in humanized mice. mBio 6, e01574-01515, doi:10.1128/mBio.01574-15 (2015).
152 Aubry, V. et al. Epstein-Barr virus late gene transcription depends on the assembly of a virus-specific preinitiation complex. J Virol 88, 12825-12838, doi:10.1128/JVI.02139-14 (2014).
153 Farina, A. et al. BFRFI of Epstein-Barr virus is essential for efficient primary viral envelopment and egress. J Virol 79, 3703-3712, doi:10.1128/JVI.79.6.3703-3712.2005 (2005).
154 Granato, M. et al. Deletion of Epstein-Barr virus BFLF2 leads to impaired viral DNA packaging and primary egress as well as to the production of defective viral particles. J Virol 82, 4042-4051, doi:10.1128/JVI.02436-07 (2008).

1. A Kaposi sarcoma associated herpesvirus (KSHV) mutant with inactivation or deletion of one or more of the open reading frames (ORFs) disclosed in Table 1.

2. A method of using the mutant viruses of claim 1 for analyzing the molecular, cellular, and immunological response to mutant virus infections.

3. A method of using the mutant viruses of claim 1 as a “helper-virus” in the production of other viral vectors, and/or the generation of live-attenuated vaccines.

4. A pair of primers for construction of a KSHV mutant of claim 1.

5. A pair of primers for construction of a KSHV mutant of claim 1, as disclosed in Table S1.

6. A method of using the primers of claim 4 for construction of a KSHV mutant.

7. A method of using the primers of claim 4 for mutagenesis having high fidelity (e.g. insert or remove a desired sequence with single nucleotide resolution), superior to other mutagenesis approaches like CRISPR.

8. A method for reconstituting mutant viruses using the primers of claim 4, using transfection, induction and/or tittering, the methods comprising a tractable workflow.

9. An artificial gene, such as protein expression plasmids, or gene product thereof, the gene comprising one of the KSHV essential or nonessential genes disclosed in Table 1, particularly the 27 new identified essential genes, as disclosed.

10. Use of a gene or gene product of claim 9 as an antiviral target.

11. Use of the artificial constructs of claim 9 in a high throughput, in-vitro drug screening assay to identify novel antivirals for KSHV, and other human herpesviruses.

12. Use of a gene or gene product of claim 9 in the production of a monoclonal antibody or nucleic acid therapy for KSHV infection.

13. A method for using a mutant according to claim 1 for construction of a gene-inactivation or rescued mutant or for tagging or introducing foreign genes into the KSHV genome, particularly for use in vector and vaccine development.

14. Use of growth properties of a viral mutant according to claim 1, with inactivation of non-essential genes as disclosed.

15. A method of using a non-essential gene of claim 9 in a live-attenuated vaccine to impart attenuated growth.

16. Use of a non-essential genes of claim 15 to produce live-attenuated vaccine candidates.

17. A method for screening KSHV mutants of claim 1 in human cell lines as disclosed.

18. An opportunistic factor that functions to suppress KSHV spontaneous reactivation, for use in the treatment of KSHV infection, as disclosed.

19. Use of the opportunistic factor of claim 18, as both the modulators of immune environment/response and regulators of viral reactivation/replication, as disclosed, or use of the opportunistic factor of claim 18 for KSHV therapy; for example, over-expressing an opportunistic factor that functions to suppress KSHV spontaneous reactivation, find use in the treatment of KSHV infection.

20. A method of expressing an opportunistic factor of claim 18, that functions to suppress KSHV spontaneous reactivation, for use in the treatment of KSHV infection.
